(19) United States
(12) Patent Application Publication
(10) Pub. No.: US 2011/0223630 A1
(43) Pub. Date: Sep. 15, 2011

(54) METHOD FOR SCREENING FOR COMPOUNDS THAT INHIBIT NEURODEGENERATION

(76) Inventors: Anatoly Nikolaev, Burlingame, CA (US); Marc Tessier-Lavigne, Atherton, CA (US)

(21) Appl. No.: 12/997,297

(22) PCT Filed: Jun. 12, 2009
(86) PCT No.: PCT/US2009/047255

§ 371 Date: May 3, 2011

Related U.S. Application Data
(60) Provisional application No. 61/061,062, filed on Jun. 12, 2008.

(51) Int. Cl. C12Q 1/02

(52) U.S. Cl. 435/29

(57) ABSTRACT

Methods for screening for compounds that inhibit neurodegeneration are presented. Shedding of APP can be a useful marker for neurodegeneration and compounds that inhibit shedding of APP are useful as inhibitors of neurodegeneration. Such compounds may be useful in treatment and/or prevention of various neurological diseases, disorders and neuronal damage and may enhance growth, regeneration or survival of mammalian neuronal cells or tissue.

### CROSS-REFERENCE TO RELATED APPLICATIONS

This application is a Continuation-in-Part of PCT Application No. PCT/US2007/88521, filed Dec. 21, 2007, which claims benefit of U.S. Provisional Application Nos. 60/871,528, filed Dec. 22, 2006 and 60/900,848, filed Feb. 12, 2007. This application claims benefit of U.S. Provisional Application No. 61/061,062, filed Jun. 12, 2008. The contents of these applications are hereby incorporated by reference in their entirety.

### FIELD OF THE INVENTION

The present invention relates generally to methods of screening for compounds that inhibit degeneration of neurons. More specifically, the methods involve screening for compounds that inhibit shedding of APP from neurons upon a triggering event for neuronal degeneration.

### BACKGROUND OF THE INVENTION

Various ligands and receptors belonging to the tumor necrosis factor (TNF) superfamily have been identified in the art. Included among such ligands are tumor necrosis factor-alpha (“TNF-alpha”), tumor necrosis factor-beta (“TNF-beta” or “lymphotoxin-alpha”), lymphotoxin-beta (“LT-beta”), CD30 ligand, CD27 ligand, CD40 ligand, OX-40 ligand, 4-1BB ligand, LIGHT, Apo-1 ligand (also referred to as Fas ligand or CD95 ligand), Apo-2 ligand (also referred to as Apo2L or TRAIL), Apo-3 ligand (also referred to as TWEAK), APRIL, OPG ligand (also referred to as RANK ligand, ODF, or TRANCE), and TALL-1 (also referred to as BlyS, BAFF or THANK) (See, e.g., Ashkenazi, Nature Review, 2:420-430 (2002); Ashkenazi and Dixit, Science, 281:1305-1308 (1998); Ashkenazi and Dixit, Curr. Opin. Cell Biol., 11:255-260 (2000); Golstein, Curr. Biol., 7:750-753 (1997) Wallach, CYTOKINE_REFERENCE, Academic Press, 2000, pages 377-411; Locksley et al., Cell, 104:487-501 (2001); Gruss and Dower, Blood, 85:3378-3404 (1995); Schmid et al., Proc. Natl. Acad. Sci. USA, 83:1881 (1986); Dealtry et al., Eur. J. Immunol., 17:689 (1987); Pitti et al., J. Biol. Chem., 271:12687-12690 (1996); Wiley et al., Immunity, 3:673-682 (1995); Browning et al., Cell, 72:847-856 (1993); Armitage et al. Nature, 357:80-82 (1992), WO 97/01633 published Jan. 16, 1997; WO 97/25428 published Jul. 17, 1997; Marsters et al., Curr. Biol., 8:525-528 (1998); Chicheportiche et al., J. Biol. Chem., 272:32401-32410 (1997); Hahne et al., J. Exp. Med., 188:1185-1190 (1998); WO98/28426 published Jul. 2, 1998; WO98/46751 published Oct. 22, 1998; WO/98/18921 published May 7, 1998; Moore et al., Science, 285:260-263 (1999); Shu et al., J. Leukocyte Biol., 65:680 (1999); Schneider et al., J. Exp. Med., 189:1747-1756 (1999); Mukhopadhyay et al., J. Biol. Chem., 274:15978-15981 (1999)).

Induction of various cellular responses mediated by such TNF family ligands is typically initiated by their binding to specific cell receptors. Included among the members of the TNF receptor superfamily identified to date are TNFR1, TNFR2, p75-NGFR, TACI, GITR, CD27, OX-40, CD30, CD40, HVEM, Fas (also referred to as Apo-1 or CD95), DR4 (also referred to as TRAIL-R1), DR5 (also referred to as Apo-2 or TRAIL-R2), DR6 (also referred to as TR9, also known in literature as TNF Receptor Superfamily Member 21 or TNFRSF21), DcR1, DcR2, osteoprotegerin (OPG), RANK and Apo-3 (also referred to as DR3 or TRAMP) (see, e.g., Ashkenazi, Nature Reviews, 2:420-430 (2002); Ashkenazi and Dixit, Science, 281:1305-1308 (1998); Ashkenazi and Dixit, Curr. Opin. Cell Biol., 11:255-260 (2000); Golstein, Curr. Biol., 7:750-753 (1997) Wallach, Cytokine Reference, Academic Press, 2000, pages 377-411; Locksley et al., Cell, 104:487-501 (2001); Gruss and Dower, Blood, 85:3378-3404 (1995); Hohman et al., J. Biol. Chem., 264:14927-14934 (1989); Brockhaus et al., Proc. Natl. Acad. Sci. USA, 87:3127-3131 (1990); EP 417,563, published Mar. 20, 1991; Loetscher et al., Cell, 61:351 (1990); Schall et al., Cell, 61:361 (1990); Smith et al., Science, 248:1019-1023 (1990); Lewis et al., Proc. Natl. Acad. Sci. USA, 88:2830-2834 (1991); Goodwin et al., Mol. Cell. Biol., 11:3020-3026 (1991); Stamenkovic et al., EMBO J., 8:1403-1410 (1989); Mallett et al., EMBO J., 9:1063-1068 (1990); Anderson et al., Nature, 390:175-179 (1997); Chicheportiche et al., J. Biol. Chem., 272:32401-32410 (1997); Pan et al., Science, 276:111-113 (1997); Pan et al., Science, 277:815-818 (1997); Sheridan et al., Science, 277:818-821 (1997); Degli-Esposti et al., J. Exp. Med., 186:1165-1170 (1997); Marsters et al., Curr. Biol., 7:1003-1006 (1997); Tsuda et al., BBRC, 234:137-142 (1997); Nocentini et al., Proc. Natl. Acad. Sci. USA, 94:6216-6221 (1997); vonBulow et al., Science, 278:138-141 (1997); Johnson et al., Cell, 47:545-554 (1986); Radeke et al., Nature, 325:593-597 (1987); Pan et al., FEBS Lett., 431:351-356 (1998)).

Most of these TNF receptor family members share the typical structure of cell surface receptors including extracellular, transmembrane and intracellular regions, while others are found naturally as soluble proteins lacking a transmembrane and intracellular domain. The extracellular portion of typical TNFRs contains a repetitive amino acid sequence pattern of multiple cysteine-rich domains (CRDs), starting from the NH₂-terminus.

For reviews of the TNF family of ligands and receptors generally, see, e.g., Wallach, CYTOKINE REFERENCE, Academic Press, 2000, pages 377-411; Locksley et al., Cell, 104:487-501 (2001); Ware, Cytokine & Growth Factor Reviews, 14:181-184 (2003); Liu et al., Immunity, 15(1):23-34 (2001) and Bossen et al., J. Biol. Chem. 281(20):13964-71 (2006).

The TNFR family member called DR6 receptor (also referred to in literature as “TR9”; also known in literature as TNF Receptor Superfamily Member 21 or TNFRSF21) has been described as a type I transmembrane receptor having four extracellular cysteine-rich motifs and a cytoplasmic death domain structure (Pan et al., FEBS Lett., 431:351-356 (1998); see also U.S. Pat. Nos. 6,358,508; 6,667,390; 6,919,078; 6,949,358). It has been reported that overexpression of DR6 in certain transfected cell lines resulted in apoptosis and activation of both NF-kB and JNK (Pan et al., FEBS Letters, 431:351-356 (1998)). In a DR6-deficient mouse model, T cells were substantially impaired in JNK activation, and when DR6(−/−) mice were challenged with protein antigen, their T cells were found to hyperproliferate and display a profound polarization toward a Th2 response (whereas Th1 differentiation was not equivalently affected) (Zhao et al., J. Exp. Med., 194:1441-1448 (2001)). It was further reported that targeted disruption of DR6 resulted in enhanced T helper 2 (Th2) differentiation in vitro (Zhao et al., supra). Various uses of DR6 agonists or antagonists in modulating B-cell mediated conditions were described in US 2005/0069540 published Mar. 31, 2005. The DR6 receptor may play a role in regulating airway inflammation in the OVA-induced mouse model of asthma (Venkataraman et al., Immunol. Lett., 106:42-47 (2006)).

Using a myelin oligodendrocyte glycoprotein (MOG(35-55))-induced model of experimental autoimmune encephalomyelitis, DR6−/− mice were found to be highly resistant to both the onset and the progression of CNS disease compared with wild-type (WT) littermates. Thus, DR6 may be involved in regulating leukocyte infiltration and function in the induction and progression of experimental autoimmune encephalomyelitis (Schmidt et al., J. Immunol., 175:2286-2292 (2005)).

While various TNF ligand and receptor family members have been identified as having diverse biological activities and properties, few such ligands and receptors have been reported to be involved in neurological-related functions. For example, WO2004/071528 published Aug. 26, 2004 describes inhibition of the CD95 (Fas) ligand/receptor complex in a murine model to treat spinal cord injury.

### SUMMARY OF THE INVENTION

In embodiments of the invention, there are provided isolated death receptor 6 (“DR6”) antagonists. Certain embodiments of the antagonists disclosed herein inhibit or block interaction between DR6 and one or more of its cognate ligand(s). In preferred embodiments, the DR6 antagonists disclosed herein inhibit or block interaction between DR6 and its cognate ligand, amyloid precursor protein (“APP”). Embodiments of DR6 antagonists may comprise antibodies, such as DR6 or APP antibodies. Such DR6 antagonistic antibodies may, for example, be monoclonal antibodies, chimeric antibodies, humanized antibodies, or human antibodies. In certain embodiments of the invention, the DR6 antagonist may comprise an anti-DR6 antibody which binds DR6 extracellular domain polypeptide or fragment thereof, and optionally may bind a DR6 polypeptide comprising amino acids 1-349 or 42-349 of FIG. 1A. Alternatively, the DR6 antagonist may comprise an anti-APP antibody which binds an APP polypeptide, and optionally may bind an APP polypeptide comprising amino acids 66-81 of FIG. 1B (SEQ ID NO: 6).

DR6 antagonists contemplated also include DR6 immunoadhesins, DR6 variants, DR6 fragments, covalently modified forms thereof, or fusion proteins thereof, as well as small molecule antagonists. By way of example, DR6 antagonists may include pegylated DR6 or soluble extracellular domain forms of DR6 fused to heterologous sequences such as epitope tags, antibody fragments, such as human Fc, or leucine zippers.

Illustrative embodiments of the invention also include methods of inhibiting or blocking binding of DR6 to APP comprising exposing DR6 polypeptide and/or APP polypeptide to one or more DR6 antagonists under conditions wherein binding of DR6 to APP is inhibited. Typical DR6 antagonists used in such methods include antibodies that bind DR6 or APP, as well as soluble DR6 polypeptides. Optionally, DR6 antagonists are selected for use in these methods by observing their ability to inhibit binding between DR6 and APP. In certain embodiments of the invention, such methods are used for example to inhibit apoptosis and/or to enhance the growth and/or survival of neuronal cells in an in vitro tissue culture. The methods contemplate the use of a single type of DR6 antagonist molecule or a combination of two or more types of DR6 antagonists.

Embodiments of the invention also provide methods for enhancing growth or regeneration or survival of neuronal cells or tissue in mammals, comprising administering to a mammal an effective amount of DR6 antagonist. In optional embodiments, administration of DR6 antagonist enhances growth and blocks cell death and degeneration of neuronal cells or tissue in said mammal. The neuronal cells or tissue may comprise, for example, motor neurons, sensory neurons, commissural neurons, axons, microglia, and/or oligodendrocytes. In some embodiments of the invention, the DR6 antagonist used in such methods may comprise an antibody that binds APP and inhibits its ability to bind DR6. In other embodiments of the invention, the DR6 antagonist used in such methods may comprise an antibody that binds DR6 and inhibits its ability to bind APP. Alternatively, the DR6 antagonist may comprise a DR6 immunoadhesin, DR6 polypeptide linked to a nonproteinaceous polymer selected from the group consisting of polyethylene glycol, polypropylene glycol, and polyoxyalkylene, or a DR6 polypeptide variant. The DR6 immunoadhesins employed in the methods may comprise a soluble DR6 receptor fused to a Fc region of an immunoglobulin. Still further, DR6 antagonists of the invention may include small molecules.

Embodiments of the invention also provide methods for treating neurological disorders comprising administering to a mammal an effective amount of DR6 antagonist. In optional embodiments, the methods comprise treating Alzheimer's disease in a mammal. The DR6 antagonist used in such methods may comprise an antibody that binds APP and inhibits its ability to bind DR6. The DR6 antagonist may also comprise a DR6 antibody. Alternatively, the DR6 antagonist may comprise a DR6 immunoadhesin, DR6 polypeptide linked to a nonproteinaceous polymer selected from the group consisting of polyethylene glycol, polypropylene glycol, and polyoxyalkylene, DR6 antibody or a DR6 variant. The DR6 immunoadhesins employed in the methods may comprise a soluble DR6 receptor fused to a Fc region of an immunoglobulin. The anti-DR6 antibodies employed in the methods may bind a DR6 receptor comprising amino acids 1-349 or 42-349 of FIG. 1A.

Embodiments of the invention also include methods for diagnosing a patient with a neurological disorder or susceptible to a neurological disorder, comprising obtaining a sample from the patient and testing the sample for the presence of a DR6 polypeptide variant having a polypeptide sequence that differs from the DR6 polypeptide sequence of SEQ ID NO: 1. Typically in such methods the polypeptide variant is identified as having an affinity for an APP polypeptide that differs from the affinity observed for the DR6 polypeptide sequence of SEQ ID NO: 1.

Embodiments of the invention also provide methods for identifying a molecule of interest which inhibits binding of DR6 to APP. Such methods may comprise combining DR6 and APP in the presence or absence of a molecule of interest; and then detecting inhibition of binding of DR6 to APP in the presence of said molecule of interest. Optionally such methods are performed using mammalian cells expressing DR6 on the cell surface; and further include detecting inhibition of DR6 activation or signaling. Embodiments of the invention further include molecules identified by such methods. Optionally, the molecule of interest is antibody that binds APP, an antibody that binds DR6 or a soluble DR6 polypeptide.

Embodiments of the invention also provide antibodies which are capable of specifically binding to APP ligand, DR6 receptor and/or are capable of modulating biological activities associated with DR6 and/or its ligand(s) and/or co-receptors, and are useful in the treatment of various neurological disorders. In particular embodiments, there are provided antibodies which specifically bind to an extracellular domain sequence of DR6 polypeptide (described further in the Examples below). Typical antibodies are those which bind APP or DR6 and which are further selected for their ability to inhibit binding between DR6 and APP. Optionally, the antibody is a monoclonal antibody. Optionally, the monoclonal antibody comprises the 3F4.4.8, 4B6.9.7, or 1E5.5.7 antibody secreted by the hybridoma deposited with ATCC as accession number PTA-8095, PTA-8094, or PTA-8096, respectively.

Also provided are antibodies which bind to the same epitope as the epitope to which the 3F4.4.8, 4B6.9.7, or 1E5.5.7 monoclonal antibody produced by the hybridoma cell line deposited as ATCC accession number PTA-8095, PTA-8094, or PTA-8096, respectively, binds. In one aspect, the invention concerns an anti-DR6 antibody comprising 3F4.4.8, 4B6.9.7, or 1E5.5.7 antibody shows at least the same affinity for DR6, and/or exhibits at least the same biological activity and/or potency as antibody 3F4.4.8, 4B6.9.7, or 1E5.5.7.

In yet other particular embodiments, there is provided the hybridoma cell line which produces monoclonal antibody 3F4.4.8, 4B6.9.7, or 1E5.5.7 and deposited with ATCC as accession number PTA-8095, PTA-8094, or PTA-8096, respectively, and the monoclonal antibody 3F4.4.8, 4B6.9.7, or 1E5.5.7 secreted by the hybridoma deposited with ATCC as accession number PTA-8095, PTA-8094, or PTA-8096, respectively.

There are also provided isolated anti-DR6 monoclonal antibodies, comprising antibodies which bind to DR6 polypeptide and competitively inhibit binding of the monoclonal antibody produced by the hybridoma deposited as ATCC accession no. PTA-8095, PTA-8094, or PTA-8096 to said DR6 polypeptide. There are also provided chimeric or humanized anti-DR6 antibodies which specifically bind to DR6 polypeptide and comprise (a) a sequence derived from the 3F4.4.8, 4B6.9.7, or 1E5.5.7 antibody secreted by the hybridoma deposited with ATCC as accession number PTA-8095, PTA-8094, or PTA-8096, respectively. Optionally, such antibodies may comprise a heavy chain, light chain or variable regions derived from the 3F4.4.8, 4B6.9.7, or 1E5.5.7 antibody.

In yet another aspect, the invention concerns isolated nucleic acid molecules encoding the anti-DR6 antibodies or antibody fragments herein, vectors comprising such nucleic acid molecules, host cells comprising such nucleic acid molecules, and methods for producing antibodies and antibody fragments herein.

The invention further relates to compositions comprising DR6 antagonist(s) as herein defined, and a carrier. The carrier may be a pharmaceutically acceptable carrier, and the composition may further comprise an additional agent(s).

In an additional aspect, the invention concerns articles of manufacture comprising a container and compositions contained within said container, wherein the composition includes DR6 antagonist of the present invention. The article of manufacture may further comprise instructions for using the DR6 antagonist in vitro or in vivo. In a preferred embodiment, the instructions concern the treatment of neurological disorders.

In a related aspect, embodiments of the invention include kits comprising a first container, a label on said container, and a composition contained within said container. In such kits, the composition includes a DR6 antagonist effective for inhibiting apoptosis in at least one type of mammalian neuronal cell, the label on said container, or a package insert included in said container indicates that the composition can be used to inhibit apoptosis in at least one type of mammalian neuronal cell. Optionally the kit includes additional elements such as a second container comprising a pharmaceutically-acceptable buffer; and/or instructions for using the DR6 antagonist to inhibit apoptosis in at least one type of mammalian neuronal cell.

The invention further provides for the use of the DR6 antagonists and compositions described herein for the preparation or manufacture of a medicament for use in treating neurological disorders in mammals, including for use in treating Alzheimer's disease.

The invention also provides a method for screening for compounds that inhibit neuronal degeneration in which candidate compounds are added to a cell based assay in which a triggering event for neuronal degeration is present which would normally result in the shedding of APP from the neuronal surface. If no shedding is observed in the presence of the candidate compound, the candidate compound is an inhibitor of neuronal degeneration and may be used as a therapy for neurological diseases and disorders, such as, but not limited to Alzheimer's Disease, and for degeneration associated with injury.

### BRIEF DESCRIPTION OF THE FIGURES

FIG. 1A shows the nucleotide sequence of human DR6 cDNA (FIG. 1A-1, SEQ ID NO: 2), its derived amino acid sequence (FIG. 1A-2, SEQ ID NO:1) as well as a schematic of its domain architecture (FIG. 1A-3). In the DR6 schematic, domain boundaries including the putative signal peptide, cysteine rich domain motifs, transmembrane domain, and Death Domain are indicated. In this schematic, putative domain boundaries of the putative signal peptide, cysteine rich domain motifs, transmembrane domain, and Death Domain are indicated. FIG. 1B shows the nucleotide sequence of the 695 isoform of human amyloid precursor protein (APP) cDNA (FIG. 1B-1, SEQ ID NO: 5) and its derived amino acid sequence (FIG. 1B-2, SEQ ID NO: 6). FIG. 1C shows the amino acid sequence of the 751 isoform of human amyloid precursor protein (SEQ ID NO: 7). FIG. 1D shows the nucleotide sequence of the 770 isoform of human amyloid precursor protein (APP) cDNA (FIG. 1D-1, SEQ ID NO: 8) and its derived amino acid sequence (FIG. 1D-2, SEQ ID NO:9). See, e.g. UniProtKB/Swiss-prot entry P05067 and associated disclosure including that relating to Isoform ID P05067-1, Isoform ID P05067-4 and Isoform ID P05067-8, respectively (http://expasy.org/uniprot/P05067).

FIG. 2A shows that DR6 is strongly expressed in the developing central nervous system, including motor and commissural neurons of spinal cord and dorsal root ganglion neurons, at developmental stages E10.5-E12.5. FIG. 2B shows DR6 protein expressed on axons and cell bodies. FIG. 2C shows DR6 mRNA expressed in differentiating neurons.

FIG. 3 shows a schematic representation of axonal degeneration and neuronal cell death in a dorsal spinal cord explant survival assay; introduction of RNA interfering siRNA agents along with GFP-expressing plasmid into embryonic commissural neurons by electroporation is indicated.

FIG. 4A illustrates that inhibition of DR6 expression by small interfering RNAs blocks commissural axon degeneration and prevents neuronal cell death in the dorsal spinal cord survival assay. FIG. 4B shows an RNAi-resistant DR6 cDNA rescuing the degeneration phenotypes blocked by DR6 siRNA.

FIG. 5 shows that antagonistic DR6 antibodies helped block axonal degeneration and neuronal cell death in the dorsal spinal cord survival assay.

FIG. 6 provides a mechanistic schematic and photographs of neurons showing the down-regulation of intracellular signaling downstream of DR6 by pharmacological inhibition of c-Jun N-terminal kinase (JNK) prevents axonal degeneration and neuronal cell death in the explant survival assay.

FIG. 7 shows the neuro-protective effects of antagonistic DR6 antibodies on survival of spinal motor and interneurons in ex vivo whole embryo culture.

FIG. 8 provides photographs of E15.5 cervical spinal cord sections immunostained with cleaved Caspase 3 antibody to show that loss of DR6 results in the decrease of neuronal cell death in spinal cord and in Dorsal Root Ganglions of DR6 null embryos.

FIG. 9A shows a quantification of neuronal cells from in E15.5 DR6 KO embryos expressing cleaved caspase-3 which demonstrates an approximately 50% reduction of neuronal cell death in DR6-null embryos compared to DR6+/− littermate controls (DR6 hets). FIG. 9B provides photographs of cells showing that DR6 is required for motor axon degeneration as verified with comparisons of normal and DR6 knock-out mice in the presence and absence of neurotrophic growth factors. FIG. 9C provides photographs of cells showing that injury-induced axonal degeneration is delayed in DR6 knock-out mice.

FIG. 10A provides photographs of neurons showing that anti-DR6 antibodies inhibit axon degeneration resulting from nerve growth factor (NGF) withdrawal of diverse trophic factor deprived neurons. FIG. 10B provides further photographic data from TUNEL stain visualizations of apoptotic cell bodies in commissural, sensory and motor neurons which show that anti-DR6 antibodies inhibit degeneration of diverse trophic factor deprived neurons.

FIG. 11A provides photographs of commissural neurons showing that commissural axon degeneration can be delayed by DR6-Fc. FIG. 11B provides photographs of sensory neurons showing that sensory axon degeneration induced by NGF withdrawal can be delayed by DR6-Fc.

FIG. 12A provides photographs of neurons showing the visualization of DR6 binding sites on axons using DR6-AP. FIG. 12B provides photographs of neurons in the presence and absence of NGF showing that DR6 ligand binding sites are lost from axons following NGF deprivation. FIG. 12C provides photographs of studies of BAX null sensory axons at developmental stages E12.5 showing that a Beta secretase (BACE) inhibitor can block the disappearance of DR6-AP binding sites from sensory axons following NGF withdrawal.

FIG. 13A provides photographs of data obtained from various Western blotting procedures where polypeptides from neuronal cells were probed with DR6-AP (top left) or anti-N-APP antibody (top right), as well as polypeptides: (1) selected for their ability to bind DR6; and then (2) probed with anti-N-APP antibody (bottom, “DR6-ECD pull-down”). This data identifies amyloid precursor protein (APP) as a DR6 ectodomain-associated ligand. FIG. 13B provides photographs of data obtained from various blotting experiments that allow the visualization of DR6 ligands (including APP polypeptides) in axon conditioned media probed with DR6-AP. This blotting data identifies a number of APP polypeptides including the N-terminal APP at 35 kDa as well as the C99-APP and C83/C89 APP polypeptides.

FIG. 14A provides photographs of neurons showing that shedding of the APP ectodomain occurs early on after NGF deprivation. FIG. 14B provides photographs of cells showing that the DR6 ectodomain binds APP made by cultured cells. FIG. 14C provides photographs of cells showing that DR6 is the major receptor for N-APP on sensory axons and that APP binding sites are significantly depleted in the neuronal cells of DR6 null mice. FIG. 14D provides photographs of cells showing that DR6 function-blocking antibodies disrupt the interactions between the DR6 ectodomain and N-APP.

FIG. 15A provides photographs of neurons showing that polyclonal antibody to N-terminal APP blocks axonal degeneration in a commissural axon assay. FIG. 15B provides photographs of neurons showing that polyclonal antibodies to N-terminal APP, as well as the 22C11 anti-APP monoclonal antibodies inhibit local axonal degeneration induced by NGF removal. FIG. 15C provides photographs of neurons showing that axonal degeneration that is blocked by inhibition of β-secretase (BACE) activity can be rescued by the addition of N-APP. FIG. 15D provides photographs of neurons showing that APP removal by RNAi sensitizes neuronal cells to death induced by N-APP.

FIG. 16A provides photographs of neurons showing that DR6 function is required for N-APP induced axonal degeneration, but not degeneration triggered by Abeta. FIG. 16B provides photographs of neurons showing that function blocking DR6 antibodies fail to block axonal degeneration triggered by Abeta.

FIG. 17A provides photographs of neurons showing that axonal degeneration is delayed by inhibition of JNK and upstream caspase-8 but not by the downstream caspase-3. FIG. 17B provides photographs of motor neurons from E12.5 explant cultures showing that caspase-3 functions in cell bodies, caspase-6 in axons. FIG. 17C provides photographs of sensory neurons showing that while Caspase-3 is not required for axon degeneration, BAX is. FIG. 17D provides photographs of commissural neurons showing that Caspase-3 functions in cell bodies, while caspase-6 functions in axons.

### DETAILED DESCRIPTION OF THE INVENTION

The techniques and procedures described or referenced herein are generally well understood and commonly employed using conventional methodology by those skilled in the art, such as, for example, the widely utilized molecular cloning methodologies described in Sambrook et al., MOLECULAR CLONING: A LABORATORY MANUAL 2ND. EDITION (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

Before the present methods and assays are described, it is to be understood that this invention is not limited to the particular methodology, protocols, cell lines, animal species or genera, constructs, and reagents described as such may, of course, vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “and,” and “the” include plural referents unless the context clearly dictates otherwise. Thus, for example, reference to “a genetic alteration” includes a plurality of such alterations and reference to “a probe” includes reference to one or more probes and equivalents thereof known to those skilled in the art, and so forth. All numbers recited in the specification and associated claims (e.g. amino acids 22-81, 1-354 etc.) are understood to be modified by the term “about.”

All publications mentioned herein are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further the actual publication dates may be different from those shown and require independent verification.

### I. DEFINITIONS

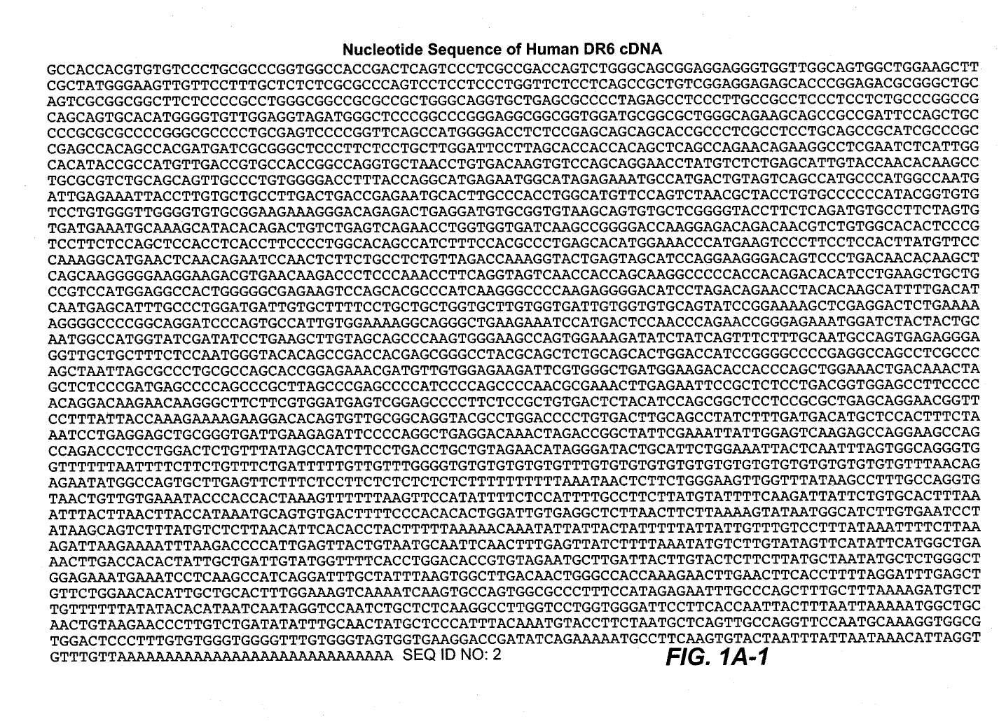

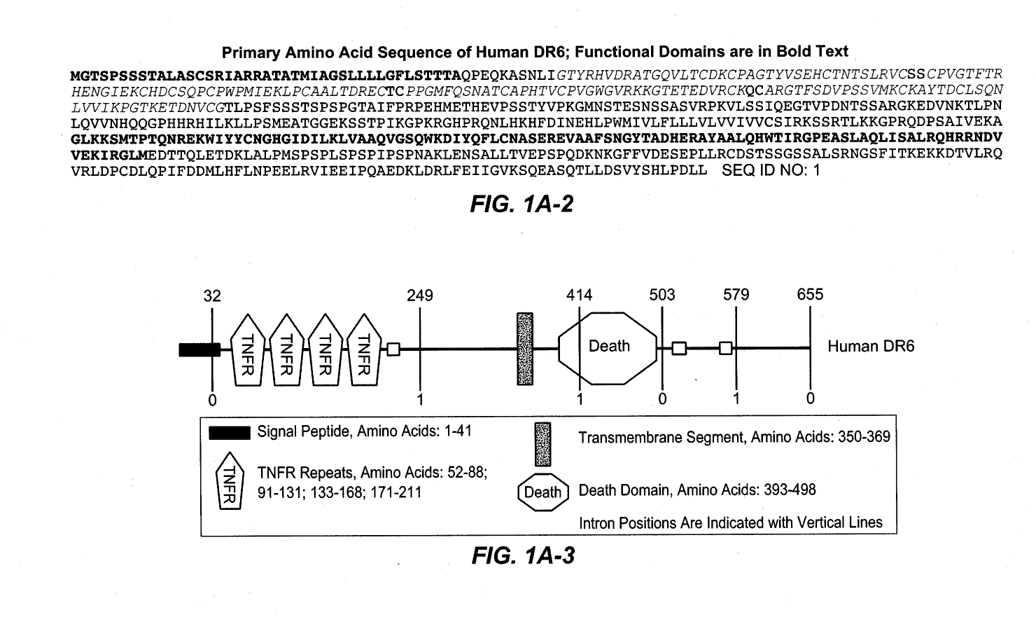

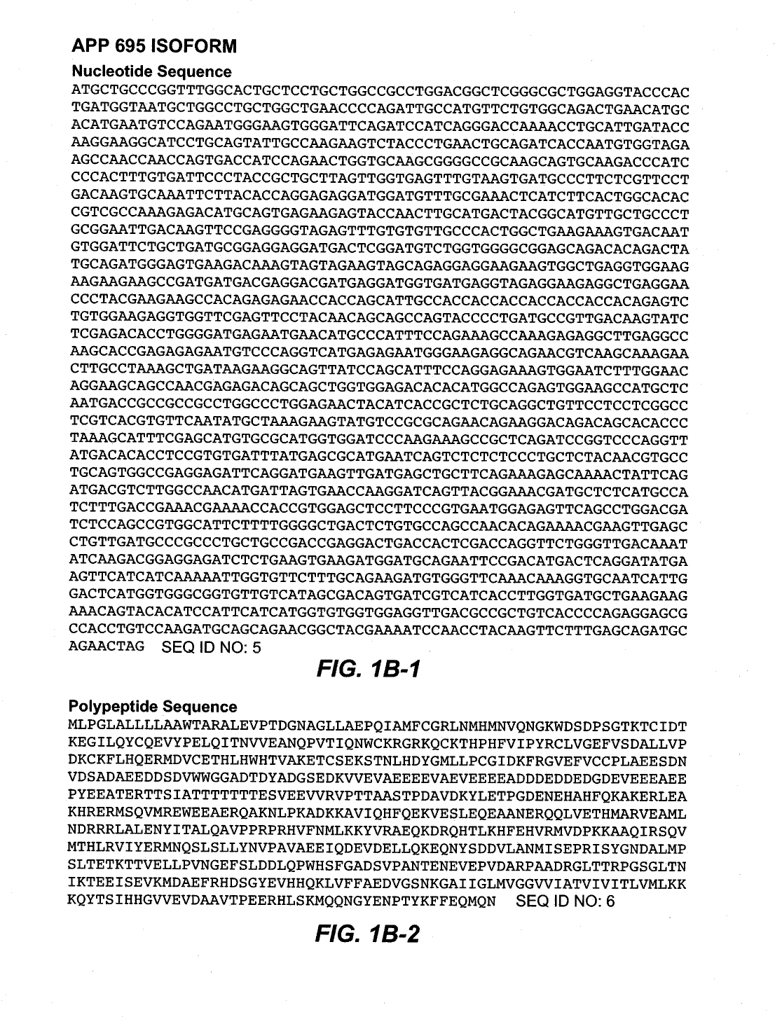

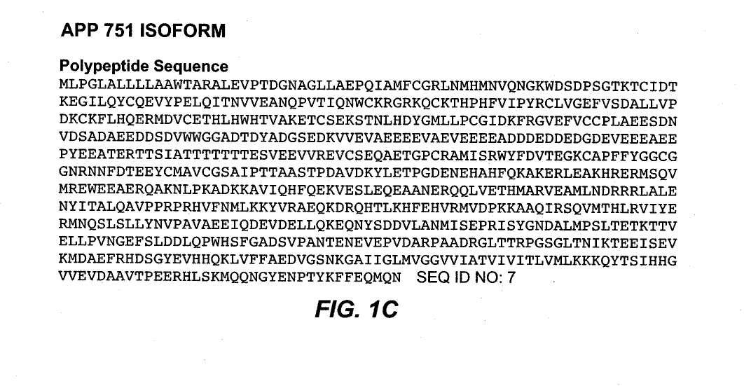

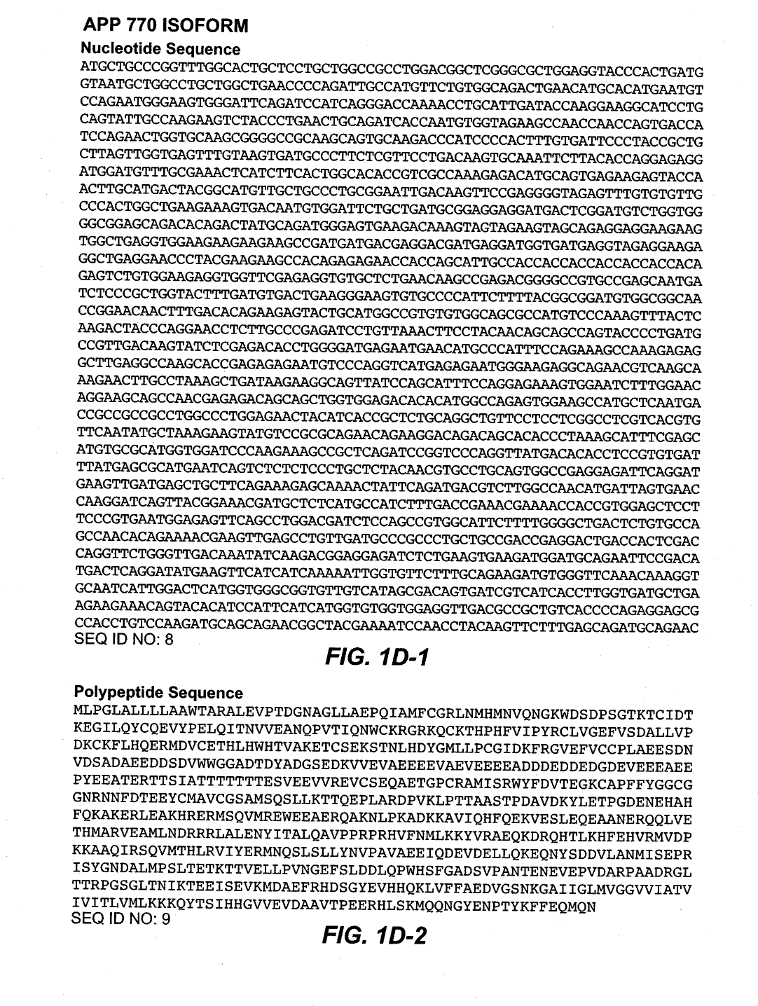

The terms “Amyloid Precursor Protein” or “APP” include the various polypeptide isoforms encoded by the APP pre-mRNA, for example the APP695, APP751 and App770 isoforms shown in FIGS. 1B-1D respectively (isoforms which are translated from alternatively spliced transcripts of the APP pre-mRNA), as well as post-translationally processed portions of APP isoforms. As is known in the art, the APP pre-mRNA transcribed from the APP gene undergoes alternative exon splicing to yield a number of isoforms (see, e.g. Sandbrink et al., Ann NY Acad. Sci. 777: 281-287 (1996); and the information associated with PubMed NCBI protein locus accession P05067). This alternative exon splicing yields three major isoforms of 695, 751, and 770 amino acids (see, e.g. Kang et al., Nature 325: 733-736 (1987); Kitaguchi et al., Nature 331: 530-532 (1988); Ponte et al., Nature 331: 525-527 (1988); and Tanzi et al., Nature 331: 528-532 (1988)). Two of these isoforms (App₇₅₁ and APP₇₇₀) contain a 56 residue insert which is highly homologous to the Kunitz family of serine protease inhibitors (KPI) and are expressed ubiquitously. In contrast, the shorter isoform lacking the KPI motif, APP₆₉₅ is expressed predominantly in the nervous system, for example in neurons and glial cells and for this reason is often termed “neuronal APP” (see, e.g. Tanzi et al., Science 235: 880-884 (1988); Neve et al., Neuron 1: 669-677 (1988); and Haas et al., J. Neurosci. 11: 3783-3793 (1991)). The APP isoforms including the 695, 751 and 770 undergo significant post-translational processing events (see, e.g. Esch et al. 1990 Science 248:1122-1124; Sisodia et al. 1990 Science 248:492-495). For example, each of these isoforms is cleaved by various secretases and/or secretase complexes, events which produce APP fragments including a N-terminal secreted polypeptides containing the APP ectodomain (sAPPα and sAPPβ). Cleavage by alpha-secretases or alternatively by beta-secretases leads to generation and extracellular release of soluble N-terminal APP polypeptides, sAPPα and sAPPβ, respectively, and the retention of corresponding membrane-anchored C-terminal fragments, C83 and C99. Subsequent processing of C83 by gamma-secretase yields P3 polypeptides. This is the major secretory pathway and is non-amyloidogenic. Alternatively, presenilin/nicastrin-mediated gamma-secretase processing of C99 releases the amyloid beta polypeptides, amyloid-beta 40 (Abeta40) and amyloid-beta 42 (Abeta42), major components of amyloid plaques, and the cytotoxic C-terminal fragments, gamma-CTF(50), gamma-CTF(57) and gamma-CTF(59). Evidence suggests that the relative importance of each cleavage event depends on the cell type. For example, non-neuronal cells preferentially process APP by α-secretase pathway(s) which cleaves APP within the Abeta sequence, thereby precluding the formation of Abeta (see, e.g. Esch et al. 1990 Science 248:1122-1124; Sisodia et al. 1990 Science 248:492-495). In contrast, neuronal cells process a much larger portion of APP₆₉₅ by β-secretase pathway(s), which generates intact Abeta by the combined activity of at least two enzyme classes. In neuronal cells the β-secretase(s) cleaves APP₆₉₅ at the amino terminus of the Abeta domain releasing a distinct N-terminal fragment (sAPPβ). In addition, γ-secretase(s) cleaves APP at alternative sites of the carboxy terminus generating species of Abeta that are either 40 (Abeta₄₀) or 42 amino acids long (Abeta₄₂) (see, e.g. Seubert et al. 1993 Nature 361:260-263; Suzuki et al. 1994 Science 264:1336-1340; and Turner et al. 1996 J. Biol. Chem. 271:8966-8970).

The terms “APP,” “APP protein” and “APP polypeptide” when used herein encompasses native APP sequences and APP variants and processed fragments thereof. These terms encompass APP expressed in a variety of mammals, including humans. APP may be endogenously expressed as occurs naturally in a variety of human tissue lineages, or may be expressed by recombinant or synthetic methods. A “native sequence APP” comprises a polypeptide having the same amino acid sequence as an APP derived from nature (e.g. the 695, 751 and 770 isoforms or processed portions thereof). Thus, a native sequence APP can have the amino acid sequence of naturally occurring APP from any mammal, including humans. Such native sequence APP can be isolated from nature or can be produced by recombinant or synthetic means. The term “native sequence APP” specifically encompasses naturally occurring processed and/or secreted forms of the (e.g., a soluble form containing, for instance, an extracellular domain sequence), naturally occurring variant forms (e.g., alternatively spliced and/or proteolytically processed forms) and naturally occurring allelic variants. APP variants may include fragments or deletion mutants of the native sequence APP.

APP polypeptides useful in embodiments of the invention include those described above and the following non-limiting examples. These illustrative forms can be selected for use in various embodiments of the invention. In some embodiments of the invention, the APP polypeptide comprises a full length APP isoform such as the APP₆₉₅ and/or APP₇₅₁ and/or APP₇₇₀ isoforms shown in FIGS. 1B-1D. In other embodiments of the invention, the APP polypeptide comprises a post-translationally processed isoform of APP, for example an APP polypeptide that has undergone cleavage by a secretase such as an α-secretase, a β-secretase or a γ-secretase (e.g. a soluble N-terminal fragment such as a sAPPα or a sAPPβ). In related embodiments of the invention, the APP polypeptide can be selected to comprise one or more specific domains such as an N-terminal ectodomain, (see, e.g. Quast et al., FASEB J. 2003; 17(12):1739-41), a heparin binding domain (see, e.g. Rossjohn et al., Nat. Struct. Biol. 1999 April; 6(4):327-31), a copper type II (see, e.g. Hesse et al., FEBS Letters 349(1): 109-116 (1994)) or a Kunitz protease inhibitor domain (see, e.g. Ponte et al., Nature; 331(6156):525-7 (1988)). In some embodiments of the invention, the APP polypeptide includes a sequence observed to comprise an epitope recognized by a DR6 antagonist disclosed herein such as an antibody or DR6 immunoadhesin, for example amino acids 22-81 of APP₆₉₅, a sequence comprising the epitope bound by monoclonal antibody 22C11 (see, e.g. Hilbich et al., J. Biol. Chem. 268(35): 26571-26577 (1993)).

In certain embodiments of the invention, the APP polypeptide does not comprise one or more specific domains or sequences, for example an APP polypeptide that does not include certain N-terminal or C-terminal amino acids (e.g. the human recombinant N-APP polypeptide disclosed in Example 12), an APP polypeptide that does not include the Kunitz protease inhibitor domain (e.g. APP₆₉₅), or an APP polypeptide that does not include Alzheimer's beta amyloid protein (Abeta) sequences (e.g. sAPPβ, a polypeptide which does not include the Aβ₄₀ and/or Aβ₄₂ sequences) (see, e.g. Bond et al., J. Struct Biol. 2003 February; 141(2):156-70). In other embodiments of the invention, an APP polypeptide used in embodiments of the invention comprises one or more domains or sequences but not other domains or sequences, for example an APP polypeptide that comprises an N-terminal ectodomain (or at least a portion thereof observed to be bound by a DR6 antagonist such as monoclonal antibody 22C11) but not a domain or sequence that is C-terminal to one or more secretase cleavage sites such as a beta amyloid (Abeta) sequence (e.g. a sAPPα or a sAPPβ).

The term “extracellular domain” “ectodomain” or “ECD” refers to a form of APP, which is essentially free of transmembrane and cytoplasmic domains. Ordinarily, the soluble ECD will have less than 1% of such transmembrane and cytoplasmic domains, and preferably, will have less than 0.5% of such domains. It will be understood that any transmembrane domain(s) identified for the polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified. In preferred embodiments, the ECD will consist of a soluble, extracellular domain sequence of the polypeptide which is free of the transmembrane and cytoplasmic or intracellular domains (and is not membrane bound).

The term “APP variant” means a APP polypeptide as defined below having at least about 80%, preferably at least about 85%, 86%, 87%, 88%, 89%, more preferably at least about 90%, 91%, 92%, 93%, 94%, most preferably at least about 95%, 96%, 97%, 98%, or 99% amino acid sequence identity with a human APP having an amino acid sequence shown in FIG. 1B-1D, or a soluble fragment thereof, or a soluble extracellular domain thereof. Such variants include, for instance, APP polypeptides wherein one or more amino acid residues are added to, or deleted from, the N- or C-terminus of the full-length or mature sequences of FIG. 1B-1D, or APP polypeptides wherein one or more amino acid residues are inserted or deleted from the internal sequence or domains of the polypeptide, including variants from other species, but excludes a native-sequence APP polypeptide

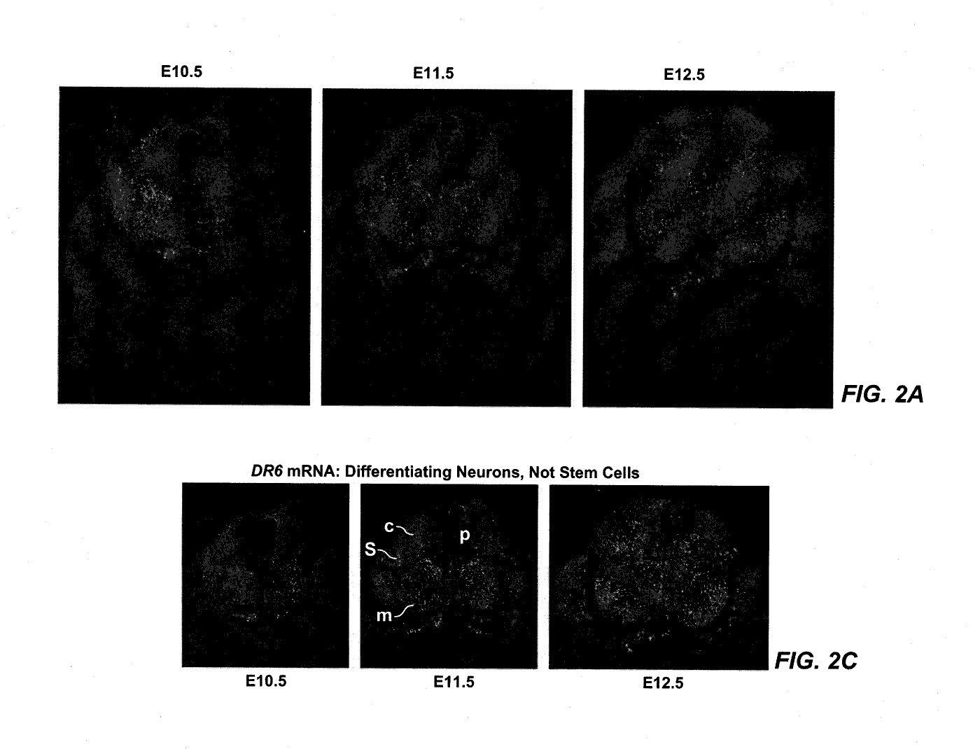

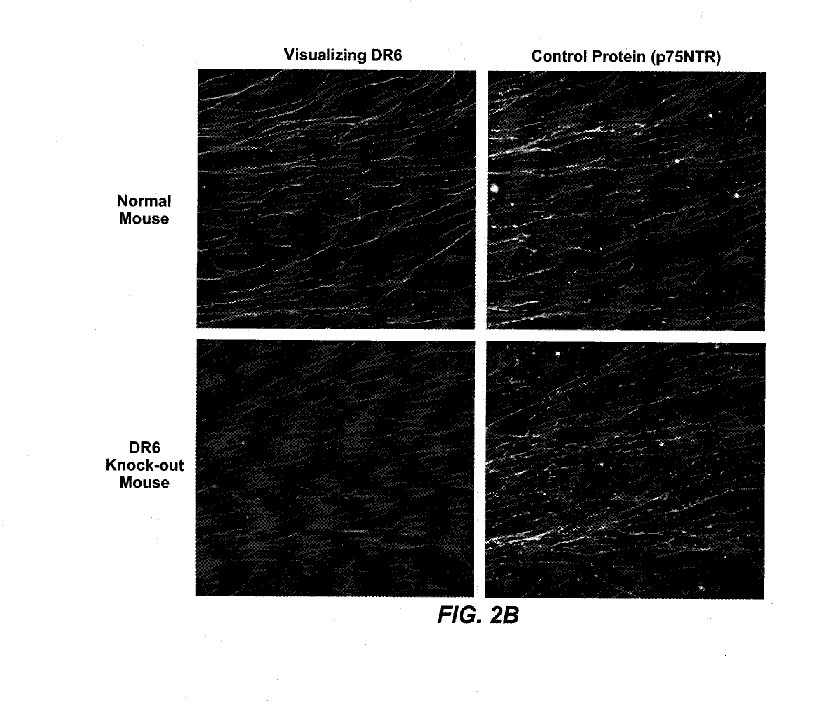

“DR6” or “DR6 receptor” includes the receptors referred to in the art whose polynucleotide and polypeptide sequences are shown in FIG. 1A-1-1A-2. Pan et al. have described the polynucleotide and polypeptide sequences for the TNF receptor family member referred to as “DR6” or “TR9” (Pan et al., FEBS Lett., 431:351-356 (1998); see also U.S. Pat. Nos. 6,358,508; 6,667,390; 6,919,078; 6,949,358). The human DR6 receptor is a 655 amino acid protein (see FIG. 1A-2) having a putative signal sequence (amino acids 1-41), an extracellular domain (amino acids 42-349), a transmembrane domain (amino acids 350-369), followed by a cytoplasmic domain (amino acids 370-655). The term “DR6 receptor” when used herein encompasses native sequence receptor and receptor variants. These terms encompass DR6 receptor expressed in a variety of mammals, including humans. DR6 receptor may be endogenously expressed as occurs naturally in a variety of human tissue lineages, or may be expressed by recombinant or synthetic methods. A “native sequence DR6 receptor” comprises a polypeptide having the same amino acid sequence as a DR6 receptor derived from nature. Thus, a native sequence DR6 receptor can have the amino acid sequence of naturally occurring DR6 receptor from any mammal, including humans. Such native sequence DR6 receptor can be isolated from nature or can be produced by recombinant or synthetic means. The term “native sequence DR6 receptor” specifically encompasses naturally occurring truncated or secreted forms of the receptor (e.g., a soluble form containing, for instance, an extracellular domain sequence), naturally occurring variant forms (e.g., alternatively spliced forms) and naturally occurring allelic variants. Receptor variants may include fragments or deletion mutants of the native sequence DR6 receptor.

The term “extracellular domain” or “ECD” refers to a form of DR6 receptor, which is essentially free of transmembrane and cytoplasmic domains. Ordinarily, the soluble ECD will have less than 1% of such transmembrane and cytoplasmic domains, and preferably, will have less than 0.5% of such domains. It will be understood that any transmembrane domain(s) identified for the polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified. In preferred embodiments, the ECD will consist of a soluble, extracellular domain sequence of the polypeptide which is free of the transmembrane and cytoplasmic or intracellular domains (and is not membrane bound).

The term “DR6 variant” means a DR6 polypeptide as defined below having at least about 80%, preferably at least about 85%, 86%, 87%, 88%, 89%, more preferably at least about 90%, 91%, 92%, 93%, 94%, most preferably at least about 95%, 96%, 97%, 98%, or 99% amino acid sequence identity with human DR6 having the deduced amino acid sequence shown in FIG. 1A, or a soluble fragment thereof, or a soluble extracellular domain thereof. Such variants include, for instance, DR6 polypeptides wherein one or more amino acid residues are added to, or deleted from, the N- or C-terminus of the full-length or mature sequences of FIG. 1A, or DR6 polypeptides wherein one or more amino acid residues are inserted or deleted from the internal sequence or domains of the polypeptide, including variants from other species, but excludes a native-sequence DR6 polypeptide. Optionally, the DR6 variant comprises a soluble form of the DR6 receptor comprising amino acids 1-349 or 42-349 of FIG. 1A with up to 10 conservative amino acid substitutions. Preferably such a variant acts as a DR6 antagonist, as defined below.

The term “DR6 antagonist” is used in the broadest sense, and includes any molecule that partially or fully blocks, inhibits, or neutralizes the ability of DR6 receptor to bind its cognate ligand, preferably, its cognate ligand APP, or to activate one or more intracellular signal(s) or intracellular signaling pathway(s) in neuronal cells or tissue, either in vitro, in situ, in vivo or ex vivo. By way of example, a DR6 antagonist may partially or fully block, inhibit, or neutralize the ability of DR6 receptor to activate one or more intracellular signal(s) or intracellular signaling pathway(s) in neuronal cells or tissue that results in apoptosis or cell death in the neuronal cells or tissue. The DR6 antagonist may act to partially or fully block, inhibit, or neutralize DR6 by a variety of mechanisms, including but not limited to, by blocking, inhibiting, or neutralizing binding of cognate ligand to DR6, formation of a complex between DR6 and its cognate ligand (e.g. APP), oligomerization of DR6 receptors, formation of a complex between DR6 receptor and heterologous co-receptor, binding of a cognate ligand to DR6 receptor/heterologous co-receptor complex, or formation of a complex between DR6 receptor, heterologous co-receptor, and its cognate ligand. DR6 antagonists may function in a direct or indirect manner. DR6 antagonists contemplated by the invention include but are not limited to, APP antibodies, DR6 antibodies, immunoadhesins, DR6 immunoadhesins, DR6 fusion proteins, covalently modified forms of DR6, DR6 variants and fusion proteins thereof, or higher oligomer forms of DR6 (dimers, aggregates) or homo- or heteropolymer forms of DR6, small molecules such as pharmacological inhibitors of the JNK signaling cascade, including small molecule and peptide inhibitors of Jun N-terminal kinase JNK activity, pharmacological inhibitors of protein kinases MLKs and MKKs activities that function upstream of JNK in the signal transduction pathway, pharmacological inhibitors of binding of JNK to scaffold protein JIP-1, pharmacological inhibitors of binding of JNK to its substrates such as c-Jun or AP-1 transcription factor complexes, pharmacological inhibitors of JNK-mediated phosphorylation of its substrates such as JNK binding domain (JBD) peptide and/or substrate binding domain of JNK and/or peptide inhibitor comprising JNK substrate phosphorylation site, small molecules that block ATP binding to JNK, and small molecules that block substrate binding to JNK.

To determine whether a DR6 antagonist partially or fully blocks, inhibits or neutralizes the ability of DR6 receptor to activate one or more intracellular signal(s) or intracellular signaling pathway(s) in neuronal cells or tissue, assays may be conducted to assess the effect(s) of the DR6 antagonist on, for example, various neuronal cells or tissues (as described in the Examples) as well as in in vivo models of stroke/cerebral ischemia, in vivo models of neurodegenerative diseases, such as mouse models of Parkinson's disease; mouse models of Alzheimer's disease; mouse models of amyotrophic lateral sclerosis ALS; mouse models of spinal muscular atrophy SMA; mouse/rat models of focal and global cerebral ischemia, for instance, common carotid artery occlusion model or middle cerebral artery occlusion models; or in ex vivo whole embryo cultures. The various assays may be conducted in known in vitro or in vivo assay formats, such as described below or as known in the art and described in the literature (See, e.g., McGowan et al., Trends in Genetics, 22:281-289 (2006); Fleming et al., NeuroRx, 2:495-503 (2005); Wong et al., Nature Neuroscience, 5:633-639 (2002)). One embodiment of an assay to determine whether a DR6 antagonist partially or fully blocks, inhibits or neutralizes the ability of DR6 receptor to activate one or more intracellular signal(s) or intracellular signaling pathway(s) in neuronal cells or tissue, comprises combining DR6 and APP in the presence or absence of a DR6 antagonist or potential DR6 antagonist (i.e. a molecule of interest); and then detecting inhibition of binding of DR6 to APP in the presence of this DR6 antagonist or potential DR6 antagonist.

By “nucleic acid” is meant to include any DNA or RNA. For example, chromosomal, mitochondrial, viral and/or bacterial nucleic acid present in tissue sample. The term “nucleic acid” encompasses either or both strands of a double stranded nucleic acid molecule and includes any fragment or portion of an intact nucleic acid molecule.

By “gene” is meant any nucleic acid sequence or portion thereof with a functional role in encoding or transcribing a protein or regulating other gene expression. The gene may consist of all the nucleic acids responsible for encoding a functional protein or only a portion of the nucleic acids responsible for encoding or expressing a protein. The nucleic acid sequence may contain a genetic abnormality within exons, introns, initiation or termination regions, promoter sequences, other regulatory sequences or unique adjacent regions to the gene.

The terms “amino acid” and “amino acids” refer to all naturally occurring L-alpha-amino acids. This definition is meant to include norleucine, ornithine, and homocysteine. The amino acids are identified by either the single-letter or three-letter designations:

| | | | | | |
|---|---|---|---|---|---|
| Asp | D | aspartic acid | Ile | I | isoleucine |
| Thr | T | threonine | Leu | L | leucine |
| Ser | S | serine | Tyr | Y | tyrosine |
| Glu | E | glutamic acid | Phe | F | phenylalanine |
| Pro | P | proline | His | H | histidine |
| Gly | G | glycine | Lys | K | lysine |
| Ala | A | alanine | Arg | R | arginine |
| Cys | C | cysteine | Trp | W | tryptophan |
| Val | V | valine | Gln | Q | glutamine |
| Met | M | methionine | Asn | N | asparagine |

In the Figures, certain other single-letter or three-letter designations may be employed to refer to and identify two or more amino acids or nucleotides at a given position in the sequence.

“Isolated,” when used to describe the various peptides or proteins disclosed herein, means peptide or protein that has been identified and separated and/or recovered from a component of its natural environment. Contaminant components of its natural environment are materials that would typically interfere with diagnostic or therapeutic uses for the peptide or protein, and may include enzymes, hormones, and other proteinaceous or non-proteinaceous solutes. In preferred embodiments, the peptide or protein will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain, or (3) to homogeneity by mass spectroscopic or peptide mapping techniques. Isolated material includes peptide or protein in situ within recombinant cells, since at least one component of its natural environment will not be present. Ordinarily, however, isolated peptide or protein will be prepared by at least one purification step.

“Percent (%) amino acid sequence identity” with respect to the sequences identified herein is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art can determine appropriate parameters for measuring alignment, including assigning algorithms needed to achieve maximal alignment over the full-length sequences being compared. For purposes herein, percent amino acid identity values can be obtained using the sequence comparison computer program, ALIGN-2, which was authored by Genentech, Inc. and the source code of which has been filed with user documentation in the US Copyright Office, Washington, D.C., 20559, registered under the US Copyright Registration No. TXU510087. The ALIGN-2 program is publicly available through Genentech, Inc., South San Francisco, Calif. All sequence comparison parameters are set by the ALIGN-2 program and do not vary.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperature. The higher the degree of desired identity between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., CURRENT PROTOCOLS IN MOLECULAR BIOLOGY, Wiley Interscience Publishers, (1995).

“High stringency conditions,” as defined herein, are identified by those that: (1) employ low ionic strength and high temperature for washing; 0.015 M sodium chloride/0.0015 M sodium citrate/0.1% sodium dodecyl sulfate at 50° C.; (2) employ during hybridization a denaturing agent; 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 ng/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., MOLECULAR CLONING: A LABORATORY MANUAL, New York: Cold Spring Harbor Press, 1989, and include overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “primer” or “primers” refers to oligonucleotide sequences that hybridize to a complementary RNA or DNA target polynucleotide and serve as the starting points for the stepwise synthesis of a polynucleotide from mononucleotides by the action of a nucleotidyltransferase, as occurs for example in a polymerase chain reaction.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The word “label” when used herein refers to a compound or composition which is conjugated or fused directly or indirectly to a reagent such as a nucleic acid probe or an antibody and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which is detectable.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

“DR6 receptor antibody,” “DR6 antibody,” or “anti-DR6 antibody” is used in a broad sense to refer to antibodies that bind to at least one form of a DR6 receptor, preferably a human DR6 receptor, such as the DR6 sequence shown in FIG. 1A or an extracellular domain sequence thereof. Optionally the DR6 antibody is fused or linked to a heterologous sequence or molecule. Preferably the heterologous sequence allows or assists the antibody to form higher order or oligomeric complexes. The term “anti-DR6 antibody” and its grammatical equivalents specifically encompass the DR6 monoclonal antibodies described in the Examples section below. Optionally, the DR6 antibody binds to DR6 receptor but does not bind or cross-react with any additional receptor of the tumor necrosis factor family (e.g. DR4, DR5, TNFR1, TNFR2, Fas). Optionally, the DR6 antibody of the invention binds to a DR6 receptor at a concentration range of about 0.067 nM to about 0.033 μM as measured in a BIAcore binding assay.

The terms “anti-APP antibody,” “APP antibody” and grammatical equivalents are used in a broad sense and refer to antibodies that bind to at least one form of APP, preferably a human APP such as the APP polypeptides isoforms specifically described herein. Preferably, the APP antibody is a DR6 antagonist antibody. For example, in methods for making and/or identifying DR6 antagonists as disclosed herein, one or more isoforms of APP and/or a portion thereof can be used as an immunogen to immunize an animal (e.g. a mouse as part of a process for generating a monoclonal antibody) and/or as a probe to screen a library of compounds (e.g. a recombinant antibody library). Typical APP polypeptides useful in embodiments of the invention include the following non-limiting examples. These illustrative forms can be selected for use in various embodiments of the invention. In some embodiments of the invention, the APP polypeptide comprises a full length APP isoform such as the APP₆₉₅ and/or APP₇₅₁ and/or APP₇₇₀ isoforms shown in FIG. 1. In other embodiments of the invention, the APP polypeptide comprises a post-translationally processed isoform of APP, for example an APP polypeptide that has undergone cleavage by a secretase such as an α-secretase, a β-secretase or a γ-secretase (e.g. a soluble N-terminal fragment such as a sAPPα or a sAPPβ). In related embodiments of the invention, the APP polypeptide can be selected to comprise one or more specific domains such as an N-terminal ectodomain, (see, e.g. Quast et al., FASEB J. 2003; 17(12):1739-41), a heparin binding domain (see, e.g. Rossjohn et al., Nat. Struct. Biol. 1999 April; 6(4):327-31), a copper type II (see, e.g. Hesse et al., FEBS Letters 349(1): 109-116 (1994)) or a Kunitz protease inhibitor domain (see, e.g. Ponte et al., Nature; 331(6156):525-7 (1988)). In some embodiments of the invention, the APP polypeptide includes a sequence observed to comprise an epitope recognized by a DR6 antagonist disclosed herein such as an antibody or DR6 immunoadhesin, for example amino acids 22-81 of APP₆₉₅, a sequence comprising the epitope bound by monoclonal antibody 22C11 (see, e.g. Hilbich et al., J. Biol. Chem., 268(35): 26571-26577 (1993)). In certain embodiments of the invention, the APP polypeptide does not comprise one or more specific domains or sequences, for example an APP polypeptide that does not include certain N-terminal or C-terminal amino acids (e.g. the human recombinant N-APP polypeptide disclosed in Example 12), an APP polypeptide that does not include the Kunitz protease inhibitor domain (e.g. APP₆₉₅), or an APP polypeptide that does not include Alzheimer's beta amyloid protein (Abeta) sequences (e.g. sAPPβ, a polypeptide which does not include the Aβ₄₀ and/or Aβ₄₂ sequences) (see, e.g. Bond et al., J. Struct Biol. 2003 February; 141(2):156-70). In other embodiments of the invention, an APP polypeptide used in embodiments of the invention comprises one or more domains or sequences but not other domains or sequences, for example an APP polypeptide that comprises an N-terminal ectodomain (or at least a portion thereof observed to be bound by a DR6 antagonist such as monoclonal antibody 22C11) but not a domain or sequence that is C-terminal to one or more secretase cleavage sites such as a beta amyloid (Abeta) sequence (e.g. a sAPPα or a sAPPβ). Optionally, the anti-APP antibody will inhibit binding of the APP polypeptide to DR6 and bind to an APP polypeptide at concentrations of 10 μg/ml to 50 μg/ml, as described herein, and/or as measured in a quantitative cell-based binding assay.

The term “antibody” herein is used in the broadest sense and specifically covers intact monoclonal antibodies, polyclonal antibodies, multispecific antibodies (e.g. bispecific antibodies) formed from at least two intact antibodies, and antibody fragments so long as they exhibit the desired biological activity.

“Antibody fragments” comprise a portion of an intact antibody, preferably comprising the antigen-binding or variable region thereof. Examples of antibody fragments include Fab, Fab′, F(ab′)₂, and Fv fragments; diabodies; linear antibodies; single-chain antibody molecules; and multispecific antibodies formed from antibody fragments.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable or complementary determining regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., SEQUENCES OF PROTEINS OF IMMUNOLOGICAL INTEREST, 5th Ed. Public Health Service, National Institutes of Health, Bethesda, Md. (1991)). The constant domains are not involved directly in binding an antibody to an antigen, but exhibit various effector functions, such as participation of the antibody in antibody-dependent cell-mediated cytotoxicity (ADCC).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ) based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “hypervariable region” when used herein refers to the amino acid residues of an antibody which are responsible for antigen-binding. The hypervariable region comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined.

An antibody “which binds” an antigen of interest is one capable of binding that antigen with sufficient affinity and/or avidity such that the antibody is useful as a therapeutic or diagnostic agent for targeting a cell expressing the antigen.

For the purposes herein, “immunotherapy” will refer to a method of treating a mammal (preferably a human patient) with an antibody, wherein the antibody may be an unconjugated or “naked” antibody, or the antibody may be conjugated or fused with heterologous molecule(s) or agent(s), such as one or more cytotoxic agent(s), thereby generating an “immunoconjugate.”

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The term “tagged” when used herein refers to a chimeric molecule comprising an antibody or polypeptide fused to a “tag polypeptide”. The tag polypeptide has enough residues to provide an epitope against which an antibody can be made or to provide some other function, such as the ability to oligomerize (e.g. as occurs with peptides having leucine zipper domains), yet is short enough such that it generally does not interfere with activity of the antibody or polypeptide. The tag polypeptide preferably also is fairly unique so that a tag-specific antibody does not substantially cross-react with other epitopes. Suitable tag polypeptides generally have at least six amino acid residues and usually between about 8 to about 50 amino acid residues (preferably, between about 10 to about 20 residues).

The terms “Fc receptor” or “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and Fcγ RIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)). FcRs herein include polymorphisms such as the genetic dimorphism in the gene that encodes FcγRIIIa resulting in either a phenylalanine (F) or a valine (V) at amino acid position 158, located in the region of the receptor that binds to IgG1. The homozygous valine FcγRIIIa (FcγRIIIa-158V) has been shown to have a higher affinity for human IgG1 and mediate increased ADCC in vitro relative to homozygous phenylalanine FcγRIIIa (FcγRIIIa-158F) or heterozygous (FcγRIIIa-158F/V) receptors.

The term “polyol” when used herein refers broadly to polyhydric alcohol compounds. Polyols can be any water-soluble poly(alkylene oxide) polymer for example, and can have a linear or branched chain. Preferred polyols include those substituted at one or more hydroxyl positions with a chemical group, such as an alkyl group having between one and four carbons. Typically, the polyol is a poly(alkylene glycol), preferably poly(ethylene glycol) (PEG). However, those skilled in the art recognize that other polyols, such as, for example, poly(propylene glycol) and polyethylene-polypropylene glycol copolymers, can be employed using the techniques for conjugation described herein for PEG. The polyols include those well known in the art and those publicly available, such as from commercially available sources such as Nektar® Corporation.

The term “conjugate” is used herein according to its broadest definition to mean joined or linked together. Molecules are “conjugated” when they act or operate as if joined.

The expression “effective amount” refers to an amount of an agent (e.g. DR6 antagonist etc.) which is effective for preventing, ameliorating or treating the disorder or condition in question. It is contemplated that the DR6 antagonists of the invention will be useful in slowing down, or stopping, progression of degenerative neurological disorders or in enhancing repair of damaged neuronal cells or tissue and assist in restoring proper nerve function.

The terms “treating,” “treatment” and “therapy” as used herein refer to curative therapy, prophylactic therapy, and preventative therapy. Consecutive treatment or administration refers to treatment on at least a daily basis without interruption in treatment by one or more days. Intermittent treatment or administration, or treatment or administration in an intermittent fashion, refers to treatment that is not consecutive, but rather cyclic in nature.

As used herein, the term “disorder” in general refers to any condition that would benefit from treatment with the DR6 antagonists described herein. This includes chronic and acute disorders, as well as those pathological conditions which predispose the mammal to the disorder in question.

“Neuronal cells or tissue” refers generally to motor neurons, interneurons including but not limited to commissural neurons, sensory neurons including but not limited to dorsal root ganglion neurons, dopamine (DA) neurons of substantia nigra, striatal DA neurons, cortical neurons, brainstem neurons, spinal cord interneurons and motor neurons, hippocampal neurons including but not limited to CA1 pyramidal neurons of the hippocampus, and forebrain neurons. The term neuronal cells or tissue is intended herein to refer to neuronal cells consisting of a cell body, axon(s) and dendrite(s), as well as to axon(s) or dendrite(s) that may form part of such neuronal cells.

“Neurological disorder” is used herein to refer to conditions that include neurodegenerative conditions, neuronal cell or tissue injuries characterized by dysfunction of the central or peripheral nervous system or by necrosis and/or apoptosis of neuronal cells or tissue, and neuronal cell or tissue damage associated with trophic factor deprivation. Examples of neurodegenerative diseases include familial and sporadic amyotrophic lateral sclerosis (FALS and ALS, respectively), familial and sporadic Parkinson's disease, Huntington's disease (Huntington's chorea), familial and sporadic Alzheimer's disease, Spinal Muscular Atrophy (SMA), optical neuropathies such as glaucoma or associated disease involving retinal degeneration, diabetic neuropathy, or macular degeneration, hearing loss due to degeneration of inner ear sensory cells or neurons, epilepsy, Bell's palsy, frontotemporal dementia with parkinsonism linked to chromosome 17 (FTDP-17), multiple sclerosis, diffuse cerebral corical atrophy, Lewy-body dementia, Pick disease, trinucleotide repeat disease, prion disorder, and Shy-Drager syndrome. Injury or damage of neuronal cells or tissue may occur from a variety of different causes that compromise the survival or proper function of neuronal cells or tissue, including but not limited to: acute and non-acute injury from, e.g., ischemic conditions restricting (temporarily or permanently) blood flow as in global and focal cerebral ischemia (stroke); incisions or cuts for instance to cerebral tissue or spinal cord; lesions or placques in neuronal tissues; deprivation of trophic factor(s) needed for growth and survival of cells; exposure to neurotoxins such as chemotherapeutic agents; as well as incidental to other disease states such as chronic metabolic diseases such as diabetes or renal dysfunction.

By “subject” or “patient” is meant any single subject for which therapy is desired, including humans. Also intended to be included as a subject are any subjects involved in clinical research trials not showing any clinical sign of disease, or subjects involved in epidemiological studies, or subjects used as controls.

The term “mammal” as used herein refers to any mammal classified as a mammal, including humans, cows, horses, dogs and cats. In a preferred embodiment of the invention, the mammal is a human.

### II. EXEMPLARY METHODS AND MATERIALS OF THE INVENTION

Previous studies have examined the phenomenon of cell death during development of the nervous system (Hamburger et al., J. Neurosci., 1:60-71 (1981); Oppenheim, Ann. Rev. Neurosci., 14:453-501 (1991); O'Leary et al., J. Neurosci., 6:3692-3705 (1986); Henderson et al., Nature, 363:266-270 (1993); Yuen et al., Brain Dev., 18:362-368 (1996)). It is believed that death of neuronal cells plays a role in the development of and/or progression of various neurological disorders, such as familial and sporadic amyotrophic lateral sclerosis (FALS and ALS, respectively), familial and sporadic Parkinson's disease, Huntington's disease, familial and sporadic Alzheimer's disease and Spinal Muscular Atrophy (SMA) (Price et al., Science, 282:1079-1083 (1998)).

Applicants surprisingly found that DR6, a member of the TNFR family, is highly expressed in embryonic and adult central nervous system, including cerebral cortex, hippocampus, motor neurons and interneurons of the spinal cord. As described in the Examples below, Applicants conducted various experimental assays to examine the role DR6 may play as a regulator of neuronal cell survival or death. Commissural neurons are dependent for their survival on trophic support from one of their intermediate targets, the floorplate of the spinal cord. In explant cultures in vitro, Applicants found that inhibition of DR6 expression by RNA interference blocked axonal degeneration of the commissural neurons. Anti-DR6 monoclonal antibodies were also tested in dorsal spinal cord survival assays, and it was determined that inhibition of DR6 receptor signaling by DR6-specific antibodies 3F4.4.8; 4B6.9.7; and 1E5.5.7 prevented axonal degeneration of commissural neurons in explant cultures in vitro. DR6 has been reported in the literature to signal through activation of JNK (Pan et al., supra 1998; Zhao et al., supra 2001). Accordingly, to investigate roles of DR6-JNK signaling in axonal degeneration, dorsal spinal cord survival assays were conducted wherein the JNK signaling pathway in commissural neurons was blocked by a peptide inhibitor. This inhibition of JNK signaling partially blocked axonal degeneration in the dorsal spinal cord survival assays. Thus, it is believed that DR6 signals degeneration of axonal processes at least in part through the JNK pathway. To better understand physiological roles of DR6 in the regulation of neuronal cell death in development, DR6 signaling was blocked by anti-DR6 antibodies in a whole embryo culture system. Strikingly, inhibition of DR6 signaling by certain DR6-specific antibodies protected spinal cord neurons against naturally occurring developmental cell death in this system. Therefore, DR6 antagonists, such as DR6 antagonist antibodies, may be utilized to reduce neuronal cell death that occurs in neurological disorders such as neurodegenerative diseases (e.g. ALS, SMA, Alzheimer's, and Parkinson's diseases, FTDP-17, Huntington's disease) and stroke. To examine whether DR6 functions as a bona fide pro-apoptotic receptor in vivo, Applicants analyzed phenotypes of DR6 knockout embryos at developmental stage E15.5. In line with the proposed roles of DR6 as a negative regulator of neuronal cell survival, an approximately 40% to 50% reduction in neuronal cell death was detected in DR6 null spinal cords and dorsal root ganglions as compared to DR6 heterozygous littermate controls.

Applicants have also surprisingly found that amyloid precursor protein (APP) is a cognate ligand of DR6 receptor and further that APP functions to trigger axonal degeneration via the DR6 receptor. Amyloid precursor protein has previously been hypothesized to play some, though not fully understood, role in Alzheimer's disease (Selkoe, J. Biol. Chem. 271:18295 (1996); Scheuner; et al., Nature Med. 2:864 (1996); Goate, et al., Nature 349:704 (1991)).

It is believed that DR6 antagonists will be particularly useful in treating various neurological disorders. The present invention accordingly provides DR6 antagonist compositions and methods for inhibiting, blocking or neutralizing DR6 activity in a mammal which comprise administration of an effective amount of DR6 antagonist. Preferably, the amount of DR6 antagonist employed will be an amount effective to block axonal degeneration and neuronal cell death. This can be accomplished in accordance, for instance, with the methods described below and in the Examples.

The DR6 antagonists which can be employed in the methods include, but are not limited to, DR6 and/or APP immunoadhesins, fusion proteins comprising DR6 and/or APP, covalently modified forms of DR6 and/or APP, DR6 and/or APP variants, fusion proteins thereof, and DR6 and/or APP antibodies. Various techniques that can be employed for making the antagonists are described herein. For instance, methods and techniques for preparing DR6 and APP polypeptides are described. Further modifications of the DR6 and APP polypeptides, and antibodies to DR6 and APP are also described.

The invention disclosed herein has a number of embodiments. The invention provides methods of inhibiting binding of DR6 to APP comprising exposing DR6 polypeptide and/or APP polypeptide to one or more DR6 antagonists under conditions wherein binding of DR6 to APP is inhibited. Related embodiments of the invention provide methods of inhibiting binding of DR6 polypeptide comprising amino acids 1-655 of SEQ ID NO: 1 and an APP polypeptide comprising amino acids 66-81 of SEQ ID NO: 6 (e.g. sAPPβ), the method comprising combining the DR6 polypeptide and the APP polypeptide with an isolated antagonist that binds DR6 or APP, wherein the isolated antagonist is chosen from at least one of an antibody that binds APP, an antibody that binds DR6 and a soluble DR6 polypeptide comprising amino acids 1-354 of SEQ ID NO: 1; and the isolated antagonist is selected for its ability to inhibit binding of DR6 and APP; so that binding of DR6 to APP is inhibited.

Optionally in such methods, one or more of DR6 antagonists are selected from an antibody that binds DR6 (e.g. an antibody that binds DR6 competitively inhibits binding of the 3F4.4.8, 4B6.9.7, or 1E5.5.7 monoclonal antibody produced by the hybridoma cell line deposited as ATCC accession number PTA-8095, PTA-8094, or PTA-8096, respectively), a soluble DR6 polypeptide comprising amino acids 1-354 of SEQ ID NO: 1 (e.g. a DR6 immunoadhesin), or an antibody that binds APP (e.g. monoclonal antibody 22C11). In certain embodiments of the invention, a DR6 antagonist is an antibody that binds DR6, antibody that binds APP or soluble DR6 polypeptide that is linked to one or more non-proteinaceous polymers selected from the group consisting of polyethylene glycol, polypropylene glycol, and polyoxyalkylene.

In optional embodiments of these methods, the DR6 polypeptide is expressed on the cell surface of one or more mammalian cells (e.g. commissural neuron cell, a sensory neuron cell or a motor neuron cell) and binding of said one or more DR6 antagonists inhibits DR6 activation or signaling. In one such embodiment of the invention, the method is performed in vitro to inhibit apoptosis in one or more mammalian cells expressing DR6 so as to enhance growth and/or regeneration and/or survival of neuronal cells in a tissue culture. By way of example, such DR6 antagonists are useful as an in vitro additive to tissue medias, for example those designed to propagate neuronal cell cultures. In particular, as is known in the art, the propagation of certain neuronal cells cultures can be problematic due to the tendency of such cells to undergo apoptosis. Some neuronal cultures, for example, die in the absence of exogenous factors such as nerve growth factor. The disclosure provided herein shows that DR6 antagonists can be used in such neuronal cell cultures to enhance cell growth and/or regeneration and/or survival, for example, in a manner akin to the use of nerve growth factor in such cultures.

In further embodiments of the invention, methods of inhibiting binding of DR6 to APP may be conducted in vivo in a mammal having a neurological condition or disorder. Optionally the neurological condition or disorder is amyotrophic lateral sclerosis, Parkinson's disease, Huntington's disease or Alzheimer's disease. Alternatively, the neurological condition or disorder comprises neuronal cell or tissue injury from stroke, trauma to cerebral or spinal cord tissue, or lesions in neuronal tissue.

Further embodiments of the invention provide methods of treating a mammal having a neurological condition or disorder, comprising administering to said mammal an effective amount of one or more DR6 antagonists. Typically in such methods, the one or more DR6 antagonists are selected from an antibody that binds DR6, a soluble DR6 polypeptide comprising amino acids 1-354 of SEQ ID NO: 1, and an antibody that binds APP. In optional embodiments of the invention, the neurological condition or disorder is amyotrophic lateral sclerosis, Parkinson's disease, Huntington's disease or Alzheimer's disease. Alternatively, the neurological condition or disorder comprises neuronal cell or tissue injury from stroke, trauma to cerebral or spinal cord tissue, or lesions in neuronal tissue. In various embodiments of the invention, one or more further therapeutic agents is administered to said mammal. In certain illustrative embodiments of the invention, the one or more further therapeutic agents are selected from NGF, an apoptosis inhibitor, an EGFR inhibitor, a β-secretase inhibitor, a γ-secretase inhibitor, a cholinesterase inhibitor, an anti-Abeta antibody and a NMDA receptor antagonist. Optionally the one or more DR6 antagonists and/or further therapeutic agents is administered to the mammal via injection, infusion or perfusion.

Yet further embodiments of the invention provide methods of identifying a molecule of interest which inhibits binding of DR6 to APP, the method comprising: combining DR6 and APP in the presence or absence of a molecule of interest; and then detecting inhibition of binding of DR6 to APP in the presence of said molecule of interest. Related embodiments of the invention provide methods of determining if a composition modulates binding between a DR6 polypeptide comprising amino acids 1-655 of SEQ ID NO: 1 (and optionally amino acids 1-354 of SEQ ID NO: 1) and APP polypeptide comprising amino acids 66-81 of SEQ ID NO: 6 (e.g. APP₆₉₅, sAPPα or sAPPβ), the method comprising combining the composition with DR6 and APP; and then comparing the binding between DR6 and APP in the presence of the composition with the binding between DR6 and APP in the absence of the composition; so as to determine if the composition modulates the binding between DR6 and APP. Optionally, differences in binding in such methods are measured via a surface plasmon resonance (SPR) technology (e.g. as is available from Biacore Life Sciences). Embodiments of the invention further include a molecule of interest that is identified in accordance with these methods.

Further embodiments of the invention include methods of diagnosing a patient with a neurological disorder or susceptible to a neurological disorder, comprising obtaining a sample from the patient and testing the sample for the presence of a DR6 polypeptide variant having a polypeptide sequence that differs from the DR6 polypeptide sequence of SEQ ID NO: 1. Optionally the methods further comprise identifying the polypeptide variant as having an affinity for an APP polypeptide that differs from the affinity observed for the DR6 polypeptide sequence of SEQ ID NO: 1. Related embodiments of the invention include methods of determining if a polypeptide variant of DR6 comprising amino acids 1-655 of SEQ ID NO: 1 is present in a mammal, the method comprising comparing the sequence of a DR6 polypeptide expressed with SEQ ID NO: 1 in the mammal so as to determine if a polypeptide variant of DR6 is present in the mammal. Certain embodiments of these methods may include the further step of identifying a polypeptide variant observed to be present in a mammal as an APP binding variant, wherein an APP binding variant is characterized as having a binding affinity for an amyloid precursor protein (APP) polypeptide comprising amino acids 66-81 of SEQ ID NO: 6 (e.g. APP₆₉₅, sAPPα or sAPPβ), that is different from the binding affinity of the DR6 polypeptide comprising SEQ ID NO: 1 for an APP polypeptide comprising amino acids 66-81 of SEQ ID NO: 6. Optionally, differences in binding affinity in such methods are measured via a surface plasmon resonance (SPR) technology (e.g. as is available from Biacore Life Sciences). Some embodiments of these methods may include the step of selecting the individual patient as one having a symptom or condition observed in amyotrophic lateral sclerosis, Parkinson's disease, Huntington's disease or Alzheimer's disease.

In addition to the full-length native sequence DR6 and APP polypeptides described herein, it is contemplated that DR6 and/or APP polypeptide variants can be prepared. DR6 and/or APP variants can be prepared by introducing appropriate nucleotide changes into the encoding DNA, and/or by synthesis of the desired polypeptide. Those skilled in the art will appreciate that amino acid changes may alter post-translational processes of the DR6 and/or APP polypeptide, such as changing the number or position of glycosylation sites or altering the membrane anchoring characteristics.

Variations in the DR6 and/or APP polypeptides described herein, can be made, for example, using any of the techniques and guidelines for conservative and non-conservative mutations set forth, for instance, in U.S. Pat. No. 5,364,934. Variations may be a substitution, deletion or insertion of one or more codons encoding the polypeptide that results in a change in the amino acid sequence as compared with the native sequence polypeptide. Optionally the variation is by substitution of at least one amino acid with any other amino acid in one or more of the domains of the DR6 and/or APP polypeptide. Guidance in determining which amino acid residue may be inserted, substituted or deleted without adversely affecting the desired activity may be found by comparing the sequence of the DR6 polypeptide with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for DR6 and/or APP antagonistic activity.

DR6 and/or APP polypeptide fragments are provided herein. Such fragments may be truncated at the N-terminus or C-terminus, or may lack internal residues, for example, when compared with a full length native protein. Certain fragments lack amino acid residues that are not essential for the desired biological activity of the DR6 polypeptide.

DR6 and/or APP polypeptide fragments may be prepared by any of a number of conventional techniques. Desired peptide fragments may be chemically synthesized. An alternative approach involves generating polypeptide fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR.

In particular embodiments, conservative substitutions of interest are shown in the Table below under the heading of preferred substitutions. If such substitutions result in a change in biological activity, then more substantial changes, denominated exemplary substitutions in the Table, or as further described below in reference to amino acid classes, are introduced and the products screened.

| Original Residue | Exemplary Substitutions | Preferred Substitutions |
|---|---|---|
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; met; ala; | ile |
| | phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; | leu |
| | norleucine | |

Substantial modifications in function or immunological identity of the DR6 and/or APP polypeptides are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis (Carter et al., Nucl. Acids Res., 13:4331 (1986); Zoller et al., Nucl. Acids Res., 10:6487 (1987)), cassette mutagenesis (Wells et al., Gene, 34:315 (1985)), restriction selection mutagenesis (Wells et al., Philos. Trans. R. Soc. London SerA, 317:415 (1986)) or other known techniques can be performed on the cloned DNA to produce the DR6 polypeptide variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant (Cunningham and Wells, Science, 244:1081-1085 (1989)). Alanine is also typically preferred because it is the most common amino acid. Further, it is frequently found in both buried and exposed positions (Creighton, THE PROTEINS, (W.H. Freeman & Co., N.Y.); Chothia, J. Mol. Biol., 150:1 (1976)). If alanine substitution does not yield adequate amounts of variant, an isoteric amino acid can be used.

Any cysteine residue not involved in maintaining the proper conformation of the DR6 and/or APP polypeptide also may be substituted, generally with serine, to improve the oxidative stability of the molecule and prevent aberrant crosslinking. Conversely, cysteine bond(s) may be added to the DR6 and/or APP polypeptide to improve its stability.

Embodiments of the invention disclosed herein apply to a wide variety of APP polypeptides. In certain embodiments of the invention for example, an APP is the full length 695, 750 or 770 APP isoform shown in FIGS. 1B-1D. In other embodiments of the invention, the APP comprises an n-terminal portion of APP having the APP ectodomain and which is which produced from a post-translational processing event (e.g. sAPPα or sAPPβ). Optionally for example, an APP can comprise a soluble form of one of 695, 750 or 770 APP isoforms that results from cleavage by a secretase, for example a soluble form of neuronal APP₆₉₅ that results from cleavage by a β-secretase. In a specific illustrative embodiment, an APP comprises amino acids 20-591 of APP₆₉₅ (see, e.g. Jin et al., J. Neurosci., 14(9): 5461-5470 (1994). In another embodiment of the invention, an APP comprises a polypeptide having the epitope recognized by monoclonal antibody 22C11 (e.g. as is available from Chemicon International Inc., Temecula, Calif., U.S.A.). Optionally, an APP comprises residues 66-81 of APP₆₉₅, a region containing the 22C11 epitope (see, e.g. Hilbrich, J. Biol. Chem. 268 (35):26571-26577 (1993)).

The description below relates primarily to production of DR6 and/or APP polypeptides by culturing cells transformed or transfected with a vector containing DR6 polypeptide-encoding nucleic acid. It is, of course, contemplated that alternative methods, which are well known in the art, may be employed to prepare DR6 and/or APP polypeptides. For instance, the appropriate amino acid sequence, or portions thereof, may be produced by direct peptide synthesis using solid-phase techniques (see, e.g., Stewart et al., SOLID-PHASE PEPTIDE SYNTHESIS, W.H. Freeman Co., San Francisco, Calif. (1969); Merrifield, J. Am. Chem. Soc., 85:2149-2154 (1963)). In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the DR6 and/or APP polypeptide may be chemically synthesized separately and combined using chemical or enzymatic methods to produce the desired DR6 and/or APP polypeptide.

The methods and techniques described are similarly applicable to production of DR6 and/or APP variants, modified forms of DR6 and/or APP and DR6 and/or APP antibodies.

Isolation of DNA Encoding DR6 and/or APP Polypeptides

DNA encoding DR6 and/or APP polypeptide may be obtained from a cDNA library prepared from tissue believed to possess the DR6 and/or APP polypeptide mRNA and to express it at a detectable level. Accordingly, human DR6 and/or APP polypeptide DNA can be conveniently obtained from a cDNA library prepared from human tissue. The DR6 and/or APP polypeptide-encoding gene may also be obtained from a genomic library or by known synthetic procedures (e.g., automated nucleic acid synthesis).

Libraries can be screened with probes (such as oligonucleotides of at least about 20-80 bases) designed to identify the gene of interest or the protein encoded by it. Screening the cDNA or genomic library with the selected probe may be conducted using standard procedures, such as described in Sambrook et al., MOLECULAR CLONING: A LABORATORY MANUAL (New York: Cold Spring Harbor Laboratory Press, 1989). An alternative means to isolate the gene encoding DR6 polypeptide is to use PCR methodology (Sambrook et al., supra; Dieffenbach et al., PCR PRIMER: A LABORATORY MANUAL (Cold Spring Harbor Laboratory Press, 1995)).

Techniques for screening a cDNA library are well known in the art. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

### Selection and Transformation of Host Cells

Host cells are transfected or transformed with expression or cloning vectors described herein for DR6 and/or APP polypeptide production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in MAMMALIAN CELL BIOTECHNOLOGY: A PRACTICAL APPROACH, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. USA, 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan^{r}; E. coli W3110 strain 37D6, which has the complete genotype tonA ptr3 phoA E15 (argF-lac)169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for DR6 polypeptide-encoding vectors. Saccharomyces cerevisiae is a commonly used lower eukaryotic host microorganism. Others include Schizosaccharomyces pombe (Beach and Nurse, Nature, 290: 140 [1981]; EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteria, 154(2):737-742 (1983)), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 (1988)); Candida; Trichoderma reesia (EP 244,234); Neurospora crassa (Case et al., Proc. Natl. Acad. Sci. USA, 76:5259-5263 (1979)); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81: 1470-1474 (1984)) and A. niger (Kelly and Hynes, EMBO J., 4:475-479 (1985)). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated DR6 and/or APP polypeptide are derived from multicellular organisms. Examples of invertebrate cells include insect cells such as Drosophila S2 and Spodoptera Sf9, as well as plant cells, such as cell cultures of cotton, corn, potato, soybean, petunia, tomato, and tobacco. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for DR6 and/or APP polypeptide production and cultured in conventional nutrient media modified as appropriate for inducing promoters, selecting transformants, or amplifying the genes encoding the desired sequences.

### Selection and Use of a Replicable Vector

The nucleic acid (e.g., cDNA or genomic DNA) encoding DR6 and/or APP polypeptide may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The DR6 and/or APP may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the DR6 and/or APP polypeptide-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the DR6 and/or APP polypeptide-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 (Stinchcomb et al., Nature, 282:39 (1979); Kingsman et al., Gene, 7:141 (1979); Tschemper et al., Gene, 10:157 (1980)). The trp1 gene provides a selection marker for a mutant strain of yeast lacking the ability to grow in tryptophan, for example, ATCC No. 44076 or PEP4-1 (Jones, Genetics, 85:12 (1977)).

Expression and cloning vectors usually contain a promoter operably linked to the DR6 and/or APP polypeptide-encoding nucleic acid sequence to direct mRNA synthesis. Promoters recognized by a variety of potential host cells are well known. Promoters suitable for use with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system (Goeddel, Nucl. Acids Res., 8:4057 (1980); EP 36,776), and hybrid promoters such as the tac promoter (deBoer et al., Proc. Natl. Acad. Sci. USA, 80:21-25 (1983)). Promoters for use in bacterial systems also will contain a Shine-Dalgarno (S.D.) sequence operably linked to the DNA encoding DR6 and/or APP polypeptide.

Examples of suitable promoting sequences for use with yeast hosts include the promoters for 3-phosphoglycerate kinase (Hitzeman et al., J. Biol. Chem., 255:2073 (1980)) or other glycolytic enzymes (Hess et al., J. Adv. Enzyme Reg., 7:149 (1968); Holland, Biochemistry, 17:4900 (1978)), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

DR6 and/or APP polypeptide transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the DR6 and/or APP polypeptide by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, alpha-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the DR6 and/or APP polypeptide coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding DR6 polypeptide.

Still other methods, vectors, and host cells suitable for adaptation to the synthesis of DR6 and/or APP polypeptide in recombinant vertebrate cell culture are described in Gething et al., Nature, 293:620-625 (1981); Mantei et al., Nature, 281:40-46 (1979); EP 117,060; and EP 117,058.

### Culturing the Host Cells

The host cells used to produce the DR6 and/or APP polypeptide of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. No. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### Detecting Gene Amplification/Expression

Gene amplification and/or expression may be measured in a sample directly, for example, by conventional Southern blotting, Northern blotting to quantitate the transcription of mRNA (Thomas, Proc. Natl. Acad. Sci. USA, 77:5201-5205 (1980)), dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal Conveniently, the antibodies may be prepared against a native sequence DR6 polypeptide or against a synthetic peptide based on the DR6 sequences provided herein or against exogenous sequence fused to DR6 DNA and encoding a specific antibody epitope.

### Purification of DR6 Polypeptide

Forms of DR6 and/or APP polypeptide may be recovered from culture medium or from host cell lysates. If membrane-bound, it can be released from the membrane using a suitable detergent solution (e.g. Triton-X 100) or by enzymatic cleavage. Cells employed in expression of DR6 polypeptide can be disrupted by various physical or chemical means, such as freeze-thaw cycling, sonication, mechanical disruption, or cell lysing agents.

It may be desired to purify DR6 and/or APP polypeptide from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the DR6 and/or APP polypeptide. Various methods of protein purification may be employed and such methods are known in the art and described for example in Deutscher, Methods in Enzymology, 182 (1990); Scopes, PROTEIN PURIFICATION: PRINCIPLES AND PRACTICE, Springer-Verlag, New York (1982). The purification step(s) selected will depend, for example, on the nature of the production process used and the particular DR6 polypeptide produced.

Soluble forms of DR6 and/or APP may be employed as DR6 antagonists in the methods of the invention. Such soluble forms of DR6 and/or APP may comprise modifications, as described below (such as by fusing to an immunoglobulin, epitope tag or leucine zipper). Immunoadhesin molecules are further contemplated for use in the methods herein. DR6 and/or APP immunoadhesins may comprise various forms of DR6 and/or APP, such as the full length polypeptide as well as soluble, extracellular domain forms of the DR6 and/or APP or a fragment thereof. In particular embodiments, the molecule may comprise a fusion of the DR6 polypeptide with an immunoglobulin or a particular region of an immunoglobulin. For a bivalent form of the immunoadhesin, such a fusion could be to the Fc region of an IgG molecule. The Ig fusions preferably include the substitution of a soluble (transmembrane domain deleted or inactivated) form of the polypeptide in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions, see also U.S. Pat. No. 5,428,130 issued Jun. 27, 1995 and Chamow et al., TIBTECH, 14:52-60 (1996).

An optional immunoadhesin design combines the binding domain(s) of the adhesin (e.g. a DR6 and/or APP ectodomain) with the Fc region of an immunoglobulin heavy chain. Ordinarily, when preparing the immunoadhesins of the present invention, nucleic acid encoding the binding domain of the adhesin will be fused C-terminally to nucleic acid encoding the N-terminus of an immunoglobulin constant domain sequence, however N-terminal fusions are also possible.

Typically, in such fusions the encoded chimeric polypeptide will retain at least functionally active hinge, C_{H}2 and C_{H}3 domains of the constant region of an immunoglobulin heavy chain. Fusions are also made to the C-terminus of the Fc portion of a constant domain, or immediately N-terminal to the C_{H}1 of the heavy chain or the corresponding region of the light chain. The precise site at which the fusion is made is not critical; particular sites are well known and may be selected in order to optimize the biological activity, secretion, or binding characteristics of the immunoadhesin.

In a preferred embodiment, the adhesin sequence is fused to the N-terminus of the Fc region of immunoglobulin G₁ (IgG₁). It is possible to fuse the entire heavy chain constant region to the adhesin sequence. However, more preferably, a sequence beginning in the hinge region just upstream of the papain cleavage site which defines IgG Fc chemically (i.e. residue 216, taking the first residue of heavy chain constant region to be 114), or analogous sites of other immunoglobulins is used in the fusion. In a particularly preferred embodiment, the adhesin amino acid sequence is fused to (a) the hinge region and C_{H}2 and C_{H}3 or (b) the C_{H}1, hinge, C_{H}2 and C_{H}3 domains, of an IgG heavy chain.

For bispecific immunoadhesins, the immunoadhesins are assembled as multimers, and particularly as heterodimers or heterotetramers. Generally, these assembled immunoglobulins will have known unit structures. A basic four chain structural unit is the form in which IgG, IgD, and IgE exist. A four chain unit is repeated in the higher molecular weight immunoglobulins; IgM generally exists as a pentamer of four basic units held together by disulfide bonds. IgA globulin, and occasionally IgG globulin, may also exist in multimeric form in serum. In the case of multimer, each of the four units may be the same or different.

Various exemplary assembled immunoadhesins within the scope herein are schematically diagrammed below:
(a) AC_{L}-AC_{L};
(b) AC_{H}-(AC_{H}, AC_{L}-AC_{H}, AC_{L}-V_{H}C_{H}, or V_{L}C_{L}-AC_{H});
(c) AC_{L}-AC_{H}-(AC_{L}-AC_{H}, AC_{L}-V_{H}C_{H}, V_{L}C_{L}-AC_{H}, or V_{L}C_{L}-V_{H}C_{H})
(d) AC_{L}-V_{H}C_{H}-(AC_{H}, or AC_{L}-V_{H}C_{H}, or V_{L}C_{L}-AC_{H});
(e) V_{L}C_{L}-AC_{H}-(AC_{L}-V_{H}C_{H}, or V_{L}C_{L}-AC_{H}); and
(f) (A-Y)ₙ-(V_{L}C_{L}-V_{H}C_{H})₂;
   wherein each A represents identical or different adhesin amino acid sequences;
V_{L} is an immunoglobulin light chain variable domain;
V_{H} is an immunoglobulin heavy chain variable domain;
C_{L} is an immunoglobulin light chain constant domain;
C_{H} is an immunoglobulin heavy chain constant domain;
n is an integer greater than 1;
Y designates the residue of a covalent cross-linking agent.

In the interests of brevity, the foregoing structures only show key features; they do not indicate joining (J) or other domains of the immunoglobulins, nor are disulfide bonds shown. However, where such domains are required for binding activity, they shall be constructed to be present in the ordinary locations which they occupy in the immunoglobulin molecules.

Alternatively, the adhesin sequences can be inserted between immunoglobulin heavy chain and light chain sequences, such that an immunoglobulin comprising a chimeric heavy chain is obtained. In this embodiment, the adhesin sequences are fused to the 3′ end of an immunoglobulin heavy chain in each arm of an immunoglobulin, either between the hinge and the C_{H}2 domain, or between the C_{H}2 and C_{H}3 domains. Similar constructs have been reported by Hoogenboom et al., Mol. Immunol., 28:1027-1037 (1991).

Although the presence of an immunoglobulin light chain is not required in the immunoadhesins of the present invention, an immunoglobulin light chain might be present either covalently associated to an adhesin-immunoglobulin heavy chain fusion polypeptide, or directly fused to the adhesin. In the former case, DNA encoding an immunoglobulin light chain is typically coexpressed with the DNA encoding the adhesin-immunoglobulin heavy chain fusion protein. Upon secretion, the hybrid heavy chain and the light chain will be covalently associated to provide an immunoglobulin-like structure comprising two disulfide-linked immunoglobulin heavy chain-light chain pairs. Methods suitable for the preparation of such structures are, for example, disclosed in U.S. Pat. No. 4,816,567, issued 28 Mar. 1989.

Immunoadhesins are most conveniently constructed by fusing the cDNA sequence encoding the adhesin portion in-frame to an immunoglobulin cDNA sequence. However, fusion to genomic immunoglobulin fragments can also be used (see, e.g. Aruffo et al., Cell, 61:1303-1313 (1990); and Stamenkovic et al., Cell, 66:1133-1144 (1991)). The latter type of fusion requires the presence of Ig regulatory sequences for expression. cDNAs encoding IgG heavy-chain constant regions can be isolated based on published sequences from cDNA libraries derived from spleen or peripheral blood lymphocytes, by hybridization or by polymerase chain reaction (PCR) techniques. The cDNAs encoding the “adhesin” and the immunoglobulin parts of the immunoadhesin are inserted in tandem into a plasmid vector that directs efficient expression in the chosen host cells.

In another embodiment, the DR6 antagonist may be covalently modified by linking the receptor polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337, or other like molecules such as polyglutamate. Such pegylated forms may be prepared using techniques known in the art.

Leucine zipper forms of these molecules are also contemplated by the invention. “Leucine zipper” is a term in the art used to refer to a leucine rich sequence that enhances, promotes, or drives dimerization or trimerization of its fusion partner (e.g., the sequence or molecule to which the leucine zipper is fused or linked to). Various leucine zipper polypeptides have been described in the art. See, e.g., Landschulz et al., Science, 240:1759 (1988); U.S. Pat. No. 5,716,805; WO 94/10308; Hoppe et al., FEBS Letters, 344:1991 (1994); Maniatis et al., Nature, 341:24 (1989). Those skilled in the art will appreciate that a leucine zipper sequence may be fused at either the 5′ or 3′ end of the DR6 molecule.

The DR6 and/or APP polypeptides of the present invention may also be modified in a way to form chimeric molecules by fusing the polypeptide to another, heterologous polypeptide or amino acid sequence. Preferably, such heterologous polypeptide or amino acid sequence is one which acts to oligimerize the chimeric molecule. In one embodiment, such a chimeric molecule comprises a fusion of the DR6 and/or APP polypeptide with a tag polypeptide which provides an epitope to which an anti-tag antibody can selectively bind. The epitope tag is generally placed at the amino- or carboxyl-terminus of the polypeptide. The presence of such epitope-tagged forms of the polypeptide can be detected using an antibody against the tag polypeptide. Also, provision of the epitope tag enables the polypeptide to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 (Field et al., Mol. Cell. Biol., 8:2159-2165 (1988)); the c-myc tag and the 8F9, 3C7, 6E10, G4, B7 and 9E10 antibodies thereto (Evan et al., Mol. Cell. Biol., 5:3610-3616 (1985)); and the Herpes Simplex virus glycoprotein D (gD) tag and its antibody (Paborsky et al., Protein Engineering, 3(6):547-553 (1990)). Other tag polypeptides include the Flag-peptide (Hopp et al., BioTechnology, 6:1204-1210 (1988)); the KT3 epitope peptide (Martin et al., Science, 255:192-194 (1992)); an alpha-tubulin epitope peptide (Skinner et al., J. Biol. Chem., 266:15163-15166 (1991)); and the T7 gene 10 protein peptide tag (Lutz-Freyermuth et al., Proc. Natl. Acad. Sci. USA, 87:6393-6397 (1990)).

### Anti-DR6 and Anti-APP Antibodies

In other embodiments of the invention, DR6 and/or APP antibodies are provided. Exemplary antibodies include polyclonal, monoclonal, humanized, bispecific, and heteroconjugate antibodies. These anti-DR6 and/or APP antibodies are preferably DR6 antagonist antibodies.

### Polyclonal Antibodies

The antibodies of the invention may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include DR6 and/or APP polypeptide (e.g. a DR6 and/or APP ECD) or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation. The mammal can then be bled, and the serum assayed for DR6 and/or APP antibody titer. If desired, the mammal can be boosted until the antibody titer increases or plateaus.

### Monoclonal Antibodies

The antibodies of the invention may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the DR6 and/or APP polypeptide (e.g. a DR6 and/or APP ECD) or a fusion protein thereof, such as a DR6 ECD-IgG and/or APP sAPP-IgG fusion protein.

Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. An example of such a murine myeloma cell line is P3X63Ag8U.1, (ATCC CRL 1580). Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against DR6 and/or APP. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980) or by way of BiaCore analysis.

After the desired hybridoma cells are identified, the clones may be subcloned by limiting dilution procedures and grown by standard methods (Goding, supra). Suitable culture media for this purpose include, for example, Dulbecco's Modified Eagle's Medium or RPMI-1640 medium. Alternatively, the hybridoma cells may be grown in vivo as ascites in a mammal

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences, Morrison, et al., Proc. Nat. Acad. Sci. USA 81, 6851 (1984), or by covalently joining to the immunoglobulin coding sequence all or part of the coding sequence for a non-immunoglobulin polypeptide. In that manner, “chimeric” or “hybrid” antibodies are prepared that have the binding specificity of an anti-DR6 monoclonal antibody herein.

Typically such non-immunoglobulin polypeptides are substituted for the constant domains of an antibody of the invention, or they are substituted for the variable domains of one antigen-combining site of an antibody of the invention to create a chimeric bivalent antibody comprising one antigen-combining site having specificity for DR6 and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate.

Single chain Fv fragments may also be produced, such as described in Iliades et al., FEBS Letters, 409:437-441 (1997). Coupling of such single chain fragments using various linkers is described in Kortt et al., Protein Engineering, 10:423-433 (1997). A variety of techniques for the recombinant production and manipulation of antibodies are well known in the art. Illustrative examples of such techniques that are typically utilized by skilled artisans are described in greater detail below.

### Humanized Antibodies

Generally, a humanized antibody has one or more amino acid residues introduced into it from a non-human source. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody.

Accordingly, such “humanized” antibodies are chimeric antibodies wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

It is important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e. the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding.

### Human Antibodies

Human monoclonal antibodies can be made by the hybridoma method. Human myeloma and mouse-human heteromyeloma cell lines for the production of human monoclonal antibodies have been described, for example, by Kozbor, J. Immunol. 133, 3001 (1984), and Brodeur, et al., MONOCLONAL ANTIBODY PRODUCTION TECHNIQUES AND APPLICATIONS, pp. 51-63 (Marcel Dekker, Inc., New York, 1987).

It is now possible to produce transgenic animals (e.g. mice) that are capable, upon immunization, of producing a repertoire of human antibodies in the absence of endogenous immunoglobulin production. For example, it has been described that the homozygous deletion of the antibody heavy chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g. Jakobovits et al., Proc. Natl. Acad. Sci. USA 90, 2551-255 (1993); Jakobovits et al., Nature 362, 255-258 (1993).

Mendez et al. (Nature Genetics 15: 146-156 (1997)) have further improved the technology and have generated a line of transgenic mice designated as “Xenomouse II” that, when challenged with an antigen, generates high affinity fully human antibodies. This was achieved by germ-line integration of megabase human heavy chain and light chain loci into mice with deletion into endogenous J_{H} segment as described above. The Xenomouse II harbors 1,020 kb of human heavy chain locus containing approximately 66 V_{H} genes, complete D_{H} and J_{H} regions and three different constant regions (μ, δ and χ), and also harbors 800 kb of human κ locus containing 32 Vκ genes, Jκ segments and Cκ genes. The antibodies produced in these mice closely resemble that seen in humans in all respects, including gene rearrangement, assembly, and repertoire. The human antibodies are preferentially expressed over endogenous antibodies due to deletion in endogenous J_{H} segment that prevents gene rearrangement in the murine locus.

Alternatively, the phage display technology (McCafferty et al., Nature 348, 552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g. Johnson, Kevin S. and Chiswell, David J., Current Opinion in Structural Biology 3, 564-571 (1993). Several sources of V-gene segments can be used for phage display. Clackson et al., Nature 352, 624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222, 581-597 (1991), or Griffith et al., EMBO J. 12, 725-734 (1993). In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling” (Marks et al., Bio/Technol. 10, 779-783 [1992]). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the nM range. A strategy for making very large phage antibody repertoires (also known as “the mother-of-all libraries”) has been described by Waterhouse et al., Nucl. Acids Res. 21, 2265-2266 (1993). Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable capable of restoring a functional antigen-binding site, i.e. the epitope governs (imprints) the choice of partner. When the process is repeated in order to replace the remaining rodent V domain, a human antibody is obtained (see PCT patent application WO 93/06213, published 1 Apr. 1993). Unlike traditional humanization of rodent antibodies by CDR grafting, this technique provides completely human antibodies, which have no framework or CDR residues of rodent origin.

As discussed in detail below, the antibodies of the invention may optionally comprise monomeric, antibodies, dimeric antibodies, as well as multivalent forms of antibodies. Those skilled in the art may construct such dimers or multivalent forms by techniques known in the art and using the DR6 and/or APP antibodies herein. Methods for preparing monovalent antibodies are also well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

### Bispecific Antibodies

Bispecific antibodies are monoclonal, preferably human or humanized, antibodies that have binding specificities for at least two different antigens. In the present case, one of the binding specificities is for the DR6 receptor, the other one is for any other antigen, and preferably for another receptor or receptor subunit. Methods for making bispecific antibodies are known in the art. Traditionally, the recombinant production of bispecific antibodies is based on the coexpression of two immunoglobulin heavy chain-light chain pairs, where the two heavy chains have different specificities (Millstein and Cuello, Nature 305, 537-539 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed in PCT application publication No. WO 93/08829 (published 13 May 1993), and in Traunecker et al., EMBO J. 10, 3655-3659 (1991).

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2 and CH3 regions. It is preferred to have the first heavy chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance. In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in PCT Publication No. WO 94/04690, published on Mar. 3, 1994.

For further details of generating bispecific antibodies see, for example, Suresh et al., Meth. Enzymol. 121, 210 (1986).

### Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (PCT application publication Nos. WO 91/00360 and WO 92/200373; EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

### Antibody Fragments

In certain embodiments, the anti-DR6 and/or APP antibody (including murine, human and humanized antibodies, and antibody variants) is an antibody fragment. Various techniques have been developed for the production of antibody fragments. Traditionally, these fragments were derived via proteolytic digestion of intact antibodies (see, e.g., Morimoto et al., J. Biochem. Biophys. Methods 24:107-117 (1992) and Brennan et al., Science 229:81 (1985)). However, these fragments can now be produced directly by recombinant host cells. For example, Fab′-SH fragments can be directly recovered from E. coli and chemically coupled to form F(ab′)₂ fragments (Carter et al., Bio/Technology 10:163-167 (1992)). In another embodiment, the F(ab′)₂ is formed using the leucine zipper GCN4 to promote assembly of the F(ab′)₂ molecule. According to another approach, Fv, Fab or F(ab′)₂ fragments can be isolated directly from recombinant host cell culture. A variety of techniques for the production of antibody fragments will be apparent to the skilled practitioner. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields an F(ab′)₂ fragment that has two antigen combining sites and is still capable of cross-linking antigen.

The Fab fragments produced in the antibody digestion also contain the constant domains of the light chain and the first constant domain (CHO of the heavy chain. Fab′ fragments differ from Fab fragments by the addition of a few residues at the carboxy terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

### Glycosylation Variants of Antibodies

Antibodies are glycosylated at conserved positions in their constant regions (Jefferis and Lund, Chem. Immunol. 65:111-128 (1997); Wright and Morrison, TibTECH 15:26-32 [1997]). The oligosaccharide side chains of the immunoglobulins affect the protein's function (Boyd et al., Mol. Immunol. 32:1311-1318 (1996); Wittwe and Howard, Biochem. 29:4175-4180 (1990)), and the intramolecular interaction between portions of the glycoprotein which can affect the conformation and presented three-dimensional surface of the glycoprotein (Hefferis and Lund, supra; Wyss and Wagner, Current Opin. Biotech. 7:409-416 (1996)). Oligosaccharides may also serve to target a given glycoprotein to certain molecules based upon specific recognition structures. For example, it has been reported that in agalactosylated IgG, the oligosaccharide moiety ‘flips’ out of the inter-CH2 space and terminal N-acetylglucosamine residues become available to bind mannose binding protein (Malhotra et al., Nature Med. 1:237-243 (1995)). Removal by glycopeptidase of the oligosaccharides from CAMPATH-1H (a recombinant humanized murine monoclonal IgG1 antibody which recognizes the CDw52 antigen of human lymphocytes) produced in Chinese Hamster Ovary (CHO) cells resulted in a complete reduction in complement mediated lysis (CMCL) (Boyd et al., Mol. Immunol. 32:1311-1318 [1996]), while selective removal of sialic acid residues using neuraminidase resulted in no loss of DMCL. Glycosylation of antibodies has also been reported to affect antibody-dependent cellular cytotoxicity (ADCC). In particular, CHO cells with tetracycline-regulated expression β(1,4)-N-acetylglucosaminyltransferase III (GnTIII), a glycosyltransferase catalyzing formation of bisecting GlcNAc, was reported to have improved ADCC activity (Umana et al., Mature Biotech. 17:176-180 (1999)).

Glycosylation variants of antibodies are variants in which the glycosylation pattern of an antibody is altered. By altering is meant deleting one or more carbohydrate moieties found in the antibody, adding one or more carbohydrate moieties to the antibody, changing the composition of glycosylation (glycosylation pattern), the extent of glycosylation, etc. Glycosylation variants may, for example, be prepared by removing, changing and/or adding one or more glycosylation sites in the nucleic acid sequence encoding the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

The glycosylation (including glycosylation pattern) of antibodies may also be altered without altering the underlying nucleotide sequence. Glycosylation largely depends on the host cell used to express the antibody. Since the cell type used for expression of recombinant glycoproteins, e.g. antibodies, as potential therapeutics is rarely the native cell, significant variations in the glycosylation pattern of the antibodies can be expected (see, e.g. Hse et al., J. Biol. Chem. 272:9062-9070 (1997)). In addition to the choice of host cells, factors which affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261 and 5,278,299). Glycosylation, or certain types of glycosylation, can be enzymatically removed from the glycoprotein, for example using endoglycosidase H (Endo H). In addition, the recombinant host cell can be genetically engineered, e.g. make defective in processing certain types of polysaccharides. These and similar techniques are well known in the art.

The glycosylation structure of antibodies can be readily analyzed by conventional techniques of carbohydrate analysis, including lectin chromatography, NMR, Mass spectrometry, HPLC, GPC, monosaccharide compositional analysis, sequential enzymatic digestion, and HPAEC-PAD, which uses high pH anion exchange chromatography to separate oligosaccharides based on charge. Methods for releasing oligosaccharides for analytical purposes are also known, and include, without limitation, enzymatic treatment (commonly performed using peptide-N-glycosidase F/endo-β-galactosidase), elimination using harsh alkaline environment to release mainly O-linked structures, and chemical methods using anhydrous hydrazine to release both N- and O-linked oligosaccharides.

### Exemplary Antibodies

As described in the Examples below, anti-DR6 monoclonal antibodies have been identified. In optional embodiments, the DR6 antibodies of the invention will have the same biological characteristics as any of the anti-DR6 and/or APP antibodies specifically disclosed herein.

The term “biological characteristics” is used to refer to the in vitro and/or in vivo activities or properties of the monoclonal antibody, such as the ability to specifically bind to DR6 or to block, inhibit, or neutralize DR6 activation. The properties and activities of the DR6 and/or APP antibodies are further described in the Examples below.

Optionally, the monoclonal antibodies of the present invention will have the same biological characteristics as any of the antibodies specifically characterized in the Examples below, and/or bind to the same epitope(s) as these antibodies. This can be determined by conducting various assays, such as described herein and in the Examples. For instance, to determine whether a monoclonal antibody has the same specificity as the DR6 and/or APP antibodies specifically referred to herein, one can compare its activity in competitive binding assays. In addition, an epitope to which a particular anti-DR6 and/or APP antibody binds can be determined by crystallography study of the complex between DR6 and/or APP and the antibody in question.

The DR6 and/or APP antibodies, as described herein, will preferably possess the desired DR6 antagonistic activity. Such DR6 antibodies may include but are not limited to chimeric, humanized, human, and affinity matured antibodies. As described above, the DR6 and/or APP antibodies may be constructed or engineered using various techniques to achieve these desired activities or properties.

Additional embodiments of the invention include an anti-DR6 receptor and/or APP ligand antibody disclosed herein which is linked to one or more non-proteinaceous polymers selected from the group consisting of polyethylene glycol, polypropylene glycol, and polyoxyalkylene. Optionally, an anti-DR6 receptor and/or APP ligand antibody disclosed herein is glycosylated or alternatively, unglycosylated.

The antibodies of the invention include “cross-linked” DR6 and/or APP antibodies. The term “cross-linked” as used herein refers to binding of at least two IgG molecules together to form one (or single) molecule. The DR6 and/or APP antibodies may be cross-linked using various linker molecules, preferably the DR6 and/or APP antibodies are cross-linked using an anti-IgG molecule, complement, chemical modification or molecular engineering. It is appreciated by those skilled in the art that complement has a relatively high affinity to antibody molecules once the antibodies bind to cell surface membrane. Accordingly, it is believed that complement may be used as a cross-linking molecule to link two or more anti-DR6 antibodies bound to cell surface membrane.

The invention also provides isolated nucleic acids encoding DR6 and/or APP antibodies as disclosed herein, vectors and host cells comprising the nucleic acid, and recombinant techniques for the production of the antibody.

For recombinant production of the antibody, the nucleic acid encoding it is isolated and inserted into a replicable vector for further cloning (amplification of the DNA) or for expression. DNA encoding the antibody is readily isolated and sequenced using conventional procedures (e.g., by using oligonucleotide probes that are capable of binding specifically to genes encoding the antibody). Many vectors are available. The vector components generally include, but are not limited to, one or more of the following: a signal sequence, an origin of replication, one or more marker genes, an enhancer element, a promoter, and a transcription termination sequence.

The methods herein include methods for the production of chimeric or recombinant anti-DR6 and/or APP antibodies which comprise the steps of providing a vector comprising a DNA sequence encoding an anti-DR6 and/or APP antibody light chain or heavy chain (or both a light chain and a heavy chain), transfecting or transforming a host cell with the vector, and culturing the host cell(s) under conditions sufficient to produce the recombinant anti-DR6 antibody and/or APP antibody product.

### Formulations of DR6 Antagonists

In the preparation of typical formulations herein, it is noted that the recommended quality or “grade” of the components employed will depend on the ultimate use of the formulation. For therapeutic uses, it is preferred that the component(s) are of an allowable grade (such as “GRAS”) as an additive to pharmaceutical products.

In certain embodiments, there are provided compositions comprising DR6 antagonist(s) and one or more excipients which provide sufficient ionic strength to enhance solubility and/or stability of the DR6 antagonist, wherein the composition has a pH of 6 (or about 6) to 9 (or about 9). The DR6 antagonist may be prepared by any suitable method to achieve the desired purity of the protein, for example, according to the above methods. In certain embodiments, the DR6 antagonist is recombinantly expressed in host cells or prepared by chemical synthesis. The concentration of the DR6 antagonist in the formulation may vary depending, for instance, on the intended use of the formulation. Those skilled in the art can determine without undue experimentation the desired concentration of the DR6 antagonist.

The one or more excipients in the formulations which provide sufficient ionic strength to enhance solubility and/or stability of the DR6 antagonist is optionally a polyionic organic or inorganic acid, aspartate, sodium sulfate, sodium succinate, sodium acetate, sodium chloride, Captisol™, Tris, arginine salt or other amino acids, sugars and polyols such as trehalose and sucrose. Preferably the one or more excipients in the formulations which provide sufficient ionic strength is a salt. Salts which may be employed include but are not limited to sodium salts and arginine salts. The type of salt employed and the concentration of the salt are preferably such that the formulation has a relatively high ionic strength which allows the DR6 antagonist in the formulation to be stable. Optionally, the salt is present in the formulation at a concentration of about 20 mM to about 0.5 M.

The composition preferably has a pH of 6 (or about 6) to 9 (or about 9), more preferably about 6.5 to about 8.5, and even more preferably about 7 to about 7.5. In a preferred aspect of this embodiment, the composition will further comprise a buffer to maintain the pH of the composition at least about 6 to about 8. Examples of buffers which may be employed include but are not limited to Tris, HEPES, and histidine. When employing Tris, the pH may optionally be adjusted to about 7 to 8.5. When employing Hepes or histidine, the pH may optionally be adjusted to about 6.5 to 7. Optionally, the buffer is employed at a concentration of about 5 mM to about 50 mM in the formulation.

Particularly for liquid formulations (or reconstituted lyophilized formulations), it may be desirable to include one or more surfactants in the composition. Such surfactants may, for instance, comprise a non-ionic surfactant like TWEEN™ or PLURONICS™ (e.g., polysorbate or poloxamer). Preferably, the surfactant comprises polysorbate 20 (“Tween 20”). The surfactant will optionally be employed at a concentration of about 0.005% to about 0.2%.

The formulations of the present invention may include, in addition to DR6 antagonist(s) and those components described above, further various other excipients or components. Optionally, the formulation may contain, for parenteral administration, a pharmaceutically or parenterally acceptable carrier, i.e., one that is non-toxic to recipients at the dosages and concentrations employed and is compatible with other ingredients of the formulation. Optionally, the carrier is a parenteral carrier, such as a solution that is isotonic with the blood of the recipient. Examples of such carrier vehicles include water, saline or a buffered solution such as phosphate-buffered saline (PBS), Ringer's solution, and dextrose solution. Various optional pharmaceutically acceptable carriers, excipients, or stabilizers are described further in Remington's Pharmaceutical Sciences, 16th edition, Osol, A. ed. (1980).

The formulations herein also may contain one or more preservatives. Examples include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols, alkyl parabens such as methyl or propyl paraben, and m-cresol. Antioxidants include ascorbic acid and methionine; preservatives (such as octadecyldimethylbenzyl ammonium chloride; hexamethonium chloride; benzalkonium chloride, benzethonium chloride; butyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; or polyethylene glycol (PEG)

The compositions of the invention may comprise liquid formulations (liquid solutions or liquid suspensions), and lyophilized formulations, as well as suspension formulations.

The final formulation, if a liquid, is preferably stored frozen at <20° C. Alternatively, the formulation can be lyophilized and provided as a powder for reconstitution with water for injection that optionally may be stored at 2-30° C.

The formulation to be used for therapeutic administration must be sterile. Sterility is readily accomplished by filtration through sterile filtration membranes (e.g., 0.2 micron membranes). Therapeutic compositions generally are placed into a container having a sterile access port, for example, an intravenous solution bag or vial having a stopper pierceable by a hypodermic injection needle.

The composition ordinarily will be stored in single unit or multi-dose containers, for example, sealed ampules or vials, as an aqueous solution or as a lyophilized formulation for reconstitution. The containers may any available containers in the art and filled using conventional methods. Optionally, the formulation may be included in an injection pen device (or a cartridge which fits into a pen device), such as those available in the art (see, e.g., U.S. Pat. No. 5,370,629), which are suitable for therapeutic delivery of the formulation. An injection solution can be prepared by reconstituting the lyophilized DR6 antagonist formulation using, for example, Water-for-Injection.

### Therapies Using DR6 Antagonist(s)

The DR6 antagonists of the invention have various utilities. DR6 antagonists are useful in the diagnosis and treatment of neurological disorders. Diagnosis in mammals of the various pathological conditions described herein can be made by the skilled practitioner. Diagnostic techniques are available in the art which allow, e.g., for the diagnosis or detection of various neurological disorders in a mammal

Neurological disorders contemplated for treatment by the present invention include familial and sporadic amyotrophic lateral sclerosis (FALS and ALS, respectively), familial and sporadic Parkinson's disease, Huntington's disease, familial and sporadic Alzheimer's disease and Spinal Muscular Atrophy (SMA) (Price et al., supra). Many of these diseases are typified by onset during the middle adult years and lead to rapid degeneration of specific subsets of neurons within the neural system, ultimately resulting in premature death.

Amyotrophic lateral sclerosis (ALS) is the most commonly diagnosed progressive motor neuron disease. The disease is characterized by degeneration of motor neurons in the cortex, brainstem and spinal cord (Siddique et al., J. Neural Transm. Suppl., 49:219-233 (1997); Siddique et al., Neurology, 47: (4 Suppl 2):S27-34; discussion S34-5 (1996); Rosen et al., Nature, 362:59-62 (1993); Gurney et al., Science, 264:1772-1775 1994)).

Parkinson's disease (paralysis agitans) is a common neurodegenerative disorder which usually appears in mid to late life. Familial and sporadic cases occur, although familial cases account for only 1-2 percent of the observed cases. Patients frequently have nerve cell loss with reactive gliosis and Lewy bodies in the substantia nigra and locus coeruleus of the brain stem. As a class, the nigrostriatal dopaminergic neurons seem to be most affected (Uhl et al., Neurology, 35:1215-1218 (1985); Levine et al., Trends Neurosci., 27:691-697 (2004); Fleming et al., NeuroRx, 2:495-503 (2005)).

Proximal spinal muscular atrophy (SMA) is a common autosomal recessive neurodegenerative disease in humans typically characterized by loss of the spinal motor neurons and atrophy of the limb and trunk muscles (Monani et al., Hum. Mol. Genet., 9:2451-2457 (2000); Monani et al., J. Cell Biol., 160:41-52 (2003)). It occurs with a frequency of 1 in 10,000 individuals and is the most common genetic cause of infant mortality. Based on the age at onset and severity of the disease phenotype, the proximal SMAs have been classified into type I (severe), type II (intermediate), and type III (mild) SMA. All three forms of the disease are due to loss or mutation of the telomeric survival of motor neurons gene (SMN1) (Monani et al., supra, 2000; Monani et al., supra, 2003)).

Neuronal cell loss has been reported in a number of neurodegenerative diseases, including Alzheimer's disease, Parkinson's disease, Amyotrophic Lateral Sclerosis (ALS), and Spinal Muscular Atrophy (SMA).

Optionally, diagnosis of Alzheimer's disease in a patient may be based on the criteria of the Diagnostic and Statistical Manual of Mental disorders, 4th Edition (DSM-IV-TR) (see, e.g. American Psychiatric Association. Diagnostic and statistical manual of mental disorders, 4th Edition—text revised. Washington, D.C.: 2000). Briefly, the DSM-IV-TR criteria include: (A) the development of multiple cognitive deficits manifested by both memory impairment and one or more of the following: (1) aphasia; (2) apraxia; (3) agnosia; or (4) disturbances in executive functioning; (B) the cognitive deficits represent a decline from previous functioning and cause significant impairment in social or occupational functioning; (C) the course is characterized by gradual onset and continuing decline; (D) the cognitive deficits are not due to other central nervous system, systemic, or substance-induced conditions that cause progressive deficits in memory and cognition; and (E) the disturbance is not better accounted for by another psychiatric disorder. Alternative criteria by which diagnosis of Alzheimer's disease may be made include those based on the National Institute of Neurological and Communicative Disorders and Stroke-Alzheimer's Disease and Related Disorder Association (NINDS-ADRDA) working group criteria for Alzheimer's disease (see, e.g. McKhann et al., Neurology 1984; 34: 939-944). Briefly, the NINCDS-ADRDA criteria for possible Alzheimer's disease includes a dementia syndrome with an atypical onset, presentation, or progression and without a known etiology where any co-morbid diseases capable of producing dementia are not believed to be the cause. The NINCDS-ADRDA criteria for probable Alzheimer's disease includes dementia established by clinical and neuropsychological examination and involves (a) progressive deficits in two or more areas of cognition, including memory; (b) onset between the ages of 40 and 90 years; and (c) absence of systemic or other brain diseases capable of producing a dementia syndrome, including delirium. The NINCDS-ADRDA criteria for definite Alzheimer's disease includes meeting the criteria for probable Alzheimer's disease and has histopathologic evidence of Alzheimer's disease via autopsy or biopsy.

Revised NINDS-ADRDA diagnostic criteria have been proposed in Dubois et al., The Lancet Neurology, Volume 6, Issue 8, August 2007, Pages 734-746. As outlined briefly below, to meet this criteria for probable Alzheimer's disease, an affected individual must fulfill criterion A (the core clinical criterion) and at least one or more of the supportive biomarker criteria noted in B, C, D, or E. In this context, criterion A is characterized by the presence of an early and significant episodic memory impairment that includes the following features: (1) gradual and progressive change in memory function reported by patients or informants over more than 6 months; (2) objective evidence of significantly impaired episodic memory on testing: this generally consists of recall deficit that does not improve significantly or does not normalize with cueing or recognition testing and after effective encoding of information has been previously controlled; (3) the episodic memory impairment can be isolated or associated with other cognitive changes at the onset of AD or as AD advances. Criterion B is characterized by the presence of medial temporal lobe atrophy, as shown for example by: volume loss of hippocampi, entorhinal cortex, amygdala evidenced on MRI with qualitative ratings using visual scoring (referenced to well characterized population with age norms) or quantitative volumetry of regions of interest (referenced to well characterized population with age norms). Criterion C is characterized by an abnormal cerebrospinal fluid biomarker, for example low amyloid β₁₋₄₂ concentrations, increased total tau concentrations, or increased phospho-tau concentrations, or combinations of the three. Criterion C is characterized by a specific pattern on functional neuroimaging with PET, for example reduced glucose metabolism in bilateral temporal parietal regions. Criterion E is characterized by proven AD autosomal dominant mutation within the immediate family. AD is considered definite if the following are present: (1) both clinical and histopathological (brain biopsy or autopsy) evidence of the disease, as required by the NIA-Reagan criteria for the post-mortem diagnosis of AD; criteria must be present (see, e.g. Neurobiol Aging 1997; 18: S1-S2); and (2) both clinical and genetic evidence (mutation on chromosome 1, 14, or 21) of AD; criteria must be present.

In the methods of the invention, the DR6 antagonist is preferably administered to the mammal in a carrier; preferably a pharmaceutically-acceptable carrier. Suitable carriers and their formulations are described in REMINGTON's PHARMACEUTICAL SCIENCES, 16th ed., 1980, Mack Publishing Co., edited by Osol et al. Typically, an appropriate amount of a pharmaceutically-acceptable salt is used in the formulation to render the formulation isotonic. Examples of the carrier include saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of DR6 antagonist being administered.

The DR6 antagonist can be administered to the mammal by injection (e.g., intravenous, intraperitoneal, subcutaneous, intramuscular, intraportal), orally, or by other methods such as infusion that ensure its delivery to the bloodstream in an effective form. The DR6 antagonist may also be administered by isolated perfusion techniques, such as isolated tissue perfusion, or by intrathecal, intraoccularly, or lumbar puncture to exert local therapeutic effects. DR6 antagonists that do not readily cross the blood-brain barrier may be given directly, e.g., intracerebrally or into the spinal cord space or otherwise, that will transport them across the barrier. Effective dosages and schedules for administering the DR6 antagonist may be determined empirically, and making such determinations is within the skill in the art. Those skilled in the art will understand that the dosage of DR6 antagonist that must be administered will vary depending on, for example, the mammal which will receive the antagonist, the route of administration, the particular type of antagonist used and other drugs being administered to the mammal Guidance in selecting appropriate doses is found in the literature, for example, on therapeutic uses of antibodies, e.g., HANDBOOK OF MONOCLONAL ANTIBODIES, Ferrone et al., eds., Noges Publications, Park Ridge, N.J., (1985) ch. 22 and pp. 303-357; Smith et al., ANTIBODIES IN HUMAN DIAGNOSIS AND THERAPY, Haber et al., eds., Raven Press, New York (1977) pp. 365-389. A typical daily dosage of DR6 antibody used alone might range from about 1 ng/kg to up to 100 mg/kg of body weight or more per day, depending on the factors mentioned above.

The DR6 antagonist may also be administered to the mammal in combination with one or more other therapeutic agents. Examples of such other therapeutic agents include epidermal growth factor receptor (EGFR) inhibitors, e.g., compounds that bind to or otherwise interact directly with EGFR and prevent or reduce its signalling activity, such as Tarceva, antibodies like C225, also referred to as cetuximab and Erbitux® (ImClone Systems Inc.), fully human ABX-EGF (panitumumab, Abgenix Inc.), as well as fully human antibodies known as E1.1, E2.4, E2.5, E6.2, E6.4, E2.11, E6. 3 and E7.6. 3 and described in U.S. Pat. No. 6,235,883; MDX-447 (Medarex Inc), as well as EGFR small molecule inhibitors such as compounds described in U.S. Pat. No. 5,616,582, U.S. Pat. No. 5,457,105, U.S. Pat. No. 5,475,001, U.S. Pat. No. 5,654,307, U.S. Pat. No. 5,679,683, U.S. Pat. No. 6,084,095, U.S. Pat. No. 6,265,410, U.S. Pat. No. 6,455,534, U.S. Pat. No. 6,521,620, U.S. Pat. No. 6,596,726, U.S. Pat. No. 6,713,484, U.S. Pat. No. 5,770,599, U.S. Pat. No. 6,140,332, U.S. Pat. No. 5,866,572, U.S. Pat. No. 6,399,602, U.S. Pat. No. 6,344,459, U.S. Pat. No. 6,602,863, U.S. Pat. No. 6,391,874, WO9814451, WO9850038, WO9909016, WO9924037, U.S. Pat. No. 6,344,455, U.S. Pat. No. 5,760,041, U.S. Pat. No. 6,002,008, U.S. Pat. No. 5,747,498; particular small molecule EGFR inhibitors include OSI-774 (CP-358774, erlotinib, OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); Iressa (ZD1839, gefitinib, 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N-8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); and EKB-569 (N-[4-[(3-chloro-4-fluorophenyl)amino]-3-cyano-7-ethoxy-6-quinolinyl]-4-(dimethylamino)-2-butenamide). Other therapeutic agents that may be employed include apoptosis inhibitors, particularly intracellular apoptosis inhibitors, e.g. caspase inhibitors such as caspase-3, caspase-6, or caspase-8 inhibitors, Bid inhibitors, Bax inhibitors or any combination thereof. Examples of suitable inhibitors are caspase inhibitors in general, dipeptide inhibitors, carbamate inhibitors, substituted aspartic acid acetals, heterocyclyldicarbamides, quinoline-(di-, tri-, tetrapeptide) derivatives, substituted 2-aminobenzamide caspase inhibitors, substituted a-hydroxy acid caspase inhibitors, inhibition by nitrosylation; CASP-1; CASP-3: protein-inhibitors, antisense molecules, nicotinyl-aspartyl-ketones, y-ketoacid dipeptide derivatives, CASP-8: antisense molecules, interacting proteins CASP-9, CASP2: antisense molecules; CASP-6: antisense molecules; CASP-7: antisense molecules; and CASP-12 inhibitors. Further examples are mitochondrial inhibitors such as Bcl-2-modulating factor; Bcl-2 mutant peptides derived from Bad, Bad, BH3-interacting domain death agonist, Bax inhibitor proteins and BLK genes and gene products. Further suitable intracellular modulators of apoptosis are modulators of CASP9/Apaf-1 association, antisense modulators of Apaf-1 expression, peptides for inhibition of apoptosis, anti-apoptotic compositions comprising the R1 subunit of Herpes Simplex virus, MEKK1 and fragments thereof, modulators of Survivin, modulators of inhibitors of apoptosis and HIAP2. Further examples of such agents include Minocycline (Neuroapoptosis Laboratory which inhibits cytochrome c release from mitochondria and blocks caspase-3 mRNA upregulation, Pifithrin alpha (UIC) which is a p53 inhibitor, CEP-1346 (Cephalon Inc.) which is a JNK pathway inhibitor, TCH346 (Novartis) which inhibits pro-apoptotic GAPDH signaling, IDN6556 (Idun Pharmaceuticals) which is a pan-caspase inhibitor; AZQs (AstraZeneca) which is a caspase-3 inhibitor, HMR-3480 (Aventis Pharma) which is a caspase-1/-4 inhibitor, and Activase/TPA (Genentech) which dissolves blood clots (thrombolytic drug).

Further suitable agents which may be administered, in addition to DR6 antagonist, include cholinesterase inhibitors (such as Donepezil, Galantamine, Rivastigmine, Tacrine), NMDA receptor antagonists (such as Memantine), Aβ aggregation inhibitors, antioxidants, γ-secretase modulators, NGF mimics or NGF gene therapy, PPARγ agonists, HMG-CoA reductase inhibitors (statins), ampakines, calcium channel blockers, GABA receptor antagonists, glycogen synthase kinase inhibitors, intravenous immunoglobulin, muscarinic receptor agonists, nicotinic receptor modulators, active or passive Aβ immunization, phosphodiesterase inhibitors, serotonin receptor antagonists and anti-Aβ antibodies (see, eg., WO 2007/062852; WO 2007/064972; WO 2003/040183; WO 1999/06066; WO 2006/081171; WO 1993/21526; EP 0276723B1; WO 2005/028511; WO 2005/082939).

The DR6 antagonist may be administered sequentially or concurrently with the one or more other therapeutic agents. The amounts of DR6 antagonist and therapeutic agent depend, for example, on what type of drugs are used, the pathological condition being treated, and the scheduling and routes of administration but would generally be less than if each were used individually.

Following administration of DR6 antagonist to the mammal, the mammal's physiological condition can be monitored in various ways well known to the skilled practitioner.

The therapeutic effects of the DR6 antagonists of the invention can be examined in in vitro assays and using in vivo animal models. A variety of well known assays and animal models can be used to test the efficacy of the candidate therapeutic agents. The in vivo nature of such models makes them particularly predictive of responses in human patients. Animal models of various neurodegenerative conditions and associated techniques for examining the pathological processes associated with these models of neurodegeneration (e.g. in the presence and absence of DR6 antagonists) are discussed in Example 14 below.

Animal models of various neurological disorders include both non-recombinant and recombinant (transgenic) animals. Non-recombinant animal models include, for example, rodent, e.g., murine models. Such models can be generated by introducing cells into syngeneic mice using standard techniques, e.g. subcutaneous injection, tail vein injection, spleen implantation, intraperitoneal implantation, and implantation under the renal capsule. In vivo models include models of stroke/cerebral ischemia, in vivo models of neurodegenerative diseases, such as mouse models of Parkinson's disease; mouse models of Alzheimer's disease; mouse models of amyotrophic lateral sclerosis ALS; mouse models of spinal muscular atrophy SMA; mouse/rat models of focal and global cerebral ischemia, for instance, common carotid artery occlusion model or middle cerebral artery occlusion models; or in ex vivo whole embryo cultures. The various assays may be conducted in known in vitro or in vivo assay formats, such as described below or as known in the art and described in the literature (See, e.g., McGowan et al., Trends in Genetics, 22:281-289 (2006); Fleming et al., NeuroRx, 2:495-503 (2005); Wong et al., Nature Neuroscience, 5:633-639 (2002)). Various such animal models are also available from commercial vendors such as the Jackson Laboratory (see http://jaxmice.jax.org).

A number of animal models known in the art can be used to examine the activity of DR6 antagonists disclosed herein on neurological disorders such as AD (see, e.g. Rakover et al., Neurodegener Dis. 2007; 4(5): 392-402; Mouri et al., FASEB J. 2007 July; 21(9):2135-48; Minkeviciene et al., J. Pharmacol. Exp. Ther. 2004 November; 311(2):677-82 and Yuede et al., Behav. Pharmacol. 2007 September; 18(5-6):347-63). For example, the effect of DR6 antagonists disclosed herein on the cognitive function of mice can be examined using object recognition tests (see, e.g., Ennaceur et al., Behav. Brain Res. 1988; 31:47-59). The activity of the DR6 antagonists disclosed herein on, for example, brain inflammation, can be examined in mice by for example histochemical analysis as well as ELISA protocols designed to measure levels of inflammation markers such as IL-1β and TNF-α0 and the anti-inflammatory cytokine IL-10 in mouse plasma fractions (see, e.g. Rakover et al., Neurodegener. Dis. 2007; 4(5):392-402).

The effect of the DR6 antagonists disclosed herein on neurological disorders such as Alzheimer's disease (AD) in humans can be examined, for example, through the use of a cognitive outcome measure in conjunction with a global assessment (see, e.g. Leber P: GUIDELINES FOR THE CLINICAL EVALUATION OF ANTIDEMENTIA DRUGS, 1st draft, Rockville, Md., US Food and Drug Administration, 1990). The effects on neurological disorders, such as AD, can be examined for instance using single or multiple sets of criteria. For example, the European Medicine Evaluation Agency (EMEA) introduced a definition of responders corresponding to a prespecified degree of improvement in cognition and stabilization in both functional and global activities (see, e.g. European Medicine Evaluation Agency (EMEA): Note for Guidelines on Medicinal Products in the Treatment of Alzheimer's Disease. London, EMEA, 1997). A number of specific established tests that can be used alone or in combination to evaluate a patient's responsiveness to an agent are known in the art (see, e.g. Van Dyke et al., Am. J. Geriatr. Psychiatry 14:5 (2006). For example, responsiveness to an agent can be evaluated using the Severe Impairment Battery (SIB), a test used to measure cognitive change in patients with more severe AD (see, e.g. Schmitt et al., Alzheimer Dis. Assoc. Disord 1997; 11(suppl 2):51-56). Responsiveness to an agent can also be measured using the 19-item Alzheimer's Disease Cooperative Study—Activities of Daily Living inventory (ADCSADL19), a 19-item inventory that measures the level of independence in performing activities of daily living, designed and validated for later stages of dementia (see, e.g. Galasko et al., J. Int. Neuropsychol Soc. 2005; 11:446-453). Responsiveness to an agent can also be measured using the Clinician's Interview-Based Impression of Change Plus Caregiver Input (CIBIC-Plus), a seven-point global change rating based on structured interviews with both patient and caregiver (see, e.g. Schneider et al., Alzheimer Dis. Assoc. Disord 1997; 11(suppl 2):22-32). Responsiveness to an agent can also be measured using the Neuropsychiatric Inventory (NPI), which assesses the frequency and severity of 12 behavioral symptoms based on a caregiver interview (see, e.g. Cummings et al., Neurology 1994; 44:2308-2314).

Various cholinesterase inhibitors (Donepezil, Galantamine, Rivastigmine and Tacrine as well as Memantine, a N-methyl-D-aspartate (NMDA) receptor antagonist) have received regulatory approval for the treatment of Alzheimer's disease (see, e.g. Roberson et al., Science 314: 781-784 (2006). In clinical trials of cholinesterase inhibitors in patients with AD of mild-to-moderate severity, a common definition of therapeutic response has involved an improvement of at least four-points on the Alzheimer's Disease Assessment Scale-Cognitive Subscale (ADAS-cog) over six months (see, e.g. Winblad et al., Int. J. Geriatr. Psychiatry 2001; 16: 653-666; Cummings J., Am J Geriatr Psychiatry 2003; 11: 131-145; and Lanctot et al., CMAJ 2003; 169: 557-564). These outcomes have also been compared with reversing the disease process by approximately 6 months or 1 year, respectively (see, e.g. Doraiswamy et al., Alzheimer Dis. Assoc. Disord (2001) 15: 174-183). In clinical trials of Memantine, treatment responders have been prespecified as patients who showed no deterioration in global abilities and no deterioration in either functional or cognitive abilities (see, e.g. Reisberg et al., N. Engl. J. Med. 2003; 348: 1333-1341). Another trial of Memantine in patients taking stable doses of the cholinesterase inhibitor Donepezil, characterized Memantine as exhibiting a benefit over placebo on outcome measures including changes from baseline on the Severe Impairment Battery (SIB), and on a modified 19-item AD Cooperative Study-Activities of Daily Living Inventory (ADCS-ADL19), a Clinician's Interview-Based Impression of Change Plus Caregiver Input (CIBIC-Plus), the Neuropsychiatric Inventory (NPI), and the Behavioral Rating Scale for Geriatric Patients (BGP Care Dependency Subscale) (see, e.g. Tariot et al., JAMA 2004; 291:317-324). Memantine has been further characterized as effective by producing both improvement and stabilization of symptoms across multiple SIB, ADCS-ADL19, CIBIC-Plus, and NPI outcome measures (see, e.g. van Dyck et al., Am. J. Geriatr. Psychiatry 14:5 (2006)).

### DR6 Antagonist Diagnostic Applications

Familial Alzheimer's disease (FAD) or Autosomal dominant early onset Alzheimer's disease (ADEOAD) refer to uncommon forms of Alzheimer's disease that usually strike earlier in life, defined as before the age of 65 (usually between 20 and 65 years of age) which can be inherited in an autosomal dominant fashion. Studies of the amyloid precursor protein (APP), presenilin 1 (PSEN1), and presenilin 2 (PSEN2) genes provide evidence that mutations in these genes are responsible for the majority of observed cases of ADEOAD (see, e.g. Raux et al., J. Med. Genet. 2005; 42:793-795). However, a number of observed cases of such syndromes remain unexplained. The data presented herein suggest that polypeptide and/or polynucleotide variants of Death Receptor 6 may be responsible some cases of FAD and/or other neurological disorders. Embodiments of the invention include methods of determining if a polypeptide variant of Death Receptor 6 (DR6) polypeptide comprising SEQ ID NO: 1 is present in an individual, the methods comprising comparing a sequence of a DR6 polypeptide present in the individual with SEQ ID NO: 1 so as to determine if a polypeptide variant of DR6 occurs in the individual. Optionally in such methods, the patient has or is suspected of having a FAD and/or another neurological disorder.

In this context, DR6 polypeptide and/or polynucleotides in patient samples may be analyzed by a number of means well known in the art (e.g. in order to identify naturally occurring variants of DR6), including without limitation, immunohistochemical analysis, in situ hybridization, RT-PCR analysis, western blot analysis of clinical samples and cell lines, and tissue array analysis. Typical protocols for evaluating the sequence of the DR6 gene (e.g. DR6 5′ and 3′ regulatory sequences, introns, exons and the like) and DR6 gene products (e.g. DR6 mRNAs, DR6 polypeptides and the like) can be found, for example in Ausubel et al. eds., 2007, CURRENT PROTOCOLS IN MOLECULAR BIOLOGY, Units 2 (Northern Blotting), 4 (Southern Blotting), 15 (Immunoblotting) and 18 (PCR Analysis).

In an illustrative embodiment of such analyses, neuronal cells are obtained from a patient having a neurological disorder or suspected of being susceptible to a neurological disorder so that the DR6 polypeptide and/or mRNA sequences expressed therein can be analyzed by a procedure such as an immunoassay, a Northern blot assay or a polynucleotide sequence analysis (see, e.g. Lane et al., Laryngoscope 2002; 112(7 Pt 1):1183-9; and Silani et al., Amyotroph Lateral Scler Other Motor Neuron Disord. 200; 2 Suppl 1: S69-76). In certain embodiments of the invention, DR6 polypeptides obtained from patient neuronal cells (which can optionally be passaged in in vitro culture) can be analyzed by an immunoassay such as a Western blot analysis (see, e.g. Pettermann et al., J. Neurosci. (10): 3624-3632 (1988)). Alternatively, a portion of, or the entire coding region of the DR6 gene can be analyzed for example by a reverse transcriptase polymerase chain reaction (RT-PCR) analysis of mRNA extracted from patient neuronal cells. In other embodiments of the invention, DR6 genomic sequences are obtained from a cell other than a neuronal cell, for example a fibroblast or peripheral blood leukocyte and then analyzed to determine if these genomic sequences encode a polypeptide and/or harbor a polynucleotide variant of DR6 (including 5′ and 3′ regulatory sequence variants, for example that influence the levels of DR6 expression in a cell). In certain embodiments of the invention, such analyses can be patterned on analyses of the amyloid precursor protein (APP), presenilin 1 (PSEN1), and presenilin 2 (PSEN2) genes (see, e.g. Nagasaka et al., Proc. Natl. Acad. Sci. USA 2005; 102(41):14854-9; and Finckh et al., Neurogenetics 2005; 6(2):85-9).

### Screening Methods to Identify DR6 Antagonists

Embodiments of the invention include methods of identifying a molecule of interest which inhibits binding of DR6 to APP, the method comprising combining DR6 and APP in the presence or absence of a molecule of interest; and then detecting inhibition of binding of DR6 to APP in the presence of said molecule of interest. In particular, using the disclosure provided herein one can identify proteins, small molecules and other molecules that, for example, interact with DR6 and/or APP and inhibit the interaction between DR6 and APP. In an illustrative embodiment of this method, DR6 can be immobilized on a matrix. The ability of free APP (e.g. APP labelled with a detectable marker such as a chromogenic marker, a fluorescent tag, a radiolabel, a magnetic tag, or an enzymatic reaction product etc.) to bind the immobilized DR6 can then be observed in the presence and absence of a molecule of interest. A decrease in APP binding to DR6 (e.g. as observed via a change in the levels and/or location of the detectable marker) can then be used to identify the molecule as inhibiting the ability of APP to bind DR6. In alternative embodiments of the invention, APP can be immobilized on a matrix in order to detect the ability of APP to bind free DR6 (e.g. DR6 labelled with a detectable marker) in the presence and absence of a molecule of interest. Optionally in such embodiments, the molecule of interest can be an antibody.

The disclosure provided herein allows for a variety of protocols used in the art to characterize the binding between polypeptides such as DR6 and APP to be used to identify a molecule that inhibits the binding interaction between DR6 and APP. Such embodiments of the invention include those that employ ELISA assays (e.g. competition or sandwich ELISA assays as disclosed in U.S. Pat. Nos. 6,855,508; 6,113,897 and 7,241,803), radioimmunoassays (e.g. as disclosed in unit 10.24 of Ausubel et al. eds., CURRENT PROTOCOLS IN MOLECULAR BIOLOGY, 2007), Western blot assays (e.g. as disclosed in Pettermann et al., J. Neurosci. (10): 3624-3632 (1988) and Example 10 below), immunohistological assays (e.g. as disclosed in and Example 10 below), IAsys analyses and CM-5 (BIAcore) sensor chip analyses (see, e.g., U.S. Pat. Nos. 6,720,156 and 7,101,851). In certain embodiments of the invention, a method of identifying a molecule of interest which inhibits binding of DR6 to APP uses a protein microarray. Protein microarrays typically use immobilized protein molecules of interest (e.g. DR6 and/or APP) on a surface at defined locations and have been used to identify small-molecule-binding proteins. (See e.g., Wilson et al., CURR. OPINION IN CHEMICAL BIOLOGY 2001, 6, 81-85; and Zhu, H., et al., Science 2001, 293, 1201-2105).

### Screening Methods to Identify Compounds to Inhibit Neurodegeneration

The invention provides a cell-based screening method to identify compounds that inhibit neurodegeneration. The assays of the examples demonstrate that upon a triggering event for neurodegeneration, APP is shed from the surface of the neurons Inhibitors of this shedding also prevent neurodegeneration. Thus, one can use this readout as an indication of inhibition of neurodegeneration.

The method involves performing an assay in which cells are cultured in the presence or absence of a candidate compound. Upon providing a trigger for neurodegeneration, one compares shedding of APP in the presence of the candidate compound to that observed in the absence of the candidate compound. If the candidate compound inhibits the shedding observed, it is a compound that inhibits neurodegeneration. A trigger for neurodegeration may be any known trigger for neurodegeration, such as but not limited to mechanical disruption, deprivation of nutrients or a trophic factor (e.g., NGF). Examples of the culture conditions and assays to which a candidate compound can be added are shown in the Examples below. In such cases, explants in culture and/or in Campenot chambers may be cultured and the trigger for degeneration (such as NGF deprivation) may be initiated in the presence or absence of the candidate compound.

The candidate compound should inhibit shedding at least 10-30%, 30-50%, 50-70%, 70-90%, or 90-100% inclusive of all whole numbers in between these ranges. Shedding may be observed using antisera against APP such as a polyclonal serum or a monoclonal antibody, such as described in the Examples below. A Bax inhibitor may be added to the medium to prevent non-specific loss of protein due to axonal degeneration.

Compounds identified in such assays are useful in the prevention or treatment of neurological disorders.

### Kits and Articles of Manufacture

In further embodiments of the invention, there are provided articles of manufacture and kits containing materials useful for treating neurological disorders. The article of manufacture comprises a container with a label. Suitable containers include, for example, bottles, vials, and test tubes. The containers may be formed from a variety of materials such as glass or plastic, and are preferably sterilized. The container holds a composition having an active agent which is effective for treating neurological disorders, including Alzheimer's disease. The active agent in the composition is a DR6 antagonist and preferably, comprises anti-DR6 monoclonal antibodies or anti-APP monoclonal antibodies. The label on the container indicates that the composition is used for treating neurological disorders, and may also indicate directions for either in vivo or in vitro use, such as those described above. The article of manufacture or kit optionally further includes a package insert, which refers to instructions customarily included in commercial packages of therapeutic products, that contain information about the indications, usage, dosage, administration, contraindications, other therapeutic products to be combined with the packaged product, and/or warnings concerning the use of such therapeutic products, etc.

The kit of the invention comprises the container described above and a second container comprising a buffer. It may further include other materials desirable from a commercial and user standpoint, including other buffers, diluents, filters, needles, and syringes.

### EXAMPLES

Various aspects of the invention are further described and illustrated by way of the examples that follow, none of which are intended to limit the scope of the invention.

### Example 1

### DR6 Expression in Embryonic and Adult Central Nervous System

RNA in situ screens of TNF receptor superfamily expression patterns in murine embryonic tissues were conducted. More specifically, in situ hybridization experiments were carried out using a mRNA locator Kit (Ambion, Cat. No. 1803) following the manufacturer's protocol. The following primary sequence of DR6 cDNA was used to generate riboprobe for these experiments:

(SEQ ID NO: 3)
GAGCAGAAACGGCTCCTTTATTACCAAAGAAAAGAAGGACACAGTGT
TGCGGCAGGTCCGCCTGGACCCCTGTGACTTGCAGCCCATCTTTGAT
GACATGCTGCATATCCTGAACCCCGAGGAGCTGCGGGTGATTGAAGA
GATTCCCCAGGCTGAGGACAAACTGGACCGCCTCTTCGAGATCATTG
GGGTCAAGAGCCAAGAAGCCAGCCAGACCCTCTTGGACTCTGTGTAC
AGTCATCTTCCTGACCTATTGTAGAACACAGGGGCACTGCATTCTGG
GAATCAACCTACTGGCGG.

A Maxiscript kit (Ambion, Cat. No. 1308) was used for the in vitro synthesis of the riboprobe, according to manufacturer's protocol.

As shown in FIG. 2A, it was found that DR6 was expressed almost exclusively by the differentiated neurons, rather than proliferating progenitors, in developing spinal cord and dorsal root ganglion cells at stages E10 to E12; stages when neuronal cell death is known to occur.

As shown in FIG. 2B, DR6 protein is expressed on both cell bodies and axons of neurons.

In FIG. 2B, the upper two photographs show neurons from a normal mouse visualizing DR6 (left) or a control protein (right). The lower two photographs correspondingly show neurons from a DR6 knock-out mouse visualizing DR6 (left) or a control protein (right).

Materials and methods used to generate the data shown in this figure are as follows. To visualize DR6 protein expression on the sensory axons as shown for example in FIG. 2B, DR6-specific mouse monoclonal antibodies were generated at Genentech using human recombinant DR6 as an immunogen (see Example 3 below). These antibodies were further screened by immunofluorescence for their ability to recognize full-length mouse and human DR6 expressed on the cell surface. One such antibody, termed “3F4.8.8” mAb, and further described in Example 3 and Example 7 below), cross-reacts with both human and mouse DR6 polypeptides, and was used to visualize DR6 expression on axons as shown in FIG. 2B. Immunofluorescence staining procedure was carried out using a standard protocol known in the art (Nikolaev et al., 2003, Cell 112(1), 29-40). To visualize DR6 expression on the axons, pictures were taken on an Axioplan-2 Imaging Zeiss microscope using AxioVision40 Release 4.5.0.0 SP1 (03/2006) computer software from Carl Zeiss Imaging Solutions.

As shown in FIG. 2C, DR6 mRNA is expressed by differentiating neurons. In FIG. 2C from left to right, the three photographs show brain scans of neurons from a normal mouse at developmental stages E10.5, E11.5 and E12.5 respectively.

Materials and methods used to generate the data shown in this figure are as follows. To visualize DR6 mRNA expression in the developing mouse embryo, in situ mRNA hybridization (ISH) with DR63′UTR-specific radio-labeled RNA probe was carried out on 20 micrometer tissue cross sections taken at thoracic axial levels of E10.5-E12.5 mouse embryos. An mRNA locator in situ hybridization kit was used to perform the ISH experiments in accordance with the manufacturer's protocol as outlined in the mRNA locator instruction manual (Ambion Inc., Cat. No. 1803). The radiolabeled mRNA probe corresponding to the anti-sense sequence of mouse DR63′UTR was generated in an in vitro translation reaction using MAXIscript Kit according to manufacturer's instruction manual (Ambion Inc., Cat. No. 1308-1326). DR6 mRNA expression data was visualized using Kodak Autoradiography Emulsion (Kodak) applied to the slides with embryonic tissue cross sections. Pictures were taken in the dark field on the Axioplan-2 Imaging Zeiss microscope using AxioVision40 Release 4.5.0.0 SP1 (03/2006) computer software from Carl Zeiss Imaging Solutions.

The primary sequence of DR6 cDNA used to generate riboprobe in these experiments is as follows:

(SEQ ID NO: 3)
GAGCAGAAACGGCTCCTTTATTACCAAAGAAAAGAAGGACACAGTG
TTGCGGCAGGTCCGCCTGGACCCCTGTGACTTGCAGCCCATCTTTG
ATGACATGCTGCATATCCTGAACCCCGAGGAGCTGCGGGTGATTGA
AGAGATTCCCCAGGCTGAGGACAAACTGGACCGCCTCTTCGAGATC
ATTGGGGTCAAGAGCCAAGAAGCCAGCCAGACCCTCTTGGACTCTG
TGTACAGTCATCTTCCTGACCTATTGTAGAACACAGGGGCACTGCA
TTCTGGGAATCAACCTACTGGCGG

Further analysis using Allen Brain Atlas (http://www.brainatlas.org/aba/; the Allen Brain Atlas is a publicly available scientific resource which provides maps of the expression of approximately 20,000 genes in the mouse brain) revealed that DR6 is highly expressed in cerebral cortex of adult brain. DR6 mRNA is expressed for example in cortical neurons, hippocampal CA1-CA4 pyramidal neurons and the dentate gyrus. DR6 protein is expressed in neuronal cell bodies in the adult cortex and hippocampus.

This pattern of expression provides evidence that, besides its roles in development, DR6 may also function in the progression of neurodegenerative disease associated with neuronal cell loss.

### Example 2

### Inhibition of DR6 Expression by RNA Interference Prevents Axonal Degeneration of Commissural Neurons in Explant Cultures

Commissural neurons are a group of long projection spinal interneurons born in the dorsal spinal cord between developmental stages E9.5 to E11.5. Commissural neurons are believed to be dependent for their survival on trophic support from one of their intermediate targets, the floorplate of the spinal cord. This dependence occurs during a several-day-long period when their axons extend along the floorplate, following which they develop additional trophic requirements. A dependence of neurons on trophic support derived en passant from their intermediate axonal targets provides a mechanism for rapidly eliminating misprojecting neurons, which may help to prevent the formation of aberrant neuronal circuits during the development of the nervous system (Wang et al., Nature, 401:765-769 (1999)).

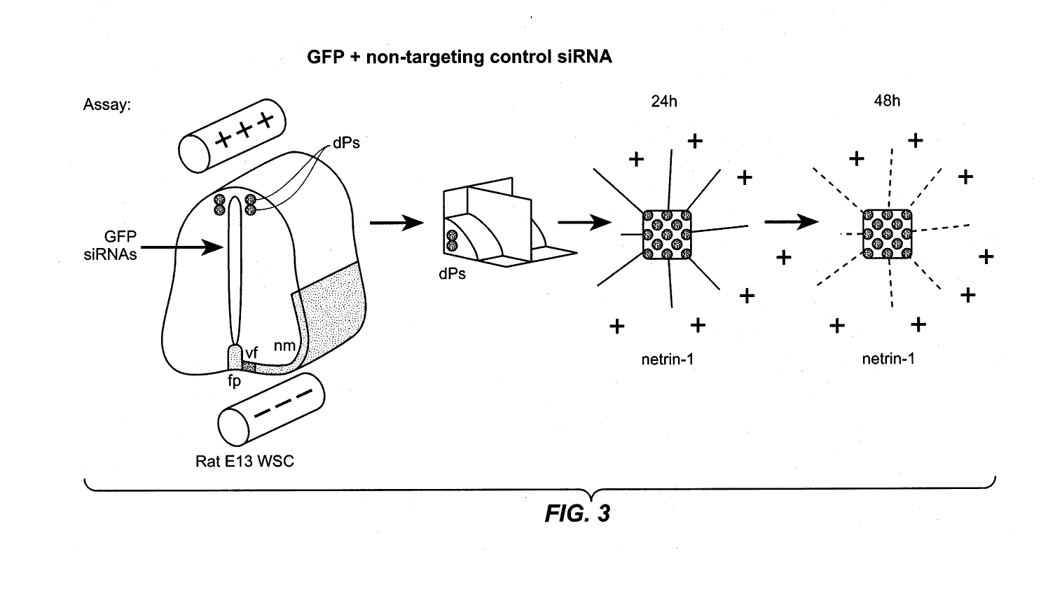

To examine functional roles of DR6 in axonal degeneration and programmed cell death of commissural neurons, an RNAi-based dorsal spinal cord survival assay (Kennedy et al., Cell, 78:425-435 (1994); Wang et al., supra, 1999) was conducted (see FIG. 3). E13 rat or E11.5 mouse embryos were placed in L15 medium (Gibco) and siRNAs (IDT) together with green fluorescent protein (“GFP”)-encoding plasmids were injected into the neural tubes. The siRNAs and plasmids were then delivered to dorsal progenitor cells by electroporation. Dorsal spinal cord explants were dissected out, embedded into a 3D-collagen gel matrix, and cultured in Opti-MEM/F12 medium (Invitrogen) with recombinant netrin-1 (R&D Pharmaceuticals) and 5% horse serum (Sigma) at 37° C. in a 5% CO₂ environment. Within 16 hours in response to chemo-attractant netrin-1, commissural axons grow out of the explant into the collagen matrix gel (Kennedy et al., supra, 1994). Commissural axons are visualized by GFP fluorescence by observation using an inverted microscope.

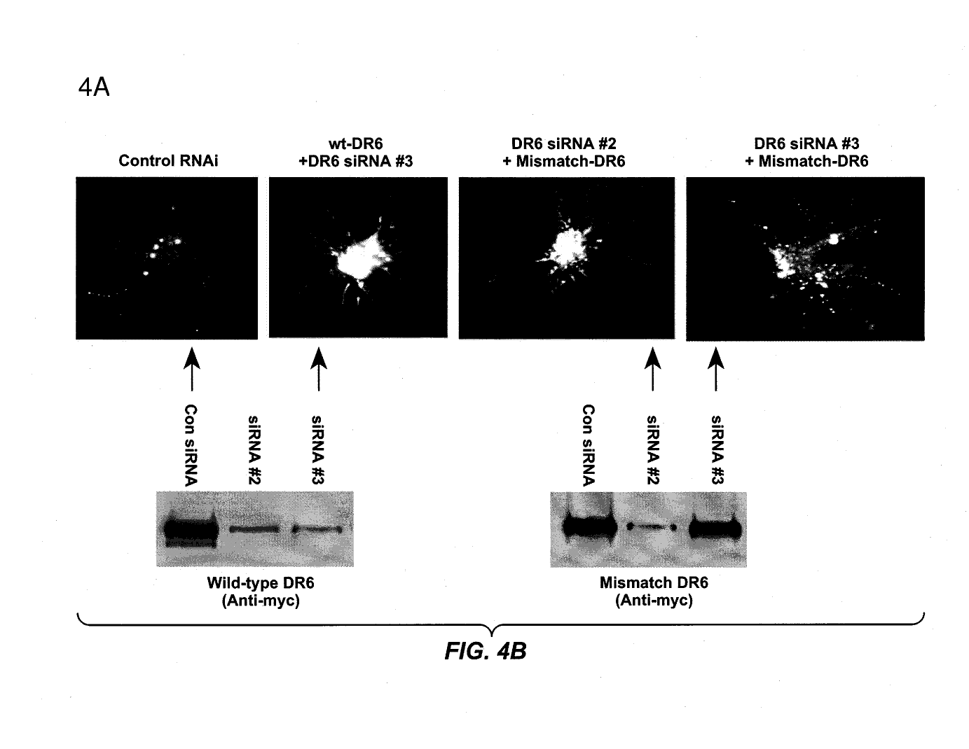

As shown in FIG. 4A, after 48 hours in culture in the absence of trophic factor support derived from the floorplate, commissural neurons undergo programmed cell death and their axons degenerate (see, also, Wang et al., supra, 1999). Such axonal degeneration was markedly blocked when DR6 expression in the commissural neurons was down-regulated by DR6-specific siRNA molecules (see, FIG. 4, lower panel). This inhibition of axonal degeneration was not observed in control experiments with non-targeting siRNA molecules. The data suggests that DR6 is an important pro-apoptotic receptor required for axonal degeneration of commissural neurons upon withdrawal of trophic support from their intermediate target, the floorplate of the spinal cord.

As shown in FIG. 4B, an RNAi-resistant DR6 cDNA rescues the degeneration phenotypes blocked by DR6 siRNA.

In FIG. 4B from left to right, the upper four photographs show neurons in the presence of: (1) a control RNAi; (2) wild type-DR6 exposed to DR6 siRNA #3; (3) a mismatch-DR6 exposed to DR6 siRNA #2; and (4) a mismatch-DR6 exposed to DR6 siRNA #3. The lower two panels show autoradiograms of: (1) wild-type DR6 mRNA in the presence of: control siRNA, siRNA#2, and siRNA#3; and (2) mismatch DR6 mRNA in the presence of: control siRNA, siRNA#2, and siRNA#3.

Materials and methods used to generate the data shown in this figure are as follows. To investigate physiological roles of DR6 receptor in axonal degeneration and programmed cell death of commissural neurons, a dorsal spinal cord survival assay according to protocols known in the art (Kennedy et al., Cell 78:425-435 (1994); Wang et al., Nature 401:765-769 (1999)) was performed (with data shown in FIG. 4B). E13 rat embryos were placed in L15 medium (Gibco) and injected into their neural tubes with the following siRNA constructs (FIG. 4B):

(SEQ ID NO: 11)
5′-AAUCUGUUGAGUUCAUGCCUU-3′
(SEQ ID NO: 12)
5′-CAAUAGGUCAGGAAGAUGGCU-3′
(SEQ ID NO: 13)
5′-GGACTCTGTGTACAGTCACCTCCCAGATCTGTTATAG-3′

Control non-targeting, or targeting DR6 siRNA #2, or targeting DR6 siRNA #3 (IDT) together with either wild-type or mis-match DR6 cDNA and GFP-encoding plasmids. DR6 cDNA and GFP cDNA were subcloned into pCAGGS vector backbone (commercially available from BCCM/LMBP). siRNAs and plasmids were then delivered to dorsal progenitor cells by electroporation. Dorsal spinal cord explants were then dissected out, embedded into a 3D collagen gel matrix and cultured in Opti-MEM/F12 medium (Invitrogen) with recombinant netrin-1 and 5% horse serum (Sigma) at 37° C. in a 5% CO₂ environment. Within 16 hours in response to chemo-attractant netrin-1 commissural axons grow out of the explant into the collagen matrix gel (Kennedy et al., Cell 78:425-435 (1994). Commissural axons are visualized by GFP fluorescence by observation using inverted microscope. After 48 hours in culture in the absence of trophic factor support derived from the floorplate, commissural neurons undergo programmed cell death and their axons degenerate (Wang et al., Nature, 401:765-769 (1999)) (FIG. 4B). However, the axonal degeneration program can be blocked by introduction of a targeting DR6-specific siRNA #3 (FIG. 4B). The specific, on-target effect of DR6-specific siRNA #3 is further confirmed in a rescue experiment in which axonal degeneration phenotype is restored by co-expression of the siRNA#3-resistant mis-match DR6 cDNA construct together with DR6 siRNA #3 (FIG. 4B). Presented experimental evidence establishes that DR6 receptor function is required for axonal degeneration and death of commissural neurons upon withdrawal from their intermediate target, the floorplate of the spinal cord.

The sequences of DR6 siRNAs #2 and #3 (sense strands), and the mismatch fragment of DR6 cDNA complementary to DR6 siRNA #3 sequence used in the above described assay are as follows:

| (SEQ ID NO: 11) | |
|---|---|
| Rat DR6 siRNAs #2: | 5′-AAUCUGUUGAGUUCAUGCCUU-3′ |
| (SEQ ID NO: 12) | |
| Rat DR6 siRNAs #3: | 5′-CAAUAGGUCAGGAAGAUGGCU-3′ |

Mismatch fragment of rat DR6 cDNA complementary to DR6 siRNA #3 sequence:

(SEQ ID NO: 13)
5′-GGACTCTGTGTACAGTCACCTCCCAGATCTGTTATAG-3′

### Example 3

### Inhibition of DR6 Receptor Signaling By Anti-DR6 Antibodies Prevents Axonal Degeneration of Commissural Neurons in Explant Cultures

A dorsal spinal cord survival assay (as described in Example 2 above) was conducted using anti-DR6 antibodies. Microscopic observation (using green fluorescence channel for GFP) was employed to visualize commissural axons. The dorsal spinal cord survival assay was carried out according to protocols known in the art (Kennedy et al., Cell 78:425-435 (1994); Wang et al., Nature 401:765-769 (1999)) with modifications outlined in the Example 2 above. E13 rat embryos were injected into their neural tubes with the GFP-expressing plasmid construct (GFP cDNA were subcloned into pCAGGS vector backbone, commercially available from BCCM/LMBP). GFP-expressing plasmid was then delivered to dorsal progenitor cells by electroporation. Anti-DR6 blocking antibodies or control normal mouse IgG were added to commissural explants at 40 ug/ml 24 hours after plating. Pictures of the commissural explants were taken 48 hours after plating as outlined below. To visualize GFP-expressing commissural axons, pictures were taken on the Axiovert 200 Zeiss inverted microscope (in green fluorescence channel for GFP) using AxioVision40 Release 4.5.0.0 SP1 (March 2006) computer software from Carl Zeiss Imaging Solutions.

The anti-DR6 antibodies used for this experiment were generated as follows.

A human DR6 extracellular domain sequence fused with Fc (hDR6-ECD-Fc) was used as an immunogen to generate anti-DR6 mouse monoclonal antibodies. The sequence of the hDR6-ECD-Fc immunogen used is as follows:

(SEQ ID NO: 4)
MGTSPSSSTALASCSRIARRATATMIAGSLLLLGFLSTTTAQPEQKA
SNLIGTYRHVDRATGQVLTCDKCPAGTYVSEHCTNTSLRVCSSCPVG
TFTRHENGIEKCHDCSQPCPWPMIEKLPCAALTDRECTCPPGMFQSN
ATCAPHTVCPVGWGVRKKGTETEDVRCKQCARGTFSDVPSSVMKCKA
YTDCLSQNLVVIKPGTKETDNVCGTLPSFSSSTSPSPGTAIFPRPEH
METHEVPSSTYVPKGMNSTESNSSASVRPKVLSSIQEGTVPDNTSSA
RGKEDVNKTLPNLQVVNHQQGPHHRHILKLLPSMEATGGEKSSTPIK
GPKRGHPRQNLHKHFDINEHLPWMIPDKTHTCPPCPAPELLGGPSVF
LFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKT
ISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMH
EALHNHYTQKSLSLSPGK

The fusion polypeptide was generated using immunoadhesin protocols previously described (Ashkenazi et al., Curr Opin Immunol. 9(2):195-200 (1997); Haak-Frendscho et al., J. Immunol. 152(3):1347-53 (1994)).

The 9 week old-Balb/c mice were immunized by injection with 100 ul of hDR6-ECD-Fc immunogen (1 mg/animal) over the course of an approximately eight-week period. Lymph nodes (11×10⁶ cell/ml, 5 ml) of all the immunized mice were then fused with PU.1 myeloma cells (murine meyloma cells from ATCC) at a concentration of 5×10⁶ cells/ml, 5 ml. Cells were plated into 4 plates at 2×10⁶ cells/ml.

A capture ELISA was used to screen hybridomas for specificity binding to the hDR6-ECD-Fc polypeptide described above. Plates were coated with 50 ul of 2 ug/ml goat anti-human IgG Fc specific (Cappel Cat. No. 55071) at 4° C. over-night. Plates were washed three times with PBS plus Brij, and plates were blocked with 200 ul of 2% BSA at room temperature for 1 hour. Plates were then washed three times with PBS plus Brij. Subsequently, the plates were incubated with 100 ul/well immunoadhesin at 0.4 ug/ml for 1 hour on a shaker. Plates were then washed three times with PBS plus Brij. 100 ul of 1^{st} antibodies were added to wells, incubated for 1 hour on shaker. Plates were again washed three times with PBS plus Brij. 100 ul of sheep anti-mouse IgG HRP (no cross to human, Cappel Cat. No. 55569) antibody at 1:1000 for 1 hour. Plates were washed three times with PBS plus Brij. 50 ul of substrate (TMB Microwell peroxidase KPL #50-76-05) was added and plates were incubated for 5 minutes. Reaction was stopped with 50 ul/well of stop solution (KPL #50-85-05). Absorbance was read at 450 nm. The assay buffer used contained PBS, 5% BSA, and 0.05% Tween 20.

Hybridomas that tested positive in the binding to the hDR6-ECD-Fc polypeptide in the capture ELISA assay were then cloned by limiting dilution (SCDME media containing 10% HCF, 10% FCS). 10 days later plates were taken out and wells with one colony were assayed by the capture ELISA described above. Various selected monoclonal antibodies were then isotype tested, and were shown to be of the IgG1 isotype.

Four of the anti-DR6 mAbs, identified as “3B11.7.7”; “3F4.4.8”; “4B6.9.7”; and “1E5.5.7”, were then tested in the dorsal spinal cord survival assay for their ability to block axonal degeneration.

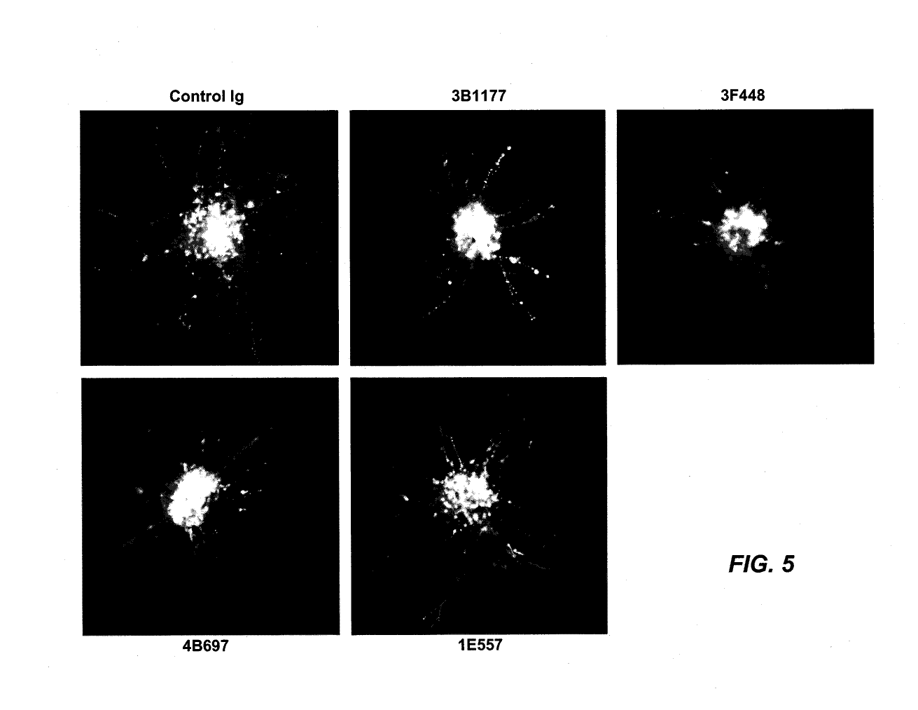

Strikingly, certain of these anti-DR6 mAbs (3F4.4.8; 4B6.9.7; and 1E5.5.7) were able to partially inhibit axonal degeneration of commissural neurons induced by trophic deprivation for 48 hours in culture (see FIG. 5). It is believed that such antibodies may promote neuronal survival, for instance, by blocking the interaction between putative DR6 ligand and DR6 receptor or by inhibiting ligand-independent DR6 signaling. The 3B11.7.7 DR6 antibody had a slight stimulatory effect in inducing axonal degeneration.

### Example 4

### Inhibition of DR6 Receptor Signaling by Specific Peptide Inhibitor of Jun N-Terminal Kinase (JNKi)

The DR6 receptor has been reported to signal through activation of JNK, and JNK activity was observed to be impaired in a DR6 null mouse model (Pan et al., FEBS Lett., 431:351-356 (1998); Zhao et al., Journal of Experimental Medicine, Vol. 194, 1441-1441, 2001)). To examine roles of DR6-JNK signaling in axonal degeneration, a dorsal spinal cord survival assay (as described in Example 2 above) was conducted except that the JNK signaling pathway was blocked in commissural neurons by using a peptide inhibitor, L-JNK-I ((L)-HIV-TAT48-57-PP-JBD20; Calbiochem) at 1 μM concentration. DMSO (SIGMA) and normal mouse IgG were tested as controls.

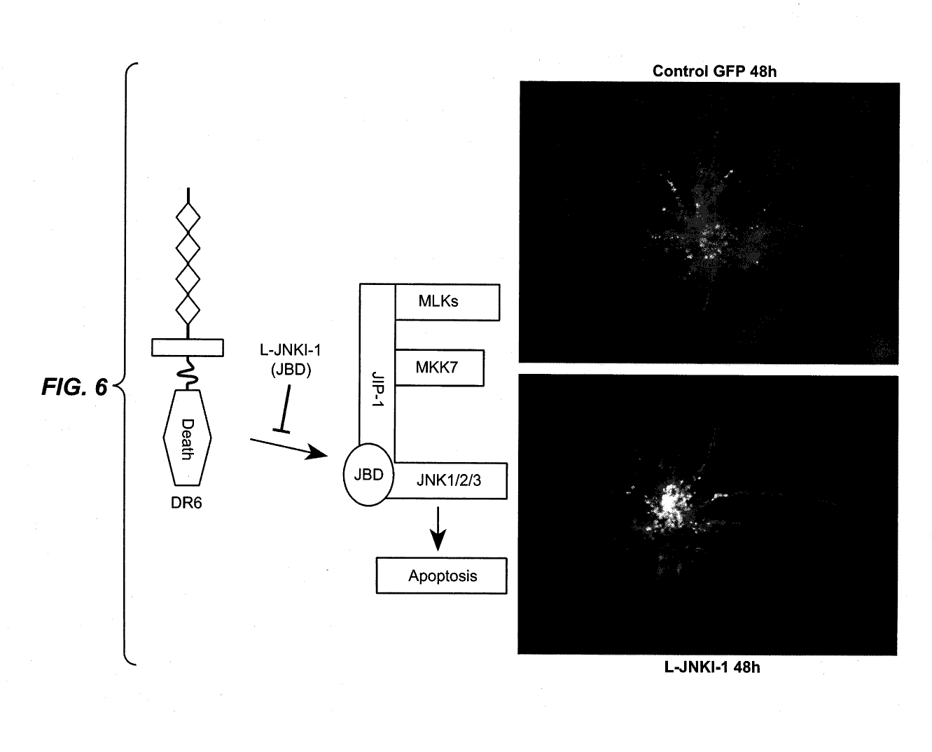

As shown in FIG. 6, this inhibition of JNK signaling partially blocked axonal degeneration in the dorsal spinal cord survival assay. The data suggests that DR6 signals degeneration of axonal processes at least in part through the JNK pathway.

### Example 5

### Inhibition of DR6 Receptor Signaling by Anti-DR6 Antibodies Prevents Neuronal Cell Death in Mouse Embryonic Spinal Cords

Assays were conducted wherein DR6 signaling was blocked by anti-DR6 mAbs in a whole embryo culture system. This system, described below, allows whole mouse embryos to be cultured in vitro in vials for 2 days from the developmental stage E9.5 to E11.5. E9.5 embryos were dissected out of uterus with yolk sac attached to the embryo and cultured in 100% rat serum (Harlan) in a 65% oxygen environment for the first day and 95% oxygen for the second day at 37° C. Anti-DR6 mAbs (described in the Examples above) were added in the assays at a final concentration of 10 μg per ml, and normal mouse IgG antibody at concentrations of 10 μg per ml were used as controls.

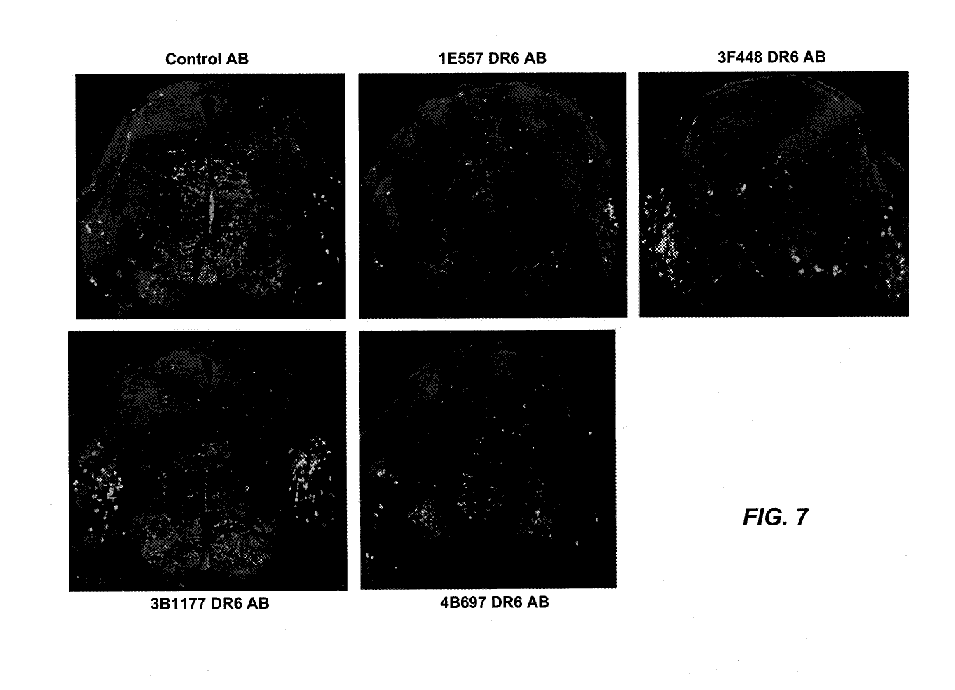

Immunofluorescence staining with antibody recognizing cleaved Caspase-3 (antibody to mouse cleaved Caspase-3, purchased from R&D Systems) was used to detect and microscopically observe the apoptotic cells. The results are illustrated in FIG. 7. Strikingly, inhibition of DR6 by the anti-DR6 mAbs 3F4.4.8; 4B6.9.7; and 1E5.5.7 protected spinal cord neurons against naturally occurring developmental cell death in this system.

### Example 6

### Reduced Neuronal Cell Death in DR6 Null Mice

Phenotypes of DR6 knockout embryos (Zhao et al., J. Exp. Med. 194:1441-1441, 2001) at developmental stage E15.5 were analyzed. Cleaved caspase 3 is a marker of apoptotic cells, and to examine the extent of neuronal cell death in embryonic spinal cords, immunostaining for cleaved caspase 3 (antibody to mouse cleaved Caspase-3, purchased from R&D Systems) was used. DR6 heterologous litter mates were also examined as controls. Paraformaldehyde (PFA)-fixed embryonic tissue sections were blocked for 1 hour in blocking solution (2% heat-inactivated goat serum (Sigma)/PBS (Gibco)/0.1% Triton (Sigma)) and incubated overnight at 4° C. with primary antibody (1:500 dilution of antibody to mouse cleaved Caspase-3, purchased from R&D Systems) in blocking solution. Sections were washed three times by blocking solution for 1 hour at room temperature and incubated with secondary antibody (1:500 dilution of goat anti-rabbit Alexa 488, Molecular Probes, Invitrogen) for 1 hour at room temperature. Sections were then washed for 1 hour at room temperature by blocking solution and visualized by immunofluorescence in green channel.

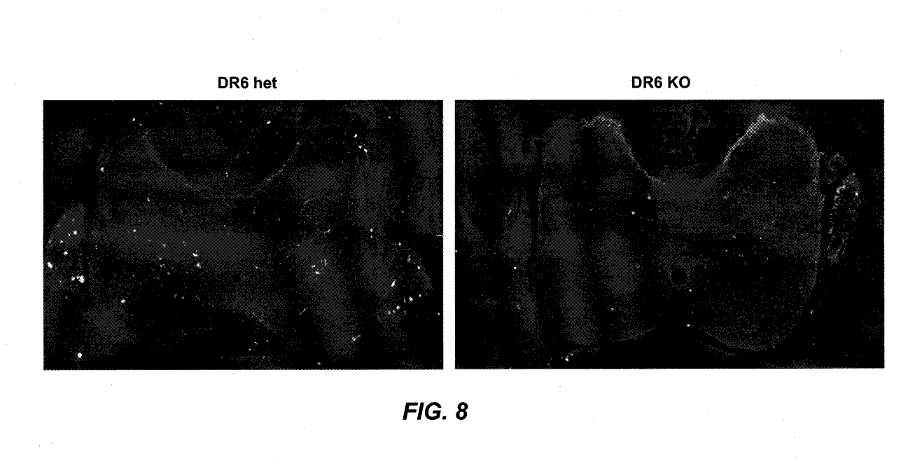

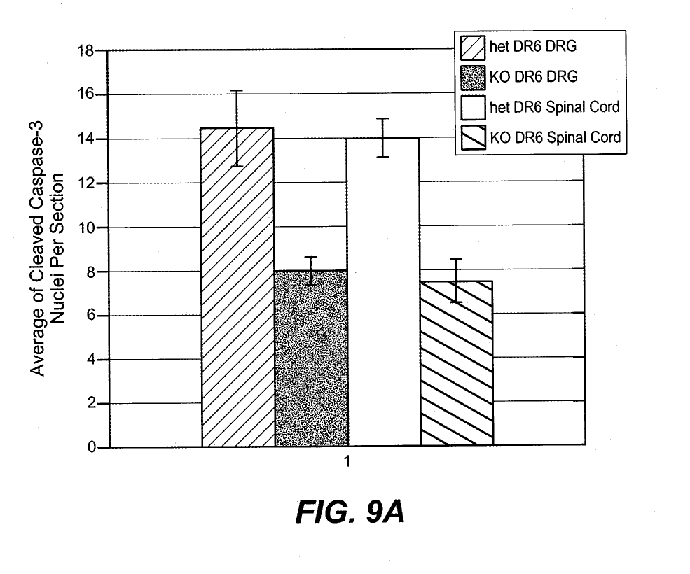

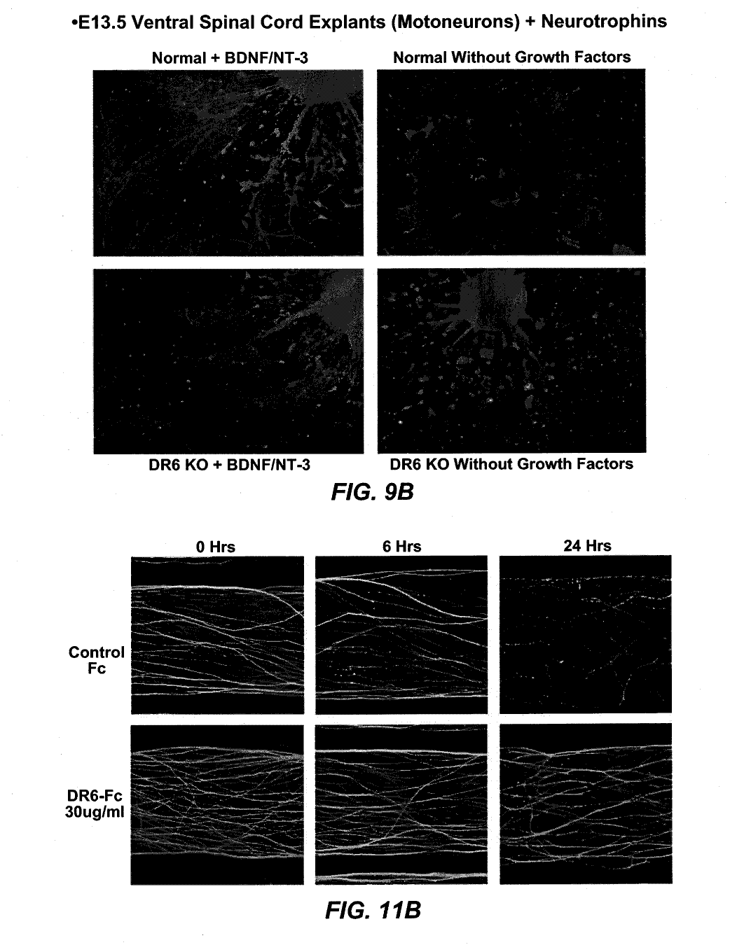

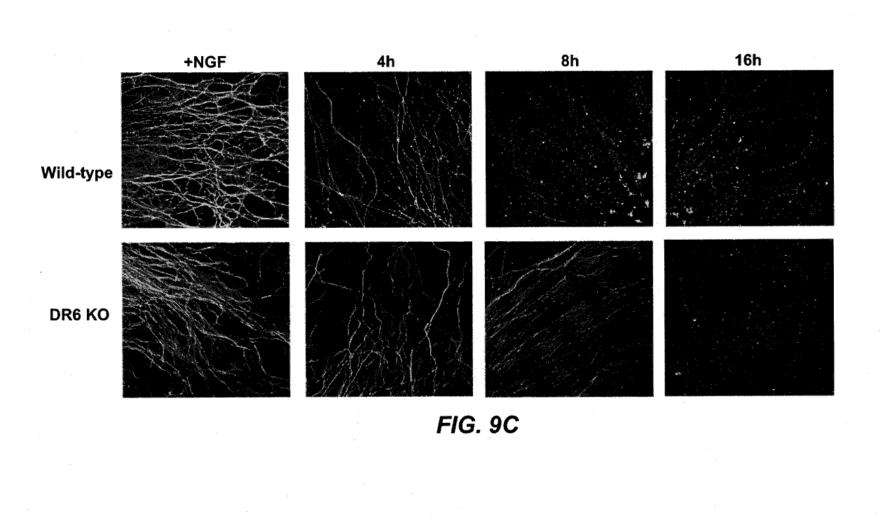

The number of caspase 3 positive nuclei per spinal cord section per embryo was quantified (see FIGS. 8 and 9A). An approximately 40 to 50% reduction in neuronal cell death was detected in DR6 null mice spinal cords and dorsal root ganglions (“DRGs”) as compared to DR6 heterozygous littermate controls (FIGS. 8 and 9A). Accordingly, it is believed that DR6 signaling may promote neuronal cell death in the developing nervous system in vivo.

As shown in FIG. 9B, DR6 is required for motor axon degeneration as verified with DR6 null mice. Ventral spinal cord explants (motor neurons) from normal as well as DR6 knockout embryos (Zhao et al., Journal of Experimental Medicine, Vol. 194, 1441-1441, 2001) at developmental stage E13.5 were analyzed in the presence and absence of brain-derived neurotrophic factor (BDNF) and neurotrophin 3 (NT-3) (BDNF and NT-3 obtained from Chemicon).

In FIG. 9B, the upper left panel shows ventral spinal cord explants from normal mice in the presence of BDNF and NT-3, while the lower left panel shows ventral spinal cord explants from DR6 knock out (KO) mice in the presence of BDNF and NT-3. Similarly, the upper right panel shows ventral spinal cord explants from normal mice in the absence of these growth factors and the lower right panel shows ventral spinal cord explants from DR6 knock out (KO) mice in the absence of these growth factors.

Materials and methods used to generate the data shown in this FIG. 9B are as follows. The motor neuron ventral spinal cord survival assay was carried out as described in Henderson et al., Nature 363:266-270 (1993) with a few modifications. DR6 heterozygous or DR6 null mouse E13.5 embryos were dissected out using alcohol-treated scissors and placed in warm L15 medium (Gibco). Using the same scissors and forceps, ventral region of the embryo was opened up, organs were removed, ribs were cut away and whole spinal cord was dissected out, the surrounding meninges tissue was than removed with forceps. Roof plates were removed and the open book prep of spinal cord was obtained. The ventral half of the spinal cord including MMC and LMC motor columns was isolated and the remaining floorplate tissue was carefully cut away. Ventral spinal cords were transferred with yellow tips that have been coated in L15 to new small dish w/L15+5% FBS (Sigma) serum for further sectioning into explants using a tungsten needle.

PDL/Laminin coated 8 well slides (Becton, Dickinson and Company) were filled with 500 μl per well Neurobasal Medium (Invitrogen) plus 50 ng/ml of each recombinant BDNF and NT-3 (Chemicon), plus B-27 supplement X50 (Invitrogen); plus Pen Strip Glutamine X100 (Cat. No. 10378-016; Gibco) plus Glucose X100. Sectioned ventral spinal cord explants were placed in each well (2-3 explants per well) and placed in a 37° C. incubator for 48 hours for growth. Two days later, trophic factor deprivation was carried out as follows: old medium was taken away, and the wells were gently washed twice with Neurobasal medium (WITHOUT trophic factors).

Pre-warmed Neurobasal Medium/B-27 (Invitrogen) (prepared as above described WITHOUT trophic factors) plus anti-BDNF and anti-NT3 blocking antibodies (Genentech, Inc.) were added at 20 ug/ml. Slides with explants were then incubated at 37° C. for another 24-48 hours.

Two days later, explants were fixed in 4% PFA in PBS, permeabilized with 0.2% Triton in Net Gel (Nikolaev et al., 2003, Cell 112(1), 29-40) for 10 minutes at 0° C., and washed twice with Net Gel. To block non-specific binding sites, slides were incubated in 1% BSA in PBS, at 4° C. overnight. To visualize degenerating motor axons, immunostaining with anti-p75NTR-specific antibody (1:500 dilution, Chemicon) was carried out the following day (primary Ab 1:500 overnight 4° C. in 1% BSA/PBS, secondary Ab 1:500 for 1 hour at room temperature). Wells were pulled off, and Fluoromount-G was used to mount slides with cover slips. To visualize p75NTR-expressing motor axons, pictures were taken on the Axioplan-2 Imaging Zeiss microscope using AxioVision40 Release 4.5.0.0 SP1 (03/2006) computer software from Carl Zeiss Imaging Solutions.

As shown in the data disclosed in FIG. 9C, injury induced degeneration is delayed in DR6 knock-out mice.

In FIG. 9C from left to right, the upper 4 panels show neurons from normal mice: in the presence of nerve growth factor (NGF); and 4, 8 or 16 hours post injury, respectively. In FIG. 9C from left to right, the lower 4 panels from left to right show neurons from DR6 KO mice: in the presence of exogenous nerve growth factor (NGF); and 4, 8 or 16 hours post-injury, respectively.

The in vitro sensory axon lesion assay as shown in FIG. 9C was carried out as follows. DR6 heterozygous or DR6 null mouse E12.5 embryos were dissected out and placed in warm L15 medium (Gibco). Using the same scissors and forceps, ventral region of the embryo was opened up, organs were removed, ribs were cut away and dorsal root ganglions (DRGs), attached to the spinal cord, were dissected out with forceps. DRGs were then transferred with yellow tips that have been coated in L15 to new small dish w/L15+5% FBS (Sigma) serum for further sectioning into ¼ DRG explants using a tungsten needle.

PDL/Laminin pre-coated plastic 8 well slides (Becton, Dickinson and Company) were filled with 500 ul per well Neurobasal Medium (Invitrogen) plus 50 ng/ml of NGF (Roche Molecular Biochemicals), plus B-27 supplement X50 (Invitrogen); plus Pen Strip Glutamine X100; plus Glucose X100. Sectioned DRG explants were placed in each well (2-3 DRG explants per well) and placed in a 37° C. incubator for 48 hours for growth. Two days later, an axon lesion assay was carried out as follows: injury was induced by making two parallel cuts of sensory axons just above and just below the DRG explant with a micro-knife (Fine Science Tools). The uncut axons to the left and to the right of the DRG explants served as endogenous no lesion controls. Slides with cut DRG explants were fixed 0, 4, 8, 16 and 24 hours post-injury, in 4% PFA in PBS, permeabilized with 0.2% Triton in Net Gel (Nikolaev et al., 2003, Cell 112(1), 29-40) for 10 minutes at 0° C., and washed twice with Net Gel. To block non-specific binding sites, slides were incubated in 1% BSA in PBS, at 4° C. for overnight. To visualize degenerating sensory axons, immunostaining with a Neuronal Class III β-Tubulin (TUJ1)-specific antibody (1:500 dilution, Covance) was carried out the following day (primary Ab 1:500 overnight 4° C. in 1% BSA/PBS, secondary Ab 1:500 for 1 hour at room temperature). Wells were pulled off, and Fluoromount-G was used to mount slides with cover slips. To visualize sensory axons labeled with immunofluorescence, pictures were taken on the Axioplan-2 Imaging Zeiss microscope using AxioVision40 Release 4.5.0.0 SP1 (March 2006) computer software from Carl Zeiss Imaging Solutions.

### Example 7

### Anti-DR6 Antibody Antagonists Inhibit Degeneration of Neurons

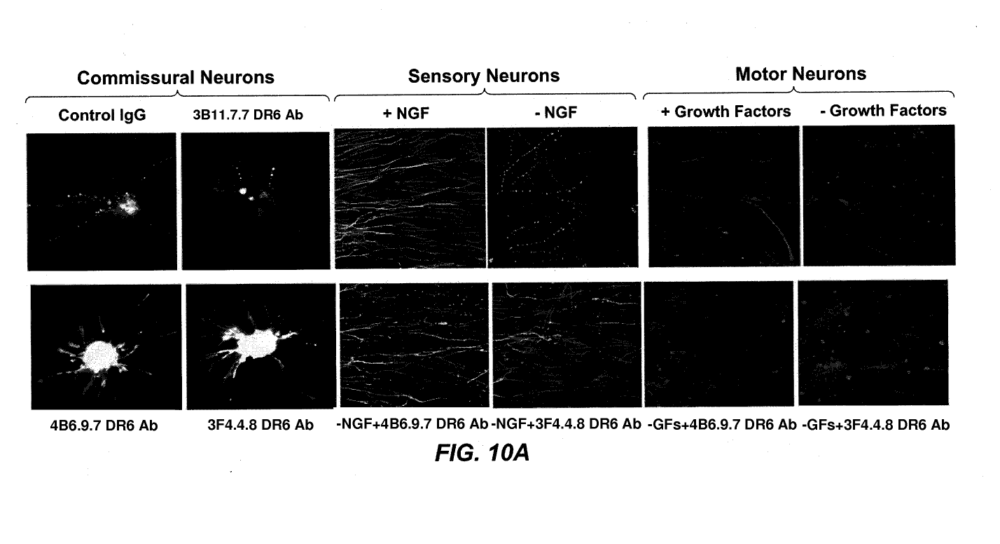

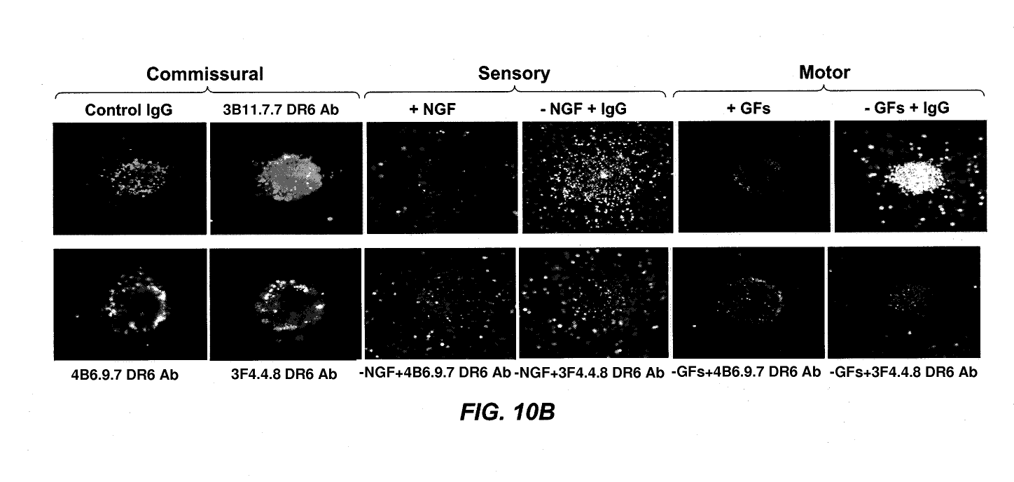

As shown in FIG. 10A, anti-DR6 antibodies inhibit degeneration of diverse trophic factor deprived neurons (in assays of axonal degeneration).

In FIG. 10A from left to right, the first two upper and lower photographs show data from commissural neurons. In these first four photographs, the upper two photographs show commissural neurons in the presence of a control IgG and the 3B11.7.7 DR6 antibody respectively, while the lower two photographs show commissural neurons in the presence of 4B6.9.7 DR6 antibodies and the 3F4.4.8 DR6 antibodies, respectively. The middle two upper and lower photographs in FIG. 10A show data from sensory neurons. In these middle four photographs, the upper two photographs show sensory neurons in the presence and absence of NGF respectively, while the lower two photographs show sensory neurons in the absence of NGF, but in the presence of 4B6.9.7 DR6 antibodies and 3F4.4.8 DR6 antibodies, respectively. The two upper and lower photographs on the right side of FIG. 10A show data from motor neurons. In these right four photographs, the upper two photographs show motor neurons in the presence and absence of growth factors respectively, while the lower two photographs show motor neurons in the absence of growth factors, but in the presence of 4B6.9.7 DR6 antibodies and 3F4.4.8 DR6 antibodies, respectively.

Materials and methods used to generate the data shown in this figure are as follows. The mouse monoclonal 4B6.9.7, IE5.5.7, 3F4.4.8, 2C7.3.7 and 3B11.7.7 DR6 antibodies were generated by immunizing a mouse with DR6 ectodomain as described in the Example 3 above.

The sensory, motor, and commissural explant cultures were carried out as in the above described Example 2 and Example 6, with modifications as follows. For the commissural explant survival assay, DR6 antibodies 4B6.9.7 or 3F4.4.8, or control IgG, were added to commissural explant cultures at 20 micrograms/ml final concentration 24 hours after plating (FIG. 10A). For sensory explant cultures, the NGF deprivation assay was carried out 48 hours after plating. Fresh neurobasal medium without NGF, but with NGF-blocking antibody (Genentech, Inc.) together with the indicated DR6 antibodies (4B6.9.7 or 3F4.4.8) or control IgG were added to sensory explant cultures at 20 micrograms/ml final concentration 48 hours after plating (FIG. 10A). For motor explant cultures, a trophic factor deprivation assay was carried out 48 hours after plating. Fresh neurobasal medium without NT3/BDNF, but with BDNF-blocking and NT3-blocking antibodies (function blocking trophic factor mAbs, Genentech, Inc.) together with indicated DR6 antibodies (4B6.9.7 or 3F4.4.8) or control IgG were added to sensory explant cultures at 20 micrograms/ml final concentration 48 hours after plating (FIG. 10A). To visualize sensory and motor axons that were labeled by immunofluorescence staining with anti-TUJ1 (Covance) and anti-p75NTR (Chemicon/Millipore) antibodies accordingly, pictures were taken on the Axioplan-2 Imaging Zeiss microscope using AxioVision40 Release 4.5.0.0 SP1 (03/2006) computer software from Carl Zeiss Imaging Solutions. To visualize GFP-expressing commissural axons, pictures were taken on the Axiovert 200 Zeiss inverted microscope (in green fluorescence channel for GFP) using AxioVision40 Release 4.5.0.0 SP1 (03/2006) computer software from Carl Zeiss Imaging Solutions.

As shown in FIG. 10B, the anti-DR6 antibodies inhibited degeneration of diverse trophic factor-deprived neurons (in assays of apoptosing cell bodies via a TUNEL stain). In FIG. 10B starting from the left, the two upper and lower photographs show data from commissural neurons. In these first four photographs, the upper two photographs show commissural neurons in the presence of a control IgG and the 3B11.7.7 DR6 antibody, respectively, while the lower two photographs show commissural neurons in the presence of 4B6.9.7 DR6 antibodies and the 3F4.4.8 DR6 antibodies, respectively. The middle set of two upper and lower photographs in FIG. 10B show data from sensory neurons. In these middle four photographs, the upper two photographs show sensory neurons in the presence and absence of NGF respectively, while the lower two photographs show sensory neurons in the absence of NGF, but in the presence of 4B6.9.7 DR6 antibodies and 3F4.4.8 DR6 antibodies, respectively. The set of two upper and lower photographs on the right side of FIG. 10B show data from motor neurons. In these right four photographs, the upper two photographs show motor neurons in the presence and absence of growth factors respectively, while the lower two photographs show motor neurons in the absence of growth factors, but in the presence of 4B6.9.7 DR6 antibodies and 3F4.4.8 DR6 antibodies, respectively.

The disclosure in FIG. 10 suggests that ligand may play an important role for DR6 function in axonal degeneration.

Materials and methods used to generate the data shown in this figure are as follows. As noted above, the mouse monoclonal 4B6.9.7, IE5.5.7, 3F4.4.8, 2C7.3.7 and 3B11.7.7 DR6 antibodies were generated by immunizing a mouse with DR6 ectodomain as described in the Example 3 above. The sensory, motor, and commissural explant cultures were carried out as in the above described Example 2 and Example 6, with modifications outlined as follows. For the commissural explant survival assay, as described in Example 3 above, DR6 antibodies 4B6.9.7 and 3F4.4.8, antibody 3B11.7.7 (alternatively referred to as control IgG (Genentech, Inc.)), were added individually to commissural explant cultures at 20 micrograms/ml final concentration 24 hours after plating (FIG. 10B, left).

For sensory explant cultures, the NGF deprivation assay was carried out 48 hours after plating. Fresh neurobasal medium without NGF, but with NGF-blocking antibody (Genentech, Inc.) together with DR6 antibodies 4B6.9.7 or 3F4.4.8, or control IgG (Genentech, Inc.) were added to sensory explant cultures at 20 micrograms/ml final concentration 48 hours after plating (FIG. 10B, middle). For motor explant cultures, a trophic factor deprivation assay was carried out 48 hours after plating. Fresh neurobasal medium without NT3/BDNF, but with BDNF-blocking and NT3-blocking antibodies (function blocking trophic factor mAbs, Genentech, Inc.) together with 4B6.9.7 or 3F4.4.8, or control IgG (Genentech, Inc.) were added to sensory explant cultures at 20 micrograms/ml final concentration 48 hours after plating (FIG. 10B, right).

Explants were fixed in 4% PFA/PBS and processed for the detection of apoptosis at single cell level, based on labeling of DNA strand breaks (TUNNEL technology) using the In situ Cell Death Detection Kit (Cat. No. 11 684 795 910, Roche) according to manufacturer's instructions manual (Roche). Apoptosis in cell bodies of commissural sensory and motor explant cultures was analyzed by fluorescence microscopy (FIG. 10B). To visualize fluorescently labeled TUNNEL-positive apoptotic cell bodies, pictures were taken on the Axioplan-2 Imaging Zeiss microscope (in red fluorescence channel) using AxioVision40 Release 4.5.0.0 SP1 (03/2006) computer software from Carl Zeiss Imaging Solutions.

### Example 8

### DR6 Immunoadhesin Antagonists Inhibit Degeneration of Neurons

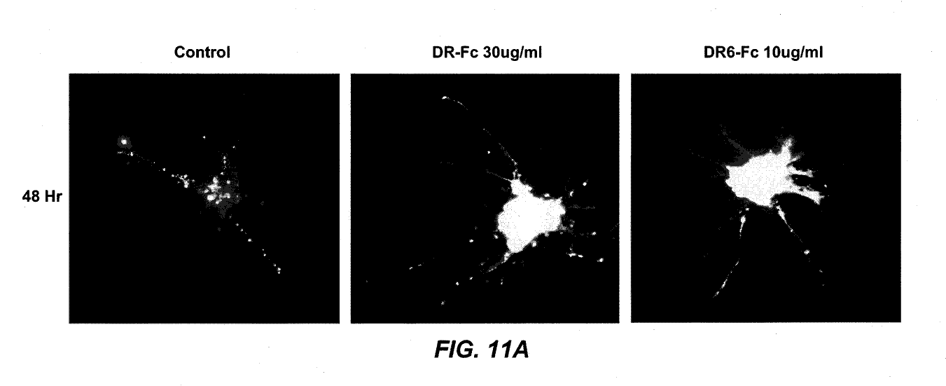

As shown in FIG. 11A, commissural axon degeneration was delayed by hDR6-ECD-Fc. The hDR6-ECD-Fc immunoadhesin protein used in this assay is described above in Example 3.

In FIG. 11A from left to right, the first photograph provides a control showing commissural axon degeneration at 48 hours. The second photograph shows commissural axon degeneration at 48 hours in the presence of 30 μg/ml hDR6-ECD-Fc. The third photograph shows commissural axon degeneration at 48 hours in the presence of 10 μg/ml hDR6-ECD-Fc.

Materials and methods used to generate the data shown in this figure are as follows. Commissural explant cultures and survival assays were prepared and carried out as described above in Examples 2-6. The hDR6-ECD-Fc immunoadhesin protein sequence used in this assay is described above in Example 3. To visualize GFP-labeled commissural axons, pictures were taken on the Axiovert 200 Zeiss inverted microscope (in green fluorescence channel for GFP) using AxioVision40 Release 4.5.0.0 SP1 (March 2006) computer software from Carl Zeiss Imaging Solutions.

As shown in FIG. 11B, hDR6-ECD-Fc delayed sensory axonal degeneration induced by nerve growth factor (NGF) withdrawal. In FIG. 11B from left to right, the upper three photographs show sensory neurons deprived of NGF in the presence of a control Fc at 0, 6 and 24 hours, respectively, while the lower three photographs show sensory neurons deprived of NGF in the presence of the DR6-Fc construct at 0, 6 and 24 hours, respectively.

The disclosure provided in FIG. 11 provides further suggestion that ligand may play an important role for DR6 function in axonal degeneration.

Materials and methods used to generate the data shown in this figure are as follows. To examine whether ligand is required for DR6 function in sensory axonal degeneration, a compartmented culture analysis of sensory axon growth and degeneration was carried out as follows. A Campenot nerve cell chamber system was used to isolate neuronal processes (axons) from the cell bodies in different compartments (separate fluid environments), analogous to neuronal cell bodies in one location of the nervous system projecting their axons to a distal target in another location. The assay was carried out as originally described by Campenot (Campenot et al., J. Neurosci. 11(4): 1126-39 (1991)) with the following modifications. Briefly, 35-mm tissue culture dishes were coated with PDL/Laminin and scratched with a pin rake (Tyler Research) to generate tracks, as illustrated for example in FIGS. 1 and 4 of Campenot et al., supra.

A drop of culture medium (Neurobasal medium with B27 supplement, 25 ng/ml of NGF, and 4 g/L of methylcellulose) was placed on the scratched substratum. A Teflon divider (Tyler Research) was seated on silicone grease and a dab of silicone grease was placed at the mouth of the center slot. Dissociated sensory neurons derived from E12.5 mouse DRGs were suspended in methylcellulose-thickened medium and loaded into a disposable sterile syringe fitted with a 22-gauge needle. This cell suspension was injected into the center slots of each compartmented dish under the dissecting microscope. The neurons were allowed to settle overnight. The outer perimeter of the dish (the cell body compartment) and the inner axonal compartments were filled with methyl-cellulose-containing medium. Within 3-5 days in vitro, axons begin to emerge into the left and right compartments as illustrated for example in FIGS. 1 and 4 of Campenot et al., supra.

To trigger local axonal degeneration, NGF-containing medium from axonal compartments was substituted with neurobasal medium with an NGF blocking antibody (anti-NGF, Genentech, Inc., 20 ug/ml). Zero hours, 6 hours, or 24 to 48 hours following NGF deprivation, sensory neurons were fixed in 4% PFA for 30 minutes at room temperature and processed for immunofluorescence staining with axonal marker TUJ-1 (Covance, 1:500 dilution) to visualize degenerating axons by fluorescence microscopy (FIG. 11B) (as above described in Example 7). To visualize immunofluorescently labeled sensory axons in axonal compartments of the Campenot Chambers, pictures were taken on the Axioplan-2 Imaging Zeiss microscope using AxioVision40 Release 4.5.0.0 SP1 (March 2006) computer software from Carl Zeiss Imaging Solutions.

To examine whether ligand is required for DR6 function in axonal degeneration program triggered by NGF withdrawal, 30 μg/ml of hDR6-ECD-Fc immunoadhesin protein (described in Example 3 above) or 30 μg/ml of a control Fc (Genentech, Inc.) was included together with anti-NGF treatment in axonal compartments of Campenot Chambers. Zero to 24 hours after NGF deprivation, axons in Campenot Chambers were fixed with 4% PFA/PBS and visualized by immuno-fluorescence staining with TUJ-1 (1:500, Covance)/secondary antibody conjugated to a fluorescence group Alexa 488 (Molecular Probes, BD) (FIG. 11B).

NGF deprivation triggered a striking pattern of axonal degeneration, as shown in FIG. 11B. Significantly, addition of hDR6-ECD-Fc immunoadhesin protein delayed the onset of axonal degeneration in this system (FIG. 11B, lower panels). Accordingly, these data suggest soluble ligand may be required for DR6 receptor function in local axonal degeneration induced by removal of growth factors.

### Example 9

### Shedding of DR6 Ligand-Binding Sites from Axons Following NGF Deprivation

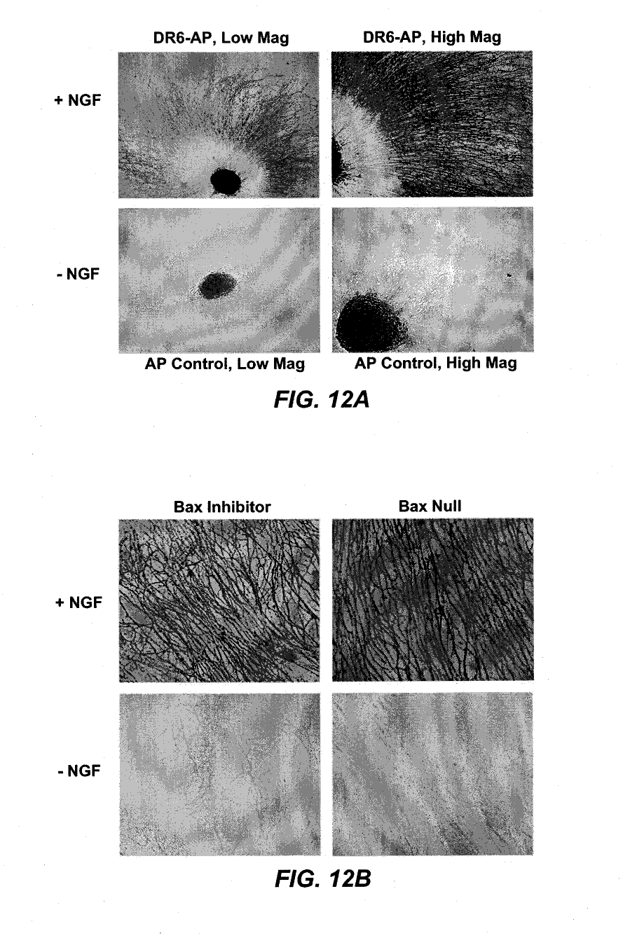

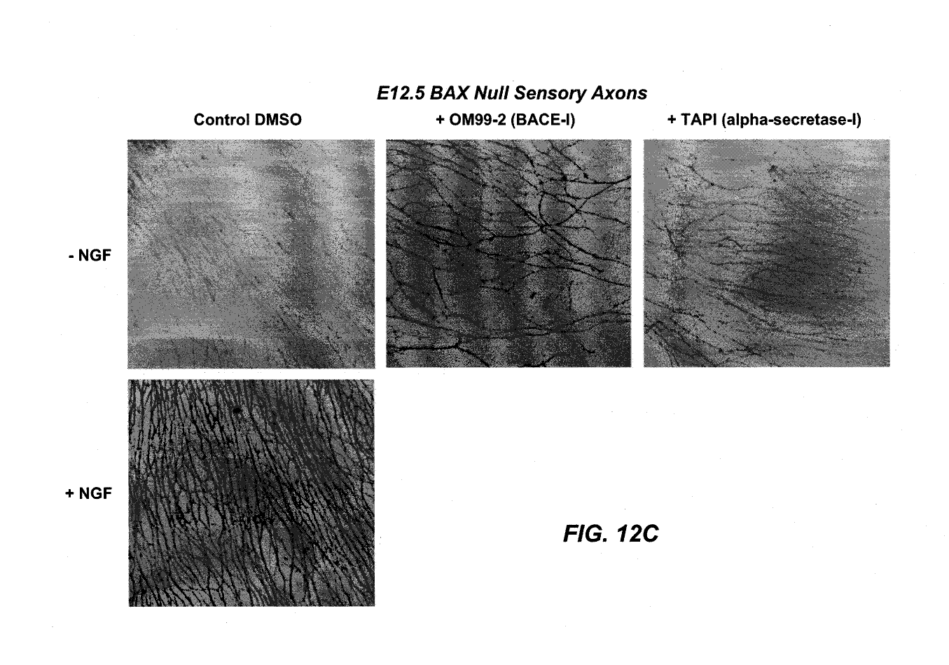

As shown in FIG. 12A and FIG. 12B, a DR6-AP construct was used to visualize DR6 binding sites on sensory axons.

In FIG. 12A from left to right, the upper two photographs show a visualization of DR6 binding sites on sensory axons at developmental stage E12.5 in the presence of NGF at 48 hours using a DR6-AP construct to visualize these axons at low and high magnification respectively, while the lower two photographs show a visualization of sensory axons using a AP control construct at low and high magnification, respectively.

As shown in FIG. 12B, DR6 ligand-binding sites are lost from sensory axons following NGF deprivation.

In FIG. 12B from left to right, the upper two photographs show a visualization of DR6 binding sites on sensory axons, where the first photograph shows sensory neurons in the presence of NGF and a BAX inhibitor while the second photograph shows Bax null sensory neurons in the presence of NGF. The lower two photographs show: sensory neurons in the absence of NGF but in the presence of a BAX inhibitor; and Bax null sensory neurons in the absence of NGF, respectively. Equivalent results are observed in motor axons in the presence and absence of neurotrophins.

The materials and methods used to generate the data shown in FIG. 12A and FIG. 12B are as follows. The DR6-AP construct was generated by fusing a mouse DR6 ectodomain to human placental alkaline phosphatase (DR6-AP), using pRK5-AP cloning vector (see, e.g. Yan et al., Nature Immunology 1, 37-41 (2000)). The PRK5 parental cloning vector is available from the Becton, Dickinson and Company, Pharmingen division. The murine DR6 ectodomain sequence used to generate the DR6-AP fusion protein is as follows:

(SEQ ID NO: 14)
MGTRASSITALASCSRTAGQVGATMVAGSLLLLGFLSTITAQPEQKT
LSLPGTYRHVDRTTGQVLTCDKCPAGTYVSEHCTNMSLRVCSSCPAG
TFTRHENGIERCHDCSQPCPWPMIERLPCAALTDRECICPPGMYQSN
GTCAPHTVCPVGWGVRKKGTENEDVRCKQCARGTFSDVPSSVMKCKA
HTDCLGQNLEVVKPGTKETDNVCGMRLFFSSTNPPSSGTVTFSHPEH
MESHDVPSSTYEPQGMNSTDSNSTASVRTKVPSGIEEGTVPDNTSST
SGKEGTNRTLPNPPQVTHQQAPHHRHILKLLPSSMEATGEKSSTAIK
APKRGHPRQNAHKHFDINEH

The Bax null mouse line (Bax-R1) has been described previously (Deckwerth et al., Neuron, 17:401-411, 1996) and was obtained from Jackson Laboratories. The BAX inhibitory peptide was used at 10 uM to block neuronal cell death (Bax-V5, Tocris Inc).

To generate mouse DR6 ectodomain-AP fusion protein (DR bv6-AP), COS-1 cells cultured in DMEM/10% FBS (Gibco) medium were transfected with 15 microgram of DR6-AP fusion expression construct using FuGene transfection reagent (Roche) according to manufacturer protocol. Twelve hours post-transfection, COS-1 cell medium was changed to OPTI-MEM (Invitrogen). Forty-eight hours post-transfection, COS-1 cell conditioned medium containing DR6-AP proteins was collected and filtered. The amount of DR6-AP proteins in the medium was quantified as follows:

100 microliter of 2×AP buffer (prepared by adding 100 mg Para-nitrophenyl phosphate (Sigma) and 15 microliter of 1M MgCl₂ to 15 ml 2M diethanolamine pH 9.8) was mixed with equal volume of transfected COS cell conditioned medium or control conditioned medium from untransfected COS-1 cells. The color of the reaction was developed over 12-15 minutes, with the O.D. being in the linear range (0.1-1). The volume of reaction was than adjusted by adding 800 microliter of distilled water and the O.D. was measured at 405 nm absorbance wavelength. The concentration in nM was calculated according to the formula (for 100 microliter): C (nM)=O.D.×100×(60/developing time)/30.

For the in situ DR6-AP sensory axon binding assay, either wild-type or Bax null sensory explants were cultured in Neurobasal medium/B27 (Invitrogen) as outlined in the Examples 7-8 above, with 50 ng/ml NGF (Roche). Two days post-plating, DRG explants were either left untreated or deprived from NGF as described above in Examples 7-8. Bax inhibitory peptide was added where indicated on FIG. 12B (10 uM, Bax-V5, Tocris). Twelve hours post-NGF deprivation, DRG explants were washed twice with the binding buffer (HBSS, Gibco Cat. No. 14175-095, with 0.2% BSA, 0.1% NaN₃, 5 mM CaCl₂, 1 mM MgCl₂, 20 mM HEPES, pH=7.0). AP binding assay was then carried out by making a 1:1 mixture of DR6-AP conditioned medium and the binding buffer (or control AP conditioned medium and the binding buffer), which was applied directly to DRG explants in 8-well culture slides (Becton, Dickinson and Company) and incubated for 90 minutes at room temperature.

Following the incubation, unbound DR6-AP proteins were washed away by rinsing DRG explants five times with the binding buffer. DRG explants were then fixed with 3.7% formaldehyde diluted in PBS, for 12 minutes at room temperature. The remaining formaldehyde was removed by rinsing DRG explants 3 times with HBS buffer (20 mM HEPES pH=7.0, 150 mM NaCl). Endogenous AP activity was blocked by heat inactivation at 65° C. in HBS buffer for 30 minutes. DRG explants were then rinsed three times in the AP reaction buffer (100 mM TRIS pH=9.5, 100 mM NaCl, 50 mM MgCl₂). DR6-AP fusion protein binding to sensory axons was then visualized by developing color stain on DRG explants in AP reaction buffer with 1/50 (by volume) of NBT/BCIP stock solution (Roche, Cat. No. 1681451), overnight at room temperature (FIG. 12A and FIG. 12B). In a parallel control experiment, conditioned medium from AP-transfected COS cells was used for the AP axon binding assay (FIG. 12A, lower panels).

As seen in FIG. 12B, DR6-AP binding sites are lost from sensory axon surface following NGF deprivation, suggesting DR6 ligand is released into axon conditioned medium after trophic deprivation.

As shown in FIG. 12C, studies of BAX null sensory axons at developmental stages E12.5 show that a Beta secretase (BACE) inhibitor can block the disappearance of DR6-AP binding sites from sensory axons following NGF withdrawal. In FIG. 12C from left to right, the upper three photographs show these neurons in the presence of: a DMSO control; OM99-2 (BACE-I inhibitor) and TAP1 (alpha secretase-I inhibitor), respectively. The lower photograph shows these neurons in the presence of NGF.

The mouse DR6 ectodomain-AP fusion protein used to generate this data is described above. The Bax null mouse line (Bax-R1) have been described previously (Deckwerth et al., Neuron, Vol. 17, 401-411, 1996) and has been obtained from Jackson Laboratories. DRG explant cultures and DR6-AP axon binding assay were carried out as described above for FIG. 12A and FIG. 12B. The BACE inhibitor was used in the assay at 1 uM final concentration (InSolution OM99-2, Calbiochem/Merck). The alpha-secretase inhibitor TAPI was used in the assay at 10 uM final concentration (TAPI-1, Calbiochem). To visualize DR6-AP-positive sensory axons (stained by AP colorimetric stain reaction outlined in the Example 9 above), bright field pictures were taken on the Axioplan-2 Imaging Zeiss microscope using AxioVision40 Release 4.5.0.0 SP1 (March 2006) computer software from Carl Zeiss Imaging Solutions.

### Example 10

### Amyloid Precursor Protein (APP) is a Cognate Ligand of DR6

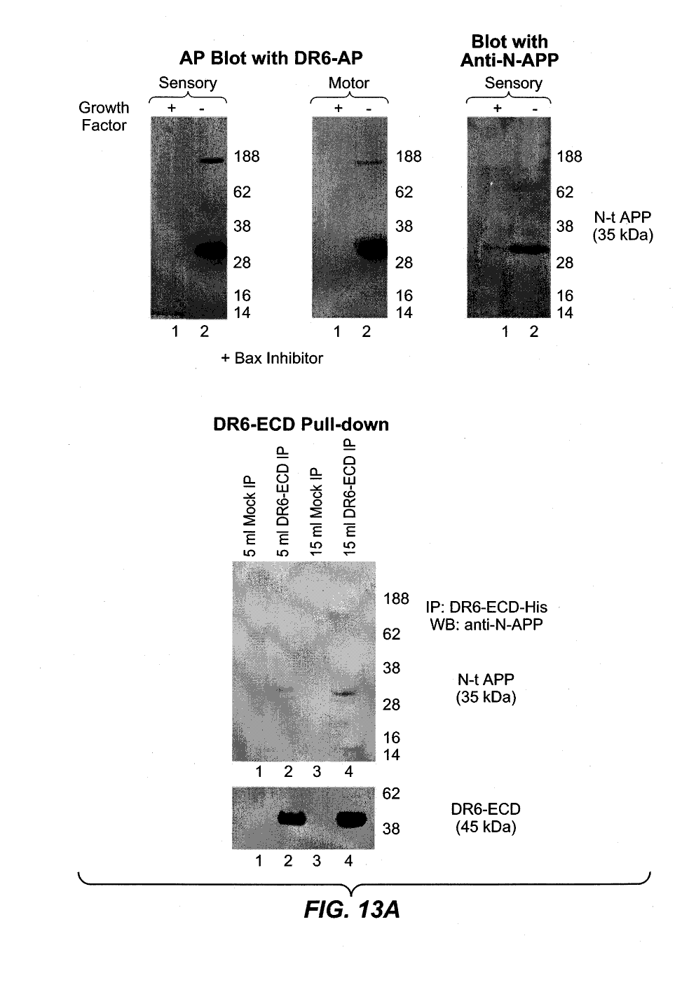

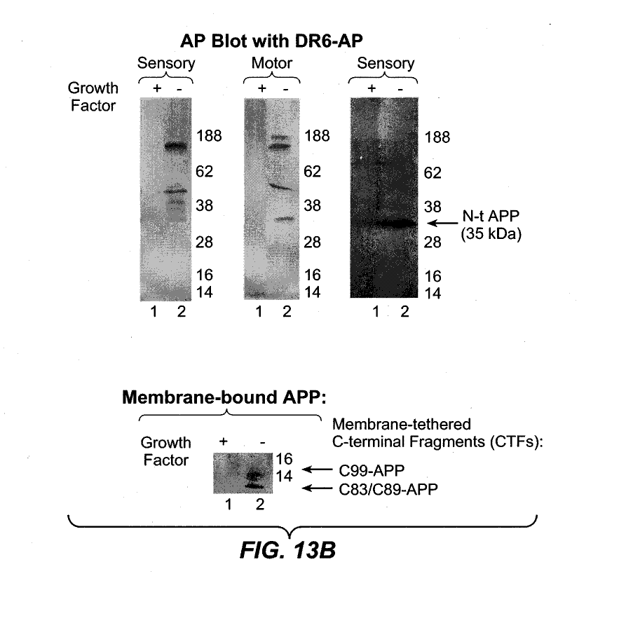

As shown in FIG. 13, N-APP was found to be a DR6 ectodomain-associated ligand.

In FIG. 13A from left to right, the first two blots provide data from studies using a DR6-AP construct to probe proteins obtained from sensory and motor neurons in the presence and absence of growth factor (and in the presence of a Bax inhibitor). In these blots, APP polypeptides including a strong band at approximately 35 kDA are observed in both sensory and motor neurons deprived of growth factor (and in the presence of a Bax inhibitor). The central blot in FIG. 13A shows that APP polypeptides including the strong band at approximately 35 kDA are correspondingly observed with anti-N-APP antibody probe of polypeptides obtained from sensory neurons deprived of growth factor. The polyclonal anti-N-APP antibody used for the Western blot experiments at 1:100 dilution was obtained from Thermo Scientific (Cat. No. RB-9023-P1). The Bax inhibitor peptide P5 was used at 10 μM (Tocris Biosciences, Cat. No. 1786, cell-permeable synthetic peptide inhibitor of Bax translocation to mitochondria).

The observation that APP is a DR6 ectodomain-associated ligand was further confirmed by data presented in the blot shown in the right of FIG. 13A. A general pull-down protocol (e.g., Nikolaev et al., 2004, BBRC, 323, 1216-1222) was used to purify DR6-ECD ectodomain associated factors from sensory axon conditioned medium that was collected from axonal compartments of Campenot Chambers under conditions of NGF deprivation. DR6-ECD-His ectodomain (construct described below)-coupled NiNTA beads (Sigma) were incubated with 50 ml of sensory axon conditioned medium under the following conditions: 150 mM NaCl, 0.2% NP-40 (Calbiochem), 1×PBS buffer, for overnight at 4° C. DR6-ECD-His ectodomain-coupled NiNTA beads (Sigma) were then washed 5 times with 10-fold excess of the binding buffer (150 mM NaCl, 0.2% NP-40 (Calbiochem), in 1×PBS buffer), and DR6-ECD-associated protein complexes were eluted out with 1×SDS sample loading buffer (Invitrogen)) which were then separated via gel electrophoresis and probed with anti-N-APP antibody. The data from this DR6-ECD pull down experiment correspondingly identifies APP polypeptides including a strong band at approximately 35 kDA.

The DR6-AP blot assay on axon conditioned medium was carried out according to the protocol described previously (Pettmann et al., 1988, J. Neurosci. 8(10):3624-3632). The polyclonal anti-N-APP antibody used for Western blot experiments was obtained from Thermo Scientific (Cat. No. RB-9023-P1). The mouse DR6 ectodomain-AP fusion protein used was described above in Example 9. Mouse recombinant DR6-ECD-His was expressed and subsequently purified from CHO cell cultures. The amino acid sequence of the murine DR6-ECD-His is as follows:

(SEQ ID NO: 15)
MGTRASSITALASCSRTAGQVGATMVAGSLLLLGFLSTITAQPEQKT
LSLPGTYRHVDRTTGQVLTCDKCPAGTYVSEHCTNMSLRVCSSCPAG
TFTRHENGIERCHDCSQPCPWPMIERLPCAALTDRECICPPGMYQSN
GTCAPHTVCPVGWGVRKKGTENEDVRCKQCARGTFSDVPSSVMKCKA
HTDCLGQNLEVVKPGTKETDNVCGMRLFFSSTNPPSSGTVTFSHPEH
MESHDVPSSTYEPQGMNSTDSNSTASVRTKVPSGIEEGTVPDNTSST
SGKEGTNRTLPNPPQVTHQQAPHHRHILKLLPSSMEATGEKSSTAIK
APKRGHPRQNAHKHFDINEHHHHHH

FIG. 13B shows another visualization of DR6 ligand in axon conditioned media by DR6-AP blotting. This blotting data identifies a number of APP polypeptides including the N-terminal APP at 35 kDa as well as the C99-APP and C83/C89 APP polypeptides. The DR6-AP blot assay on axon conditioned medium was carried out according to the protocol described previously (Pettmann et al., 1988, J. Neurosci. 8(10): 3624-3632). The mouse DR6 ectodomain-AP fusion protein was generated as described above in Example 8. Mouse recombinant DR6-ECD-His was expressed and subsequently purified from CHO cell cultures. The amino acid sequence of DR6-ECD-His is shown above. The polyclonal anti-N-APP antibody used for Western blot experiments was obtained from Thermo Scientific (Cat. No. RB-9023-P1). To visualize Membrane-tethered APP C-terminal fragments (CTFs) C99-APP and C83/C89-APP, Western Blot analysis of axonal lysates was carried out using 4G8 antibody that recognizes an epitope within the central part of Abeta (monoclonal 4G8, 1:500, Covance).

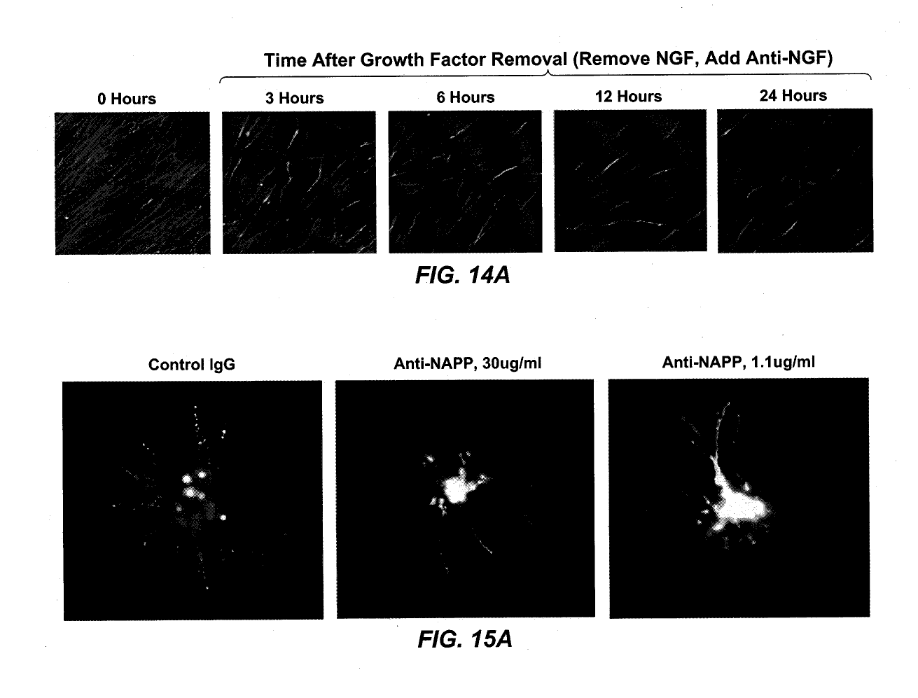

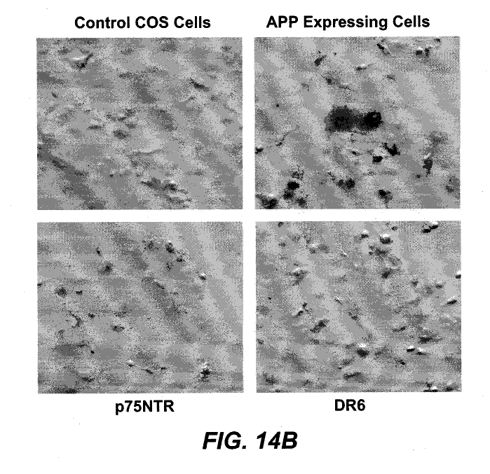

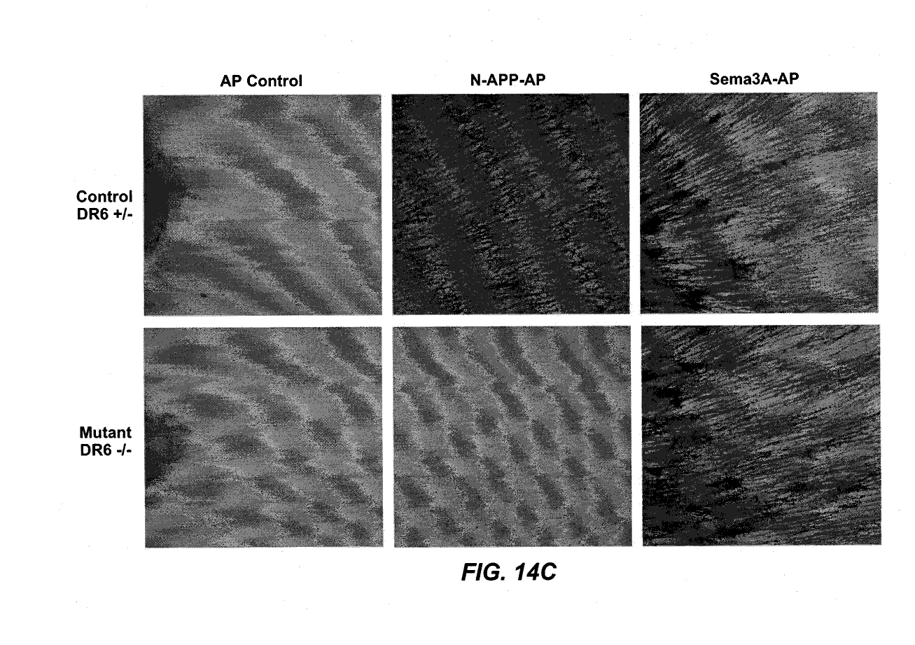

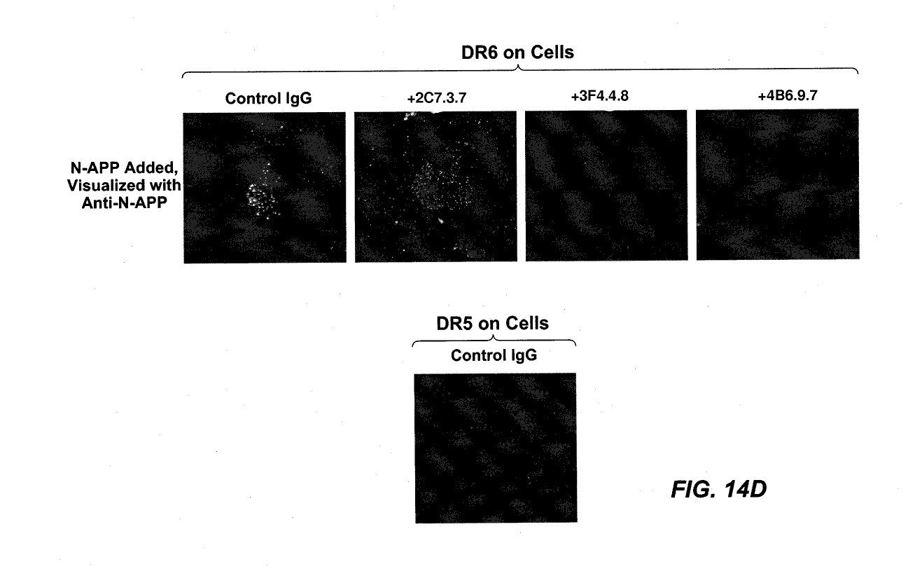

FIG. 14A provides photographs showing that shedding of the APP ectodomain occurs early on after NGF deprivation. In FIG. 14A, neurons at various times post growth factor removal were stained with a N-APP polyclonal antibody in the presence of a Bax inhibitor added to block axonal degeneration. From left to right, these photographs show axonal degeneration at 0 hours as well as 3, 6, 12 and 24 hours after the removal of NGF (and the addition of anti-NGF antibodies).

The polyclonal anti-N-APP antibody used to visualize surface APP expression in APP axon shedding experiments was obtained from Thermo Scientific (Cat. No. RB-9023-P1). The sensory explant cultures were carried out as described in Example 6 and 7 above. NGF deprivation assay was carried out as described above in Example 7 with the modifications as follows. DRG explant cultures were fixed in 4% PFA/PBS after indicated time intervals following NGF deprivation: 0 hours, 3 hours, 6 hours, 12 hours, and 24 hours. To visualize surface APP expression, DRG axons were processed for immunofluorescence stain as in Examples 6 and 7, without the Triton permeabilization step, using the above described anti-N-APP primary antibody.

To visualize surface APP expression on sensory axons (immunofluorescently labeled with anti-N-APP antibody, Thermo Scientific (Cat. No. RB-9023-P1)), pictures were taken on the Axioplan-2 Imaging Zeiss microscope (in red fluorescence channel) using AxioVision40 Release 4.5.0.0 SP1 (March 2006) computer software from Carl Zeiss Imaging Solutions.

FIG. 14B provides photographs showing that the DR6 ectodomain binds APP expressed by cultured cells. In FIG. 14B from left to right, the upper two photographs show control COS cells and APP expressing cells, respectively probed, with DR6-APP (having the DR6 ectodomain). The lower two photographs show p75NTR receptor and DR6 receptor expressing cells probed with DR6-AP. DR6 ectodomain does NOT bind to p75NTR or to DR6 receptor expressing cells.

The materials and methods used to generate the data shown in this figure are as follows. To test whether APP directly interacts with DR6 extracellular domain, a cell-based A binding assay was carried out (FIG. 14B). To generate DR6 ectodomain-A fusion protein (DR6-AP), COS-1 cells cultured in DMEM/10% FBS (Gibco) medium were transfected with 15 microgram of DR6-AP fusion expression construct using FuGene transfection reagent (Roche) according to the manufacturer protocol. Twelve hours post-transfection, COS-1 cell medium was changed to OPTI-MEM (Invitrogen). Forty-eight hours post-transfection, COS-1 cell conditioned medium containing DR6-AP proteins was collected and filtered.

The amount of DR6-AP proteins in the medium was quantified according to the following procedure. 100 microliters of 2×AP buffer (prepared by adding 100 mg Para-nitrophenyl phosphate (Sigma) and 15 microliter of 1M MgCl₂ to 15 ml 2M diethanolamine pH 9.8) was mixed with equal volume of transfected COS cell conditioned medium or control conditioned medium from untransfected COS-1 cells. The color of the reaction was developed over 12-15 minutes, with the O.D. being in the linear range (0.1-1). The volume of reaction was then adjusted by adding 800 microliters of distilled water and the O.D. was measured at 405 nm absorbance wavelength. The concentration in nM was calculated according to the formula (for 100 microliter): C (nM)=O.D.×100×(60/developing time)/30.

For the APP AP binding assay, COS-1 cells cultured in DMEM/10% FBS (Gibco) medium in 6-well culture dishes were transfected with 2 microgram of APP expressing vector per well using FuGene transfection reagent (Roche) according to the manufacturer protocol. Two days post-transfection, cells were washed twice with the binding buffer (HBSS, Gibco Cat. No. 14175-095, with 0.2% BSA, 0.1% NaN₃, 5 mM CaCl₂, 1 mM MgCl₂, 20 mM HEPES, pH=7.0). An AP binding assay was then carried out by making a 1:1 mixture of DR6-AP conditioned medium and the binding buffer, which was applied directly to APP over-expressing COS-1 cells and incubated for 90 minutes at room temperature. Following the incubation, the unbound DR6-AP proteins were washed away by rinsing COS-1 cells five times with the binding buffer. Cells were then fixed with 3.7% formaldehyde diluted in PBS, for 12 minutes at room temperature. The remaining formaldehyde was removed by rinsing cells 3 times with HBS buffer (20 mM HEPES pH=7.0, 150 mM NaCl). Endogenous AP activity was blocked by heat inactivation at 65° C. in HBS buffer for 30 minutes. COS-1 cells were then rinsed three times in the AP reaction buffer (100 mM TRIS pH=9.5, 100 mM NaCl, 50 mM MgCl₂). DR6-AP fusion protein binding to transmembrane APP was then visualized by developing color reaction on COS-1 cells in AP binding buffer with 1/50 (by volume) of NBT/BCIP stock solution (Roche, Cat. No. 1681451), for overnight at room temperature (FIG. 14B). In a parallel control experiment, conditioned medium from untransfected COS cells was used for the AP binding assay. Transmembrane p75NTR and DR6 receptors expressed in COS-1 cells showed no specific binding to DR6-AP fusion protein (FIG. 14B) under the same experimental conditions, indicating that the interaction between DR6 ectodomain and APP is specific.

FIG. 14C provides photographs showing that DR6 is the major receptor for N-APP on sensory axons and that APP binding sites are significantly depleted in the neuronal cells of DR6 null mice. In FIG. 14C from left to right, the upper three photographs show neurons obtained from a DR6+/− (het) mouse probed with an AP control, N-APP-AP, and Sema3A-AP, respectively. The lower three photographs correspondingly show neurons obtained from a DR6−/− (KO) mouse probed with an AP control, N-APP-AP, and Sema3A-AP, respectively.

The materials and methods used to generate the data shown in FIG. 14C are as follows. The mouse DR6 ectodomain-AP fusion protein was generated as described above in Example 9 above. The mouse Sema3A ectodomain-AP (Sema3A-AP) fusion protein was generated as described previously (Feiner et al., 1997, Neuron 19:539-545). The DR6 null mouse line (DR6.KO) has been described previously (Zhao et al., J. Exp. Med. 194:1441-1441, 2001). DRG explant cultures and DR6-AP axon binding assay were carried out as described above in Example 9 for FIG. 12A and FIG. 12B.

FIG. 14D provides photographs showing that antagonist DR6 antibodies disrupted the interaction between the DR6 ectodomain and neuronal APP. In these studies, N-APP was added to neuronal cells expressing DR6 and then visualized with anti-N-APP antibody. From left to right, the first four photographs show the ability of N-APP to bind DR6 on the surface of neurons in the presence of: a control IgG; the 2C7.3.7 anti-DR6 antibody; the 3F4.4.8 anti-DR6 antibody; and the 4B6.9.7 anti-DR6 antibody, respectively. The photograph on the far right shows staining of DR6 on cells using a control IgG.

The materials and methods used to generate the data shown in this figure are as follows. The cell-based ligand binding assay used to obtain the data shown in FIG. 14D was carried out as described previously (Okada et al., Nature 2006, 444:369-373), with the following modifications. To generate N-terminal growth factor-like domain APP-His fusion protein (N-APP-His), COS-1 cells cultured in DMEM/10% FBS (Gibco) medium were transfected with 15 microgram of N-APP-His fusion expression construct using FuGene transfection reagent (Roche) according to the manufacturer protocol. Twelve hours post-transfection, COS-1 cell medium was changed to OPTI-MEM (Invitrogen). Forty-eight hours post-transfection, COS-1 cell conditioned medium containing N-APP-His proteins was collected and filtered. The concentration of N-APP-His was determined by western blot analysis with above described anti-N-APP antibody.

The amino acid sequence of human N-APP-His used in this binding assay is as follows:

(SEQ ID NO: 10)
MLPGLALLLLAAWTARALEVPTDGNAGLLAEPQIAMFCGRLNMHMNV
QNGKWDSDPSGTKTCIDTKEGILQYCQEVYPELQITNVVEANQPVTI
QNWCKRGRKQCKTHPHFVIPYRCLVGEFVSDALLVPDKCKFLHQERM
DVCETHLHWHTVAKETCSEKSTNLHDYGMLLPCGIDKFRGVEFVCCP
LAEESDNVDSADAEEDHHHHHH

The N-APP-His binding assay was then carried out by making a 1:1 mixture of N-APP-His conditioned medium and the binding buffer, which was applied directly to DR6 receptor over-expressing COS-1 cells and incubated for 90 minutes at room temperature. Where indicated, DR6 mAbs 4B6.9.7, 3F4.4.8 or 2C7.3.7 (above described, Examples 3 and 7) were added individually at 20 ug/ml together with N-APP-His conditioned medium and the binding buffer. Normal mouse IgG (Genentech Inc) was added at 20 ug/ml together with N-APP-His conditioned medium and the binding buffer in a control experiment.

N-APP binding to DR6 receptor expressing cells was visualized by immunofluorescence stain with the anti-N-APP antibody (Thermo Scientific Cat. No. RB-9023-P1) according to known protocols as described in protocols of Examples 6 and 7 (Okada et al., Nature, 2006, Vol. 444, 369-373). To visualize N-APP protein bound to DR6 receptor on cell surface (immunofluorescently labeled with anti-N-APP antibody, Thermo Scientific (Cat. No. RB-9023-P1)), pictures were taken on the Axioplan-2 Imaging Zeiss microscope (in red fluorescence channel) using AxioVision40 Release 4.5.0.0 SP1 (March 2006) computer software from Carl Zeiss Imaging Solutions.

### Example 11

### Amyloid Precursor Protein (APP) Activates DR6 to Induce Axonal Degeneration

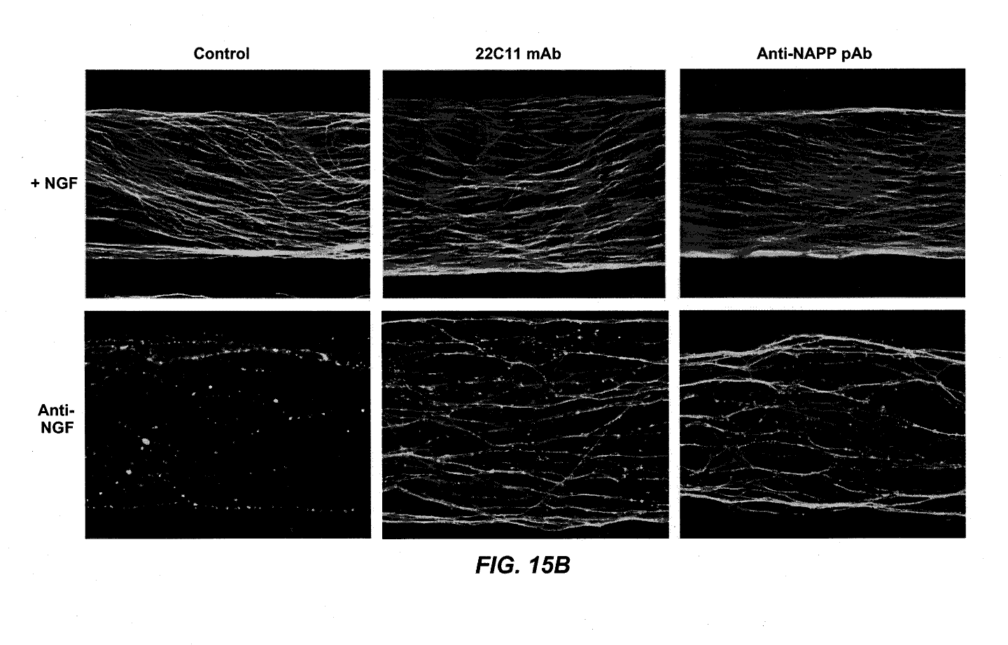

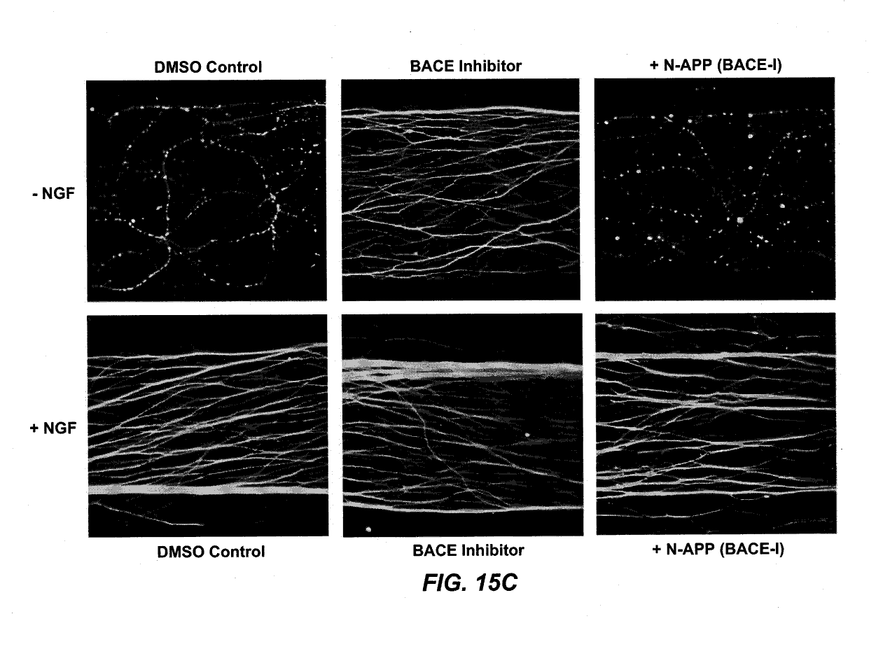

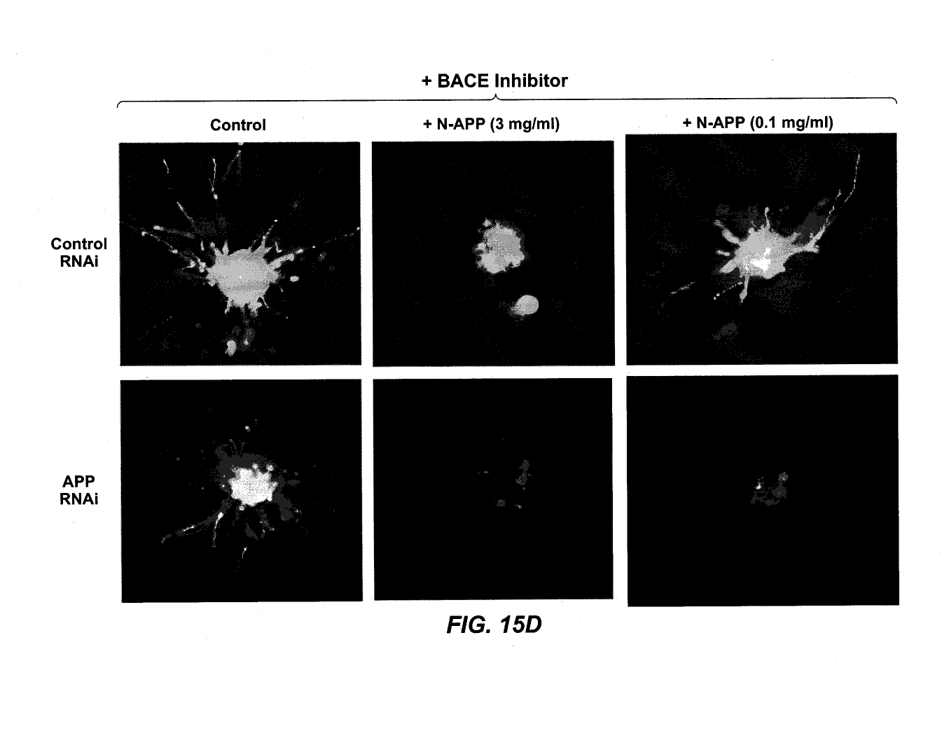

FIG. 15A provides photographs showing polyclonal antibody to N-terminal APP blocks axonal degeneration in a commissural axon assay. From left to right, the photographs in FIG. 15A show commissural axon degeneration in the presence of: a control IgG; 30 μg/ml of an anti-NAPP antibody; and 1.1 μg/ml of an anti-NAPP antibody, respectively.

The materials and methods used to generate the data shown in FIG. 15A are as follows. The commissural explant survival assay was carried out with indicated quantities of the polyclonal anti-N-APP antibody (Thermo Scientific Cat. No. RB-9023-P1, extensively dialyzed) or control IgG (rabbit IgG, R&D systems) as described in protocols of Example 2 and the data generated in FIG. 4B. To visualize GFP-labeled commissural axons, pictures were taken on the Axiovert 200 Zeiss inverted microscope (in green fluorescence channel for GFP) using AxioVision40 Release 4.5.0.0 SP1 (March 2006) computer software from Carl Zeiss Imaging Solutions.

FIG. 15B provides photographs showing that N-terminal APP antibodies inhibited sensory axonal degeneration induced by NGF removal. From left to right, the upper three photographs of FIG. 15B show sensory axons in the presence of NGF and: a control antibody; anti-APP monoclonal antibody 22C11; and anti-APP polyclonal antibodies, respectively. The lower three photographs correspondingly show sensory axons in the absence of NGF (as well as an anti-NGF antibody) and: a control antibody; anti-APP monoclonal antibody 22C11; and anti-APP polyclonal antibodies, respectively.

The materials and methods used to generate the data shown in this FIG. 15B are as follows. The NGF deprivation assay was carried out in Campenot Chambers as described above in Example 8. Antibodies to N-terminal APP used in the assay were polyclonal anti-N-APP antibody (Thermo Scientific Cat. No. RB-9023-P1, extensively dialyzed) or 22C11 monoclonal antibody (22C11, Chemicon, extensively dialyzed). Normal IgG (rabbit IgG, R&D systems) was added as a control experiment Immunofluorescence labeling of sensory axons with TUJ1 antibody (1:500, Covance) was carried out as described in Examples 1, 7 and 8. To visualize immunofluorescently labeled sensory axons in axonal compartments of the Campenot Chambers, pictures were taken on the Axioplan-2 Imaging Zeiss microscope using AxioVision40 Release 4.5.0.0 SP1 (March 2006) computer software from Carl Zeiss Imaging Solutions.

FIG. 15C provides photographs showing that axonal degeneration that is blocked by inhibition of β-secretase (BACE) activity can be rescued by the addition of N-APP. From left to right, the upper three photographs in FIG. 15C show neurons (cultured in the absence of NGF) and the axonal degeneration observed in the presence of: a DMSO control, a BACE inhibitor, and N-APP (and BACE-I) respectively. The lower three photographs in FIG. 15C correspondingly show neurons (cultured in the presence of NGF) as well as: a DMSO control, a BACE inhibitor, and N-APP (and BACE-I) respectively.

Materials and methods used to generate the data shown in this FIG. 15C are as follows. The NGF deprivation assay was carried out in Campenot Chambers as described above in Example 8. The human recombinant N-APP amino acids 19-306 used in this assay was purchased from Novus (Novus Biologicals, Cat. No. H00000351-P01). N-APP was added at 3 μg/ml together with BACE inhibitor (1 uM final concentration, InSolution 0M99-2, Calbiochem/Merck), at the time of NGF deprivation. The BACE inhibitor was used in the assay at 1 uM final concentration (InSolution 0M99-2, Calbiochem/Merck). Immunofluorescence labeling of sensory axons with TUJ1 antibody (1:500, Covance) was carried out as described in Examples 1, 7 and 8. To visualize immunofluorescently labeled sensory axons in axonal compartments of the Campenot Chambers, pictures were taken on the Axioplan-2 Imaging Zeiss microscope using AxioVision40 Release 4.5.0.0 SP1 (March 2006) computer software from Carl Zeiss Imaging Solutions.

FIG. 15D provides photographs showing APP removal by RNAi sensitizes neuronal cells grown in the presence of BACE inhibitor to cell death induced by N-APP. In FIG. 15D from left to right, the upper three photographs show neurons cultured in the presence of a control RNAi. These upper photographs show a control as well as neurons cultured with 3 μg/ml N-APP or 0.1 μg/ml N-APP respectively. The lower three photographs show neurons cultured in the presence of an APP RNAi. These lower photographs show a control as well as neurons cultured with 3 μg/ml N-APP or 0.1 μg/ml N-APP respectively.

Materials and methods used to generate the data shown in this FIG. 15D are as follows. The APP RNAi in commissural explant cultures was carried out as described in Example 2. The human recombinant N-APP amino acids 19-306 used in this assay was purchased from Novus (Novus Biologicals, Cat. No. H00000351-P01). Pre-designed rat-specific APP ON-TARGETplus siRNA pool was used in this assay according to manufacturer protocols to down-regulate APP expression in E13 rat commissural explants (APP ON-TARGETplus siRNA pool, GeneID: 54226, Cat. No. 088191, Dharmacon Inc.). To visualize GFP-labeled and RFP-labeled commissural axons (as described in Examples 2 and 7), pictures were taken on the Axiovert 200 Zeiss inverted microscope (in green fluorescence channel for GFP) using AxioVision40 Release 4.5.0.0 SP1 (March 2006) computer software from Carl Zeiss Imaging Solutions.

### Example 12

### DR6 is Required for APP Induced Axonal Degeneration but not Degeneration Triggered by Abeta

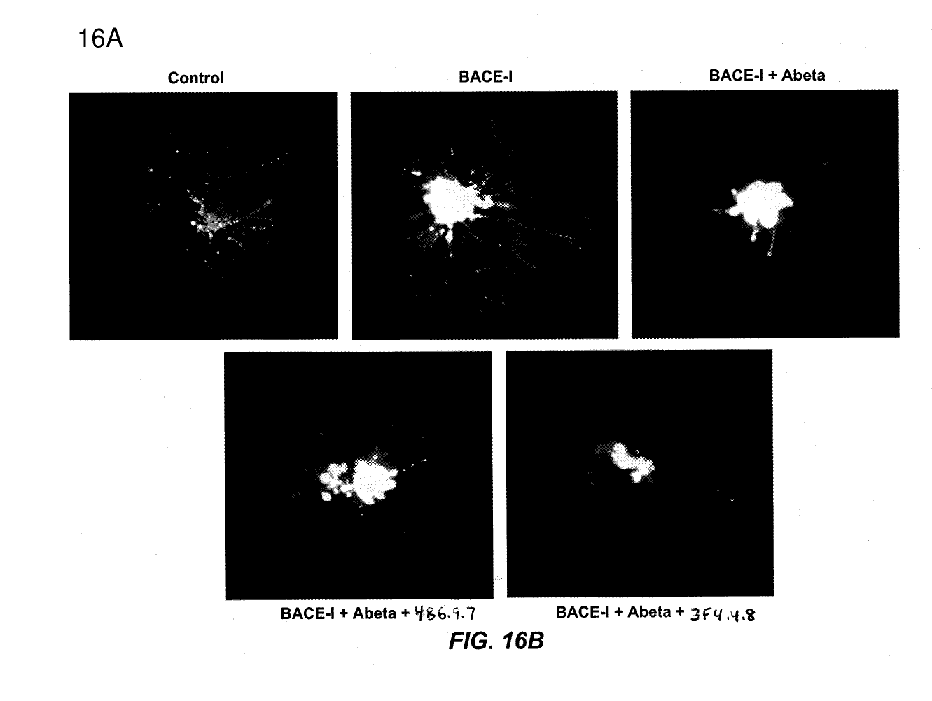

As shown in FIG. 16A, DR6 activation is required for N-APP induced axonal degeneration.

In FIG. 16A from left to right, the upper three photographs show neurons obtained from a DR6+/−(het) mouse. The first photograph shows control neurons not exposed to Abeta or N-APP, the second photograph shows neurons exposed to Abeta, and the third photograph shows neurons exposed to N-APP. The lower three photographs show neurons obtained from a DR6−/− (KO) mouse. From left to right, the lower first photograph shows control neurons not exposed to Abeta or N-APP, the second photograph shows neurons exposed to Abeta, and the third photograph shows neurons exposed to N-APP.

Materials and methods used to generate the data shown in this FIG. 16A are as follows. Commissural explant cultures and survival assay were carried out as described in Example 2. The DR6 null mouse line (DR6.KO) has been described previously (Zhao et al., J. Exp. Med. 194:1441-1441, 2001). The human recombinant N-APP amino acids 19-306 used in this assay was purchased from Novus (Novus Biologicals, Cat. No. H00000351-P01). The recombinant human Beta amyloid amino acids 1-42 used in this assay was purchased from Chemicon (ultra pure human Abeta 1-42, Cat. No. AG912, Chemicon). N-APP was added to commissural explants at 3 μg/ml, 24 hours after plating, together with the BACE inhibitor. The recombinant human Beta amyloid amino acids 1-42 was added to commissural explants at 3 μM, 24 hours after plating, together with the BACE inhibitor. The BACE inhibitor was used in the assay at 1 uM final concentration (InSolution 0M99-2, Calbiochem/Merck). Commissural explants were incubated with indicated amounts of N-APP or Abeta for additional 24 hours. Data was collected 48 hours after commissural explant plating. To visualize commissural axons, pictures were taken on the Axiovert 200 Zeiss inverted microscope (in the bright field) using AxioVision40 Release 4.5.0.0 SP1 (March 2006) computer software from Carl Zeiss Imaging Solutions.

As shown in FIG. 16B, the antagonist DR6 antibodies failed to block axonal degeneration triggered by Abeta. In FIG. 16B from left to right, the upper three photographs show control neurons, neurons in the presence of BACE-I and neurons in the presence of BACE-I and Abeta. In FIG. 16B, the lower two photographs show neurons in the presence of BACE-I, Abeta and anti-DR6 antibody 4B6.9.7, and then neurons in the presence of BACE-I, Abeta and anti-DR6 antibody 3F4.4.8.

Materials and methods used to generate the data shown in this FIG. 16B are as follows. Commissural explant cultures and survival assay were carried out as described in Example 2. The recombinant human Beta amyloid amino acids 1-42 used in this assay was purchased from Chemicon (ultra pure human Abeta 1-42, Cat. No. AG912, Chemicon). The BACE inhibitor was used in the assay at 1 uM final concentration (InSolution 0M99-2, Calbiochem/Merck). The recombinant human Beta amyloid amino acids 1-42 was added to commissural explants at 3 μM, 24 hours after plating, together with the BACE inhibitor and indicated anti-DR6 mAbs at 40 ug/ml. The BACE inhibitor was used in the assay at 1 uM final concentration (InSolution 0M99-2, Calbiochem/Merck). Commissural explants were incubated with indicated amounts of Abeta for an additional 24 hours. Data was collected 48 hours after commissural explant plating.

The mouse monoclonal 4B6.9.7, IE5.5.7, 3F4.4.8, 2C7.3.7 and 3B11.7.7 DR6 antibodies were generated by immunizing a mouse with DR6 ectodomain as described in Example 3 above. As noted above, the DR6 antibodies designated here as 4B6.9.7 and 3F4.4.8 antibodies are the DR6 antibodies described in Example 3. To visualize GFP-labeled commissural axons, pictures were taken on the Axiovert 200 Zeiss inverted microscope (in the green fluorescence channel for GFP) using AxioVision40 Release 4.5.0.0 SP1 (March 2006) computer software from Carl Zeiss Imaging Solutions.

### Example 13

### Intracellular DR6 Signaling

Caspases are importants factors in the programmed cell death pathway (see, e.g. Gruffer et al., Curr. Opin. Struct. Biol. 10(6):649-55 (2000); Kuida et al., Nature 384(6607):368-72 (1996): and Finn et al., J. Neurosci. 20(4):1333-41 (2000)), and some caspases are associated with intracellular signaling in neurodegenerative diseases including Huntington's disease and AD (see, e.g. Wellington et al., J. Neurosci. 22(18):7862-72 (2002); Graham et al., Cell 125(6):1179-91 (2006); Guo et al., Am. J. Pathol. (2):523-31 (2004); and Horowitz et al., J. Neurosci. 24(36):7895-902 (2004)).

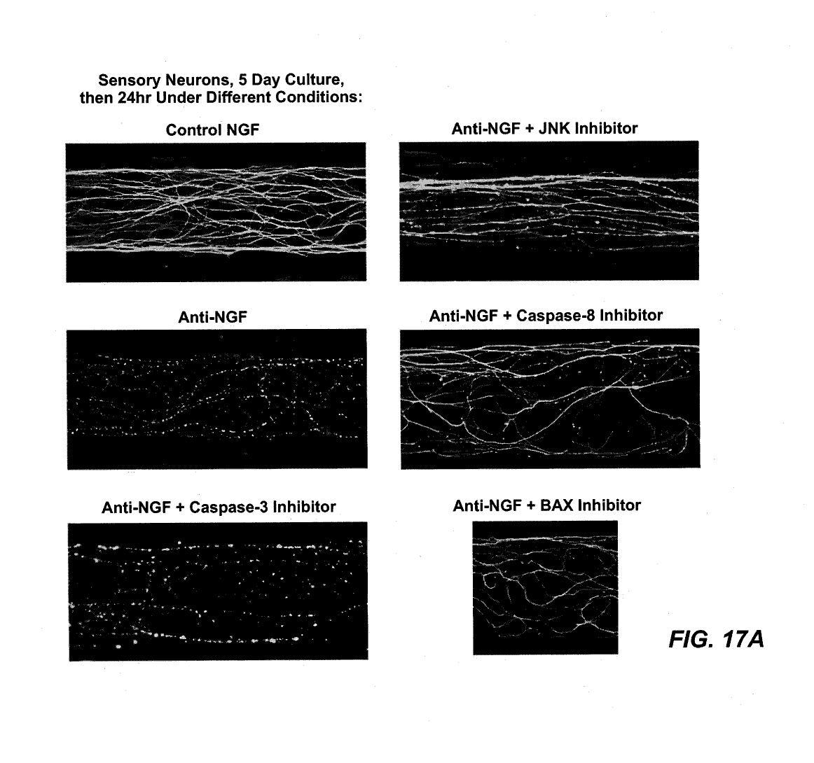

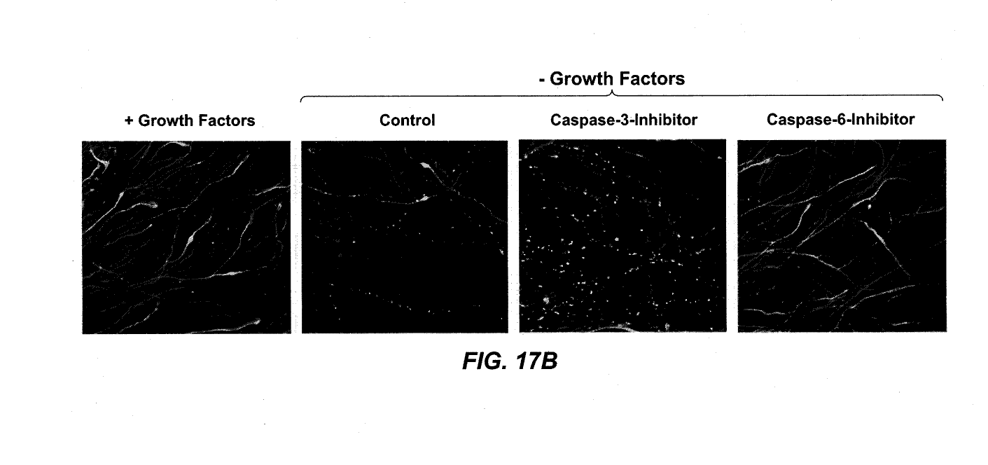

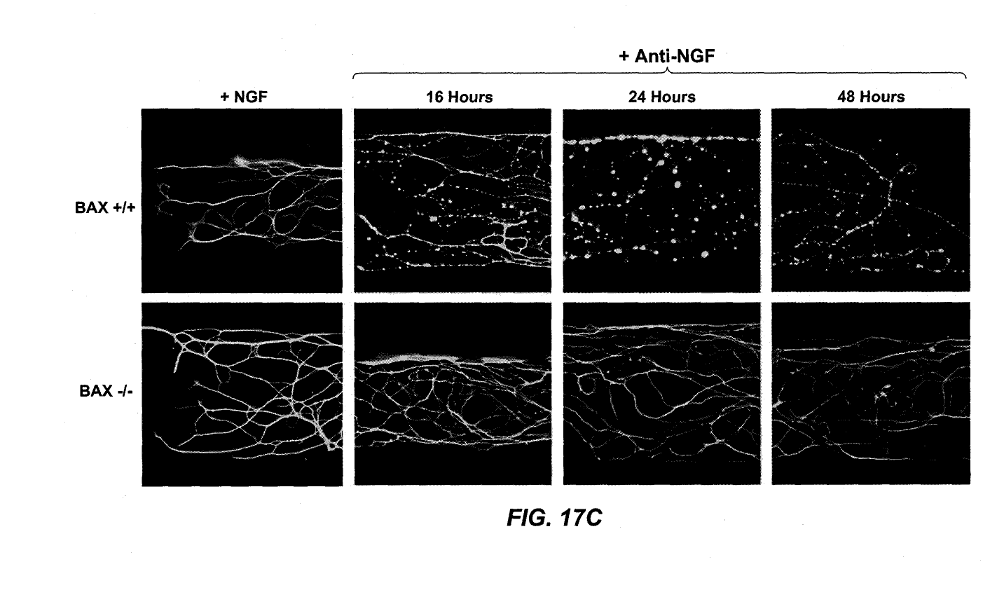

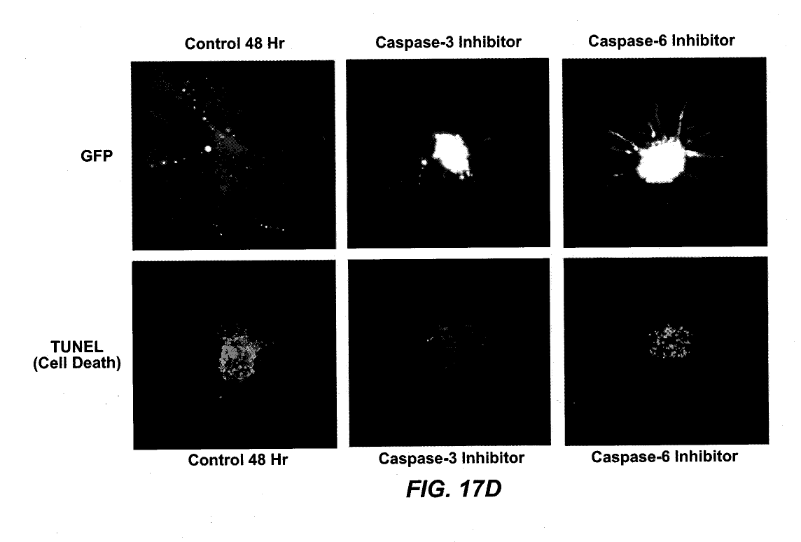

FIG. 17A shows photographs of sensory neurons cultured for 5 days and then exposed to various different culture conditions for 24 hours. As shown in FIG. 17A, axonal degeneration is delayed by inhibition of JNK and upstream caspase-8, but not by the downstream caspase-3.

In FIG. 17A, the two photographs on the left, in descending order, show sensory neurons exposed to NGF and anti-NGF antibody, respectively. In FIG. 17A, the four photographs on the right, in descending order, show sensory neurons exposed to: anti-NGF antibody and a JNK inhibitor; anti-NGF antibody and a caspase-8 inhibitor; anti-NGF antibody and a BAX inhibitor; and anti-NGF antibody and a caspase-3 inhibitor, respectively.

Materials and methods used to generate the data shown in this FIG. 17A are as follows. The NGF deprivation assay in Campenot Chambers was carried out as described above in Example 8. The small molecule JNK inhibitor, SP 600125, was used in this assay at 1 uM final concentration (SP 600125, Cat. No. 1496, Tocris Bioscience). The Caspase-3 inhibitor, Z-DEVD-FMK, was used in this assay at 10 uM (Z-DEVD-FMK, Cat. No. 264155, Calbiochem). The Caspase-8 inhibitor Z-IETD-FMK used in this assay at 10 uM (Z-IETD-FMK, Cat. No. FMK007, R&D Systems). The BAX inhibitory peptide was used at 10 uM to block neuronal cell death (Bax-V5, Tocris Inc). The Bax null mouse line (Bax-R1) was described previously (Deckwerth et al., Neuron, Vol. 17, 401-411, 1996) and was obtained from Jackson Lab Immunofluorescence labeling of sensory axons with TUJ1 antibody (1:500, Covance) was carried out as described in Examples 1, 7 and 8. To visualize immunofluorescently labeled sensory axons in axonal compartments of the Campenot Chambers, pictures were taken on the Axioplan-2 Imaging Zeiss microscope using AxioVision40 Release 4.5.0.0 SP1 (March 2006) computer software from Carl Zeiss Imaging Solutions.

FIG. 17B provides photographs of motor neurons from E12.5 motor neuron explant cultures and show that caspase-3 functions in cell bodies, while caspase-6 functions in axons.

In FIG. 17B from left to right, the four photographs show neurons cultured with: (1) growth factors; (2) without growth factors and in the absence of caspase inhibitors (a control); (3) without growth factors in the presence of a caspase-3 inhibitor; and (4) without growth factors in the presence of a caspase-6 inhibitor, respectively.

Materials and methods used to generate the data shown in this FIG. 17B are as follows. The Caspase-3 inhibitor, Z-DEVD-FMK, was used in this assay at 10 uM (Z-DEVD-FMK, Cat. No. 264155, Calbiochem). The Caspase-6 inhibitor, Z-VEID-FMK, was used in this assay at 10 uM (Z-VEID-FMK, Cat. No. 550379, Becton, Dickinson and Company PHARMINGEN Division). The motor neuron ventral spinal cord survival assay was carried out as described in Example 6 above Immunofluorescence labeling of motor axons with TUJ1 antibody (1:500, Covance) was carried out as described in Examples 1, 7 and 8. To visualize immunofluorescently labeled motor axons, pictures were taken on the Axioplan-2 Imaging Zeiss microscope using AxioVision40 Release 4.5.0.0 SP1 (March 2006) computer software from Carl Zeiss Imaging Solutions.

FIG. 17C provides photographs of sensory neurons cultured for 5 days and then exposed to various different culture conditions for 24 hours. The data in FIG. 17C shows that while Caspase-3 does not appear to be required for axon degeneration, BAX is.

In FIG. 17C from left to right, the top four photographs show BAX+/+ neurons cultured with: NGF; and then in the presence of anti-NGF antibodies (i.e. NGF deprivation) for 16, 24 and 48 hours, respectively. The bottom four photographs correspondingly show BAX−/− neurons cultured with: NGF; and then anti-NGF antibodies for 16, 24 and 48 hours, respectively.

Materials and methods used to generate the data shown in this FIG. 17C are as follows. The NGF deprivation assay in Campenot Chambers was carried out as described above in Example 8 above. The Bax null mouse line (Bax-R1) was described previously (Deckwerth et al., Neuron 17:401-411, 1996) and was obtained from Jackson Lab. The NGF antibody was used in the NGF deprivation assay in the axonal compartment of Campenot Chambers (monoclonal function-blocking anti-NGF #911, Genentech, 20 ug/ml). Immunofluorescence labeling of sensory axons with TUJ1 antibody (1:500, Covance) was carried out as described in Examples 1, 7 and 8. To visualize immunofluorescently labeled sensory axons in axonal compartments of the Campenot Chambers, pictures were taken on the Axioplan-2 Imaging Zeiss microscope (in green fluorescence channel) using AxioVision40 Release 4.5.0.0 SP1 (March 2006) computer software from Carl Zeiss Imaging Solutions.

FIG. 17D provides photographs of cultures of E13 rat explant commissural neurons cultured under different culture conditions for 24 hours. The data in FIG. 17D show that Caspase-3 functions in cell bodies, while caspase-6 functions in axons.

In FIG. 17D from left to right, the top three photographs show a GFP analysis of control neurons compared to neurons cultured with a caspase-3 or a caspase-6 inhibitor, respectively. The bottom three photographs correspondingly show a TUNEL (cell death) analysis of control neurons compared to neurons cultured with a caspase-3 or a caspase-6 inhibitor, respectively.

Materials and methods used to generate the data shown in this FIG. 17D are as follows. Commissural explant cultures and survival assay were carried out as described in Example 2. Programmed cell death in commissural cell bodies was visualized in commissural explant cultures by TUNNEL assays as described in Example 7 above. Commissural explants were fixed in 4% PFA/PBS and processed for the detection of programmed cell death (apoptosis) at single cell level, based on labeling of DNA strand breaks (TUNNEL technology) using the In situ Cell Death Detection Kit (Cat. No. 11 684 795 910, Roche) according to manufacturer's instructions manual (Roche). Apoptosis in cell bodies of commissural sensory and motor explant cultures was analyzed by fluorescence microscopy (FIG. 17D). The Caspase-3 inhibitor, Z-DEVD-FMK, was used in this assay at 10 uM (Z-DEVD-FMK, Cat. No. 264155, Calbiochem). The Caspase-6 inhibitor, Z-VEID-FMK, was used in this assay at 10 uM (Z-VEID-FMK, Cat. No. 550379, Becton, Dickinson and Company, PHARMINGEN Division). To visualize GFP-labeled commissural axons, pictures were taken on the Axiovert 200 Zeiss inverted microscope (in the green fluorescence channel for GFP) using AxioVision40 Release 4.5.0.0 SP1 (March 2006) computer software from Carl Zeiss Imaging Solutions. To visualize fluorescently labeled TUNNEL-positive apoptotic cell bodies, pictures were taken on the Axioplan-2 Imaging Zeiss microscope (in red fluorescence channel for TUNNEL) using AxioVision40 Release 4.5.0.0 SP1 (March 2006) computer software from Carl Zeiss Imaging Solutions.

### Example 14

### DR6 Antagonist Activity in Animal Models

A number of animal models associated with different neurodegenerative diseases can be employed by the skilled artisan to examine the effects of DR6 antagonists in vivo. For example, APP/RK transgenic mice express a mutant amyloid precursor protein polypeptide and exhibit severe neurodegeneration and apoptosis. APP/RK transgenic mice therefore provide a model of Alzheimer's disease which can be used to examine the effects of DR6 antagonists on the pathological processes associated with this syndrome that are observed in this animal model (see, e.g. Moechars et al., Neuroscience 91(3):819-830 (1999)). A variety of other transgenic murine lines such as the APP23 and JNPL3 transgenic lines express mutant Alzheimer's associated polypeptides and further exhibit neuronal cell loss. APP23 and JNPL3 transgenic mice thus provide alternative models of Alzheimer's disease in which DR6 antagonists may be administered (see, e.g. McGowan et al., Trends in Genetics 22(5) (2006).

G93A SOD1 transgenic mice express a human superoxide dismutase mutant polypeptide and exhibit elevated levels of caspase-3 expression as well as motor neuron apoptosis. G93A SOD1 transgenic mice provide a model of amyotrophic lateral sclerosis which can be used to examine the effects of DR6 antagonists (see, e.g. Tokuda et al., Brain Res. 1148: 234-242 (2007); and Wang et al., Eur. J. Neurosci. 26(3): 633-641 (2007)). R6/2 transgenic mice express exon-1 of huntington with an expanded N-terminal polyglutamate repeat under control of its native promoter and exhibit progressive neuropathologic changes reminiscent of Huntington's disease in humans (see, e.g. Mangarini et al. Cell, 87, 493-506 (1996); Chen et al., Nat. Med. 6, 797-801 (2000)). R6/2 transgenic mice provide a model of Huntington's disease which can be used to examine the effects of DR6 antagonists on the pathological processes associated with this syndrome that are observed in this animal model (see, e.g. Wang et al., Eur. J. Neurosci. 26: 633-641 (2007)). PK-KO transgenic mice do not express the protein product of the Park-2 gene, exhibit abnormalities that resemble Parkinson's disease, and possess neurons that are more susceptible to apoptosis than those from wild type mice (see, e.g. Casarejos et al., J. Neurochem. 97(4): 934-46 (2006)). PK-KO transgenic mice provide a model of Parkinson's disease which can be used to characterize the effects of DR6 antagonists on the pathological processes associated with this syndrome that are observed in this animal model. In addition, a number of transgenic mouse lines such as Smn−/−SMN2 mice, transgenic mice carrying pure 239 trinucleotide CAG repeats under a human AR promoter, as well as transgenic double knockouts of the native mouse Smn gene having at least one copy of human SMN^{C} gene that functions in a murine background all either do not express or express altered versions of the protein product of the survival motor neuron genes and consequently exhibit abnormalities that resemble Spinal Muscular Atrophy disease (see, e.g. Hsiu et al., Nature Genetics 24, 66-70 (2000); Ferri et al., Neuroreport 15(2): 275-280 (2004); Ferri et al., Curr. Biol. 2003 Apr. 15; 13(8):669-73; and Rossol et al., J. Cell Biol., Volume 163(4):801-812 (2003)). Such transgenic murine lines consequently provide models of Spinal Muscular Atrophy which can be used to characterize the effects of DR6 antagonists on the pathological processes associated with this syndrome that are observed in this animal model.

Animal models of neurological conditions or disorders including those noted above can be used to examine the effects of the DR6 antagonists disclosed herein, for example one or more antibodies that binds DR6 (e.g. the 3F4.4.8, 4B6.9.7, or 1E5.5.7 monoclonal antibody), and/or one or more soluble forms of DR6 that bind APP (e.g. one that comprises amino acids 1-354 of SEQ ID NO: 1), and/or one or more antibodies that bind APP (e.g. the 22C11 monoclonal antibody) as well as these agents in combination with each other and/or other therapeutic agents known in the art.

In illustrative protocols for the experimental testing of one or more of the DR6 antagonists disclosed herein, a number of age and gender matched animals from an animal model (e.g. 6 month old female APP/RK transgenic mice) can be assigned to one of multiple test and/or control groups. A first test group of these animals can then be administered a selected DR6 antagonist according to a specific administration protocol (for example an intraperitoneal injection of an DR6 antagonist antibody at 20 mg/kg body weight for each injection every two weeks for a period of six months). Conditions for other test groups can be varied according to standard practices, for example: by administering a different dose of the DR6 antagonist (e.g. 1, 5, 10, 15 mg/kg body weight); by administering a different schedule of the DR6 antagonist (e.g. an injection every week for a period of 12 months); by administering a different DR6 antagonist (e.g. a DR6 immunoadhesin); by using a combination of agents (e.g. the DR6 antagonist in combination with a cholinesterase inhibitor); by using a different route of administration (e.g. intravenous administration) etc. One or more groups of animals can serve as a control, for example one that receives sterile phosphate buffered saline according to the same course of administration as a test group that receives the DR6 antagonist.

At some period of time after receiving the DR6 antagonist, a test and a matched control group of these animals can then be compared for example to examine and/or characterize the effects of DR6 antagonists in vivo. For example, samples comprising neuronal cells from a specific tissue or organ (e.g. the brain) from test and control groups of these animals can be evaluated by a technique such as magnetic resonance microscopy and/or immunohistochemical analysis in order to compare the status of neuronal cells in these groups (see, e.g. Petrik et al., Neuromolecular Med. 9(3):216-29 (2007)). Alternatively, samples obtained from these groups can be evaluated by a technique such as multi-photon microscopy in order to demonstrate phenomena such as altered neurite trajectory, dendritic spine loss or thinning of dendrites (see, e.g. Tsai et al., Nat. Neurosci. 7:1181-1183 (2004): and Spires et al., J. Neurosci. 25:7278-7287 (2005)). Alternatively, blood or other tissue samples obtained from these groups can be subjected to ELISA protocols designed to measure levels of markers of inflammation and/or apoptosis such as IL-1β, TNF-α, IL-10, p53 protein, interferon-γ, or NF-kappaB (see, e.g. Rakover et al., Neurodegener. Dis. 4(5):392-402 (2007); and Mogi et al., Neurosci Lett. 414(1):94-7 (2007)). Alternatively, animals from a test and a matched control group can be compared in behavioral test paradigms known in the art, for example the Morris water maze or object recognition tests (see, e.g., Hsiao et al., Science 274, 99-102 (1996); Janus et al., Nature 408:979-982 (2000); Morgan et al., Nature 408:982-985 (2000); and Ennaceur et al., Behav. Brain Res. 1988, 31:47-59). The results of comparisons between test and matched control groups of animals will allow those skilled in the art to examine the effects of DR6 antagonists in vivo in the animal models.

Examples 1-13, the data included therein and the associated characterization of this data evidences that DR6 antagonists will for example, inhibit the apoptosis of neuronal cells in vivo. In particular, Examples 1-13 above teach for example that: (1) DR6 induces apoptosis in a wide variety of neuronal cells; (2) APP is a cognate ligand for DR6 which binds DR6 and triggers DR6 mediated apoptosis; and (3) DR6 antagonists which inhibit the DR6/APP binding interaction in vitro consequently inhibit DR6 mediated apoptosis in vitro. In view of Applicants' findings and disclosure, one of skill in this art will reasonably expect DR6 antagonists to inhibit DR6 mediated apoptosis in vivo. For this reason, the skilled artisan will reasonably expect animal models such as those noted above and the associated techniques for examining the various pathological processes observed these animal models to confirm the biological activity of DR6 antagonists, as described herein.

### Example 15

### Ra.1 (“1E5.5.7”), Ra.2, Ra.3 (“3F4.4.8”) and Ra.4 Antibody Treatment in an Animal Model of Spinal Muscular Atrophy

Spinal muscular atrophy (SMA) is a recessive motor neuron disease that affects motor neurons in the anterior horn of the spinal cord, and is believed to result from the reduction of SMN (survival motor neuron) protein. An animal model of SMA is the transgenic mouse line having the strain designation Strain Designation: FVB.Cg-Tg(SMN2*delta7)4299Ahmb Tg(SMN2)89Ahmb Smn1tm1Msd/J (JAX 5025), (see, e.g. Le et al., Human Molecular Genetics 14(6):845-857 (2005). This triple mutant mouse harbors two transgenic alleles and a single targeted mutant. The Tg(SMN2*delta7)4299Ahmb allele consists of a SMA cDNA lacking exon 7 whereas the Tg(SMN2)₈₉Ahmb allele consists of the entire human SMN2 gene. In the description below, this strain is also referred to as the Delta 7 SMA KO Model.

Mice that are homozygous for the targeted mutant Smn allele and homozygous for the two transgenic alleles exhibit symptoms and neuropathology similar to patients afflicted with proximal spinal muscular atrophy (SMA). At birth, triple mutants are noticeably smaller than normal littermates. By day 5, signs of muscle weakness are apparent and become progressively more pronounced over the following week as the mice display an abnormal gait, shakiness in the hind limbs and a tendency to fall over. Mean survival is approximately 13 days. Triple mutant mice further exhibit impaired responses to surface righting, negative geotaxis and cliff aversion but not to tactile stimulation. Spontaneous motor activity and grip strength are also significantly impaired in these mice (see, e.g. Butchbach et al., Neurobiol Dis. 27(2):207-19 (2007)). The following protocols are designed to determine the effect of certain antibodies, such as DR6 antagonist antibodies, and doses on the survival, body weight and muscle tone of Delta 7 SMA Model mice (KO).

As noted above, mice used in this study can be Delta-7 SMA (JAX 5025) KO Model (smn−/−; SMN2+/+; d7+/+). At birth, litters can be randomly culled to 10 animals (or some other number) with, for example, equal numbers of males and females removed. Following this protocol, litters can be culled to 8 mice by time of first dosing (P3). Any litter with less than 6 pups can be voided from the study. Mice can be tail snipped at birth (P0) from litters born between Monday and Wednesday. Genotyping can be performed by a variety of methodologies known in the art, for example using automated genotyping service screens for transgenic, knock-out, and knock-in mutations in biopsies that are commercially available from molecular diagnostics companies such as Transnetyx Inc. Such genotype data is typically available within 48 hours after birth.

Mice born for example on Monday-Wednesday can be used in illustrative experiments. Mice can be dosed IP starting at P3. A typical number in the study can be: (1) for example on average, 10 KOs (5 males and 5 females) controls with vehicle such as sterile PBS; (2) for example on average 10 KOs (5 males and 5 females) with a first dose of the respective antibody that comprises 20 mg/kg; and (3) for example on average 10 KOs (5 males and 5 females) with second dose of the respective DR6 antibody that comprises 5 mg/kg. Each animal can receive an IP dose of the respective 4B6.9.7, IE5.5.7, 3F4.4.8, and 2C7.3.7 antibody twice weekly. The 2C7.3.7 antibody (Genentech, Inc.) is an antibody which binds to DR6, but is not function-blocking. The 3B11.7.7 antibody (Genentech, Inc.) is an antibody which binds to DR6, but may enhance or stimulate DR6 activity.

The 4B6.9.7, IE5.5.7, 3F4.4.8 and 2C7.3.7 antibodies can be stored at 4° C. These antibodies can be warmed to room temperature prior to dosing if necessary. Typical vehicles such as PBS can be used. While the 4B6.9.7, IE5.5.7, 3F4.4.8, and 2C7.3.7 monoclonal antibodies in this Example were generated using a human DR6 polypeptide sequence as an immunogen, all of these antibodies react with both human as well as rat and mouse DR6 as shown by protocols such as the axon degeneration and apoptosis assays described in Example 7.

In one illustrative embodiment, the DR6 antagonists evaluated can be the antagonist antibodies: 4B6.9.7, IE5.5.7, 3F4.4.8 and 2C7.3.7; the number of treatment groups per antibody can be 2 (with 10 animals per group); the route of administration can be IP; and the dose range can be 5 and 20 mg/kg. Optionally the groups can be as follows: (1) 4B6.9.7: 5 mg/kg IP; (2) 4B6.9.7: 20 mg/kg IP; (3) IE5.5.7: 5 mg/kg IP; (4) IE5.5.7: 20 mg/kg IP; (5) 3F4.4.8: 5 mg/kg IP; (6) 3F4.4.8: 20 mg/kg IP; (7) 2C7.3.7: 5 mg/kg IP; (8) 2C7.3.7: 20 mg/kg IP; and (9) Vehicle (PBS) IP. In this protocol, mice can be weighed daily. At Postnatal Day (PND) 10, 12 and 14, body weight of each pup in the litter can be taken. At PND 6, 8, 10, 12, 14 and 16, muscle tone assessment can be performed on each animal in the study. (see, e.g. the illustrative Phenotyping protocol provided below).

At day of birth (P0) pups can be tattooed using non-toxic ink applied under the skin and a tail snip sample is taken for genotyping (the results can be normally available within 48 hrs). On the day of the experiment (P3) the dams with neonates can be brought to the experimental room at the same time everyday and left undisturbed for at least 10 min before testing begins. The pups can be first tested in the geotaxis test and then in the tube test (2 consecutive trials on the tube test). A pup can be placed on a heated pad until all the pups in the litter are tested and then all the pups can be returned to their dam (the pups can be mixed with their cage bedding to minimize rejection by the dam following handling). The survival and body weight can be checked every day from birth until weaning. The effect of the drug on the neonate axial body temperature is normally assessed during the chronic MTD study performed previously. Body temperature: one reading of the axial body temperature can be taken at the specified age.

Mice in the test and control groups can be examined for differences by examination protocols including Geotaxis. Geotaxis tests the ability of the animal to orient itself when placed face down on an inclined platform. This test measures motor coordination and the vestibular system.

Survival evaluation can be performed using Kaplan-Meier analysis with Mantel-Cox as the post-hoc test.

To analyze data with repeated measurements over time, Mixed Effects Models (also known as Mixed ANOVA models) can be employed. This approach is based on likelihood estimation rather than moment estimation as in typical repeated-measures ANOVA analysis, but it is more robust to missing values due to mice fatalities over time. All models can be fit using the PROC MIXED procedure in SAS 9.1.3. (SAS Institute, Cary, N.C.). Treatment is the most important factor in the model. Gender and Day can be also considered, as well as their interaction with treatment.

Study endpoints can be death.

Animals can be further evaluated by a methodology such as those noted in Example 14, e.g. histological analysis. In addition, Serum/blood can be evaluated to determine

4B6.9.7, IE5.5.7, 3F4.4.8 and 2C7.3.7 serum concentrations.

### Deposit of Material

The following materials have been deposited with the American Type Culture Collection, 10801 University Blvd., Manassas, Va. 20110-2209, USA (ATCC):

| Material | ATCC Deposit No. | Deposit Date |
|---|---|---|
| 3F4.4.8 | PTA-8095 | Dec. 21, 2006 |
| 4B6.9.7 | PTA-8094 | Dec. 21, 2006 |
| 1E5.5.7 | PTA-8096 | Dec. 21, 2006 |

This deposit was made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit. The deposit will be made available by ATCC under the terms of the Budapest Treaty, and subject to an agreement between Genentech, Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. §122 and the Commissioner's rules pursuant thereto (including 37 CFR §1.14 with particular reference to 886 OG 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

The foregoing written description is considered to be sufficient to enable one skilled in the art to practice the invention. The present invention is not to be limited in scope by the examples presented herein. Indeed, various modifications of the invention in addition to those shown and described herein will become apparent to those skilled in the art from the foregoing description and fall within the scope of the appended claims.

What is claimed is:

1. A method for screening for compounds that inhibit neurodegeneration comprising:
(a) culturing neurons expressing APP on their surface; and
(b) stimulating APP shedding in said neurons in the presence or absence of a candidate compound;
wherein a reduction of observed shedding of APP in the presence of said candidate compound as compared to observed shedding of APP in the absence of said candidate compound indicates that said candidate compound is an inhibitor of neurodegeneration.

2. The method of claim 1 wherein shedding is stimulated by trophic factor deprivation.

3. The method of claim 2 wherein said trophic factor is NGF.

4. The method of claim 1 wherein said neurons are sensory neurons.

5. The method of claim 1 wherein said neurons are motor neurons.

6. The method of claim 1 wherein said shedding is stimulated by mechanical damage of said neuron.

7. The method of claim 1 wherein said candidate compound reduces observed shedding compared to shedding observed in the absence of said candidate compound by 10-30%.

8. The method of claim 1 wherein said candidate compound reduces observed shedding compared to shedding observed in the absence of said candidate compound by 30-50%.

9. The method of claim 1 wherein said candidate compound reduces observed shedding compared to shedding observed in the absence of said candidate compound by 50-70%.

10. The method of claim 1 wherein said candidate compound reduces observed shedding compared to shedding observed in the absence of said candidate compound by 70-90%.

11. The method of claim 1 wherein said candidate compound reduces observed shedding compared to shedding observed in the absence of said candidate compound by 90-100%.

12. A method for screening for compounds that inhibit neurodegeneration comprising:
(a) culturing neurons expressing APP on their surface; and
(b) stimulating neurodegeneration of said neurons in the presence or absence of a candidate compound;
wherein a reduction of observed neurodegeneration in the presence of said candidate compound as compared to observed neurodegeneration in the absence of said candidate compound indicates that said candidate compound is an inhibitor of neurodegeneration.

13. The method of claim 12 wherein neurodegeneration is stimulated by trophic factor deprivation.

14. The method of claim 13 wherein said trophic factor is NGF.

15. The method of claim 12 wherein said neurons are sensory neurons.

16. The method of claim 12 wherein said neurons are motor neurons.

17. The method of claim 12 wherein said shedding is stimulated by mechanical damage of said neuron.

18. The method of claim 12 wherein said candidate compound reduces observed neurodegeneration compared to neurodegeneration observed in the absence of said candidate compound by 10-30%.

19. The method of claim 12 wherein said candidate compound reduces observed neurodegeneration compared to neurodegeneration observed in the absence of said candidate compound by 30-50%.

20. The method of claim 12 wherein said candidate compound reduces observed neurodegeneration compared to neurodegeneration observed in the absence of said candidate compound by 50-70%.

21. The method of claim 12 wherein said candidate compound reduces observed neurodegeneration compared to neurodegeneration observed in the absence of said candidate compound by 70-90%.

22. The method of claim 12 wherein said candidate compound reduces observed neurodegeneration compared to neurodegeneration observed in the absence of said candidate compound by 90-100%.


SEQUENCE LISTING (15 sequences)

SEQ ID NO: 1 - protein, 655 aa, Homo sapiens
MGTSPSSSTALASCSRIARRATATMIAGSLLLLGFLSTTTAQPEQKASNLIGTYRHVDRATGQVLTCDKCPAGTYVSEHCTNTSLRVCSSCPVGTFTRHENGIEKCHDCSQPCPWPMIEKLPCAALTDRECTCPPGMFQSNATCAPHTVCPVGWGVRKKGTETEDVRCKQCARGTFSDVPSSVMKCKAYTDCLSQNLVVIKPGTKETDNVCGTLPSFSSSTSPSPGTAIFPRPEHMETHEVPSSTYVPKGMNSTESNSSASVRPKVLSSIQEGTVPDNTSSARGKEDVNKTLPNLQVVNHQQGPHHRHILKLLPSMEATGGEKSSTPIKGPKRGHPRQNLHKHFDINEHLPWMIVLFLLLVLVVIVVCSIRKSSRTLKKGPRQDPSAIVEKAGLKKSMTPTQNREKWIYYCNGHGIDILKLVAAQVGSQWKDIYQFLCNASEREVAAFSNGYTADHERAYAALQHWTIRGPEASLAQLISALRQHRRNDVVEKIRGLMEDTTQLETDKLALPMSPSPLSPSPIPSPNAKLENSALLTVEPSPQDKNKGFFVDESEPLLRCDSTSSGSSALSRNGSFITKEKKDTVLRQVRLDPCDLQPIFDDMLHFLNPEELRVIEEIPQAEDKLDRLFEIIGVKSQEASQTLLDSVYSHLPDLL

SEQ ID NO: 2 - DNA, 3662 bp, Homo sapiens
gccaccacgtgtgtccctgcgcccggtggccaccgactcagtccctcgccgaccagtctgggcagcggaggagggtggttggcagtggctggaagcttcgctatgggaagttgttcctttgctctctcgcgcccagtcctcctccctggttctcctcagccgctgtcggaggagagcacccggagacgcgggctgcagtcgcggcggcttctccccgcctgggcggccgcgccgctgggcaggtgctgagcgcccctagagcctcccttgccgcctccctcctctgcccggccgcagcagtgcacatggggtgttggaggtagatgggctcccggcccgggaggcggcggtggatgcggcgctgggcagaagcagccgccgattccagctgccccgcgcgccccgggcgcccctgcgagtccccggttcagccatggggacctctccgagcagcagcaccgccctcgcctcctgcagccgcatcgcccgccgagccacagccacgatgatcgcgggctcccttctcctgcttggattccttagcaccaccacagctcagccagaacagaaggcctcgaatctcattggcacataccgccatgttgaccgtgccaccggccaggtgctaacctgtgacaagtgtccagcaggaacctatgtctctgagcattgtaccaacacaagcctgcgcgtctgcagcagttgccctgtggggacctttaccaggcatgagaatggcatagagaaatgccatgactgtagtcagccatgcccatggccaatgattgagaaattaccttgtgctgccttgactgaccgagaatgcacttgcccacctggcatgttccagtctaacgctacctgtgccccccatacggtgtgtcctgtgggttggggtgtgcggaagaaagggacagagactgaggatgtgcggtgtaagcagtgtgctcggggtaccttctcagatgtgccttctagtgtgatgaaatgcaaagcatacacagactgtctgagtcagaacctggtggtgatcaagccggggaccaaggagacagacaacgtctgtggcacactcccgtccttctccagctccacctcaccttcccctggcacagccatctttccacgccctgagcacatggaaacccatgaagtcccttcctccacttatgttcccaaaggcatgaactcaacagaatccaactcttctgcctctgttagaccaaaggtactgagtagcatccaggaagggacagtccctgacaacacaagctcagcaagggggaaggaagacgtgaacaagaccctcccaaaccttcaggtagtcaaccaccagcaaggcccccaccacagacacatcctgaagctgctgccgtccatggaggccactgggggcgagaagtccagcacgcccatcaagggccccaagaggggacatcctagacagaacctacacaagcattttgacatcaatgagcatttgccctggatgattgtgcttttcctgctgctggtgcttgtggtgattgtggtgtgcagtatccggaaaagctcgaggactctgaaaaaggggccccggcaggatcccagtgccattgtggaaaaggcagggctgaagaaatccatgactccaacccagaaccgggagaaatggatctactactgcaatggccatggtatcgatatcctgaagcttgtagcagcccaagtgggaagccagtggaaagatatctatcagtttctttgcaatgccagtgagagggaggttgctgctttctccaatgggtacacagccgaccacgagcgggcctacgcagctctgcagcactggaccatccggggccccgaggccagcctcgcccagctaattagcgccctgcgccagcaccggagaaacgatgttgtggagaagattcgtgggctgatggaagacaccacccagctggaaactgacaaactagctctcccgatgagccccagcccgcttagcccgagccccatccccagccccaacgcgaaacttgagaattccgctctcctgacggtggagccttccccacaggacaagaacaagggcttcttcgtggatgagtcggagccccttctccgctgtgactctacatccagcggctcctccgcgctgagcaggaacggttcctttattaccaaagaaaagaaggacacagtgttgcggcaggtacgcctggacccctgtgacttgcagcctatctttgatgacatgctccactttctaaatcctgaggagctgcgggtgattgaagagattccccaggctgaggacaaactagaccggctattcgaaattattggagtcaagagccaggaagccagccagaccctcctggactctgtttatagccatcttcctgacctgctgtagaacatagggatactgcattctggaaattactcaatttagtggcagggtggttttttaattttcttctgtttctgatttttgttgtttggggtgtgtgtgtgtgtttgtgtgtgtgtgtgtgtgtgtgtgtgtgtgtgtgtttaacagagaatatggccagtgcttgagttctttctccttctctctctctcttttttttttaaataactcttctgggaagttggtttataagcctttgccaggtgtaactgttgtgaaatacccaccactaaagttttttaagttccatattttctccattttgccttcttatgtattttcaagattattctgtgcactttaaatttacttaacttaccataaatgcagtgtgacttttcccacacactggattgtgaggctcttaacttcttaaaagtataatggcatcttgtgaatcctataagcagtctttatgtctcttaacattcacacctactttttaaaaacaaatattattactatttttattattgtttgtcctttataaattttcttaaagattaagaaaatttaagaccccattgagttactgtaatgcaattcaactttgagttatcttttaaatatgtcttgtatagttcatattcatggctgaaacttgaccacactattgctgattgtatggttttcacctggacaccgtgtagaatgcttgattacttgtactcttcttatgctaatatgctctgggctggagaaatgaaatcctcaagccatcaggatttgctatttaagtggcttgacaactgggccaccaaagaacttgaacttcaccttttaggatttgagctgttctggaacacattgctgcactttggaaagtcaaaatcaagtgccagtggcgccctttccatagagaatttgcccagctttgctttaaaagatgtcttgttttttatatacacataatcaataggtccaatctgctctcaaggccttggtcctggtgggattccttcaccaattactttaattaaaaatggctgcaactgtaagaacccttgtctgatatatttgcaactatgctcccatttacaaatgtaccttctaatgctcagttgccaggttccaatgcaaaggtggcgtggactccctttgtgtgggtggggtttgtgggtagtggtgaaggaccgatatcagaaaaatgccttcaagtgtactaatttattaataaacattaggtgtttgttaaaaaaaaaaaaaaaaaaaaaaaaaaaaa

SEQ ID NO: 3 - DNA, 300 bp, Mus musculus
gagcagaaacggctcctttattaccaaagaaaagaaggacacagtgttgcggcaggtccgcctggacccctgtgacttgcagcccatctttgatgacatgctgcatatcctgaaccccgaggagctgcgggtgattgaagagattccccaggctgaggacaaactggaccgcctcttcgagatcattggggtcaagagccaagaagccagccagaccctcttggactctgtgtacagtcatcttcctgacctattgtagaacacaggggcactgcattctgggaatcaacctactggcgg

SEQ ID NO: 4 - protein, 582 aa, Artificial sequence
  sequence is synthesized
MGTSPSSSTALASCSRIARRATATMIAGSLLLLGFLSTTTAQPEQKASNLIGTYRHVDRATGQVLTCDKCPAGTYVSEHCTNTSLRVCSSCPVGTFTRHENGIEKCHDCSQPCPWPMIEKLPCAALTDRECTCPPGMFQSNATCAPHTVCPVGWGVRKKGTETEDVRCKQCARGTFSDVPSSVMKCKAYTDCLSQNLVVIKPGTKETDNVCGTLPSFSSSTSPSPGTAIFPRPEHMETHEVPSSTYVPKGMNSTESNSSASVRPKVLSSIQEGTVPDNTSSARGKEDVNKTLPNLQVVNHQQGPHHRHILKLLPSMEATGGEKSSTPIKGPKRGHPRQNLHKHFDINEHLPWMIPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 2088 bp, Homo sapiens
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 6 - protein, 695 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 7 - protein, 751 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAIPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 8 - DNA, 2310 bp, Homo sapiens
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaac

SEQ ID NO: 9 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 10 - protein, 210 aa, Artificial sequence
  sequence is synthesized
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDHHHHHH

SEQ ID NO: 11 - DNA, 21 bp, Artificial sequence
  sequence is synthesized
aaucuguugaguucaugccuu

SEQ ID NO: 12 - DNA, 21 bp, Artificial sequence
  sequence is synthesized
caauaggucaggaagauggcu

SEQ ID NO: 13 - DNA, 37 bp, Artificial sequence
  sequence is synthesized
ggactctgtgtacagtcacctcccagatctgttatag

SEQ ID NO: 14 - protein, 349 aa, Mus musculus
MGTRASSITALASCSRTAGQVGATMVAGSLLLLGFLSTITAQPEQKTLSLPGTYRHVDRTTGQVLTCDKCPAGTYVSEHCTNMSLRVCSSCPAGTFTRHENGIERCHDCSQPCPWPMIERLPCAALTDRECICPPGMYQSNGTCAPHTVCPVGWGVRKKGTENEDVRCKQCARGTFSDVPSSVMKCKAHTDCLGQNLEVVKPGTKETDNVCGMRLFFSSTNPPSSGTVTFSHPEHMESHDVPSSTYEPQGMNSTDSNSTASVRTKVPSGIEEGTVPDNTSSTSGKEGTNRTLPNPPQVTHQQAPHHRHILKLLPSSMEATGEKSSTAIKAPKRGHPRQNAHKHFDINEH

SEQ ID NO: 15 - protein, 354 aa, Artificial sequence
  sequence is synthesized
MGTRASSITALASCSRTAGQVGATMVAGSLLLLGFLSTITAQPEQKTLSLPGTYRHVDRTTGQVLTCDKCPAGTYVSEHCTNMSLRVCSSCPAGTFTRHENGIERCHDCSQPCPWPMIERLPCAALTDRECICPPGMYQSNGTCAPHTVCPVGWGVRKKGTENEDVRCKQCARGTFSDVPSSVMKCKAHTDCLGQNLEVVKPGTKETDNVCGMRLFFSSTNPPSSGTVTFSHPEHMESHDVPSSTYEPQGMNSTDSNSTASVRTKVPSGIEEGTVPDNTSSTSGKEGTNRTLPNPPQVTHQQAPHHRHILKLLPSSMEATGEKSSTAIKAPKRGHPRQNAHKHFDINEHHHHHH